(19) United States
(12) Patent Application Publication
(10) Pub. No.: US 2026/0102635 A1
(43) Pub. Date: Apr. 16, 2026

(54) DEVICE FOR ULTRA-WIDEBAND MICROMECHANICAL THERAPY AND METHOD OF ITS OPERATION

(71) Applicant: EVOLUS TECHNOLOGIES LTD., Haifa (IL)

(72) Inventors: Aleksandr MARCHENKO, Haifa (IL); Audrius MICIUKEVICIUS, Santa Cruz de Tenerife (ES)

(21) Appl. No.: 19/366,551

(22) Filed: Oct. 23, 2025

Related U.S. Application Data
(63) Continuation-in-part of application No. 18/220,626, filed on Jul. 11, 2023, now abandoned.
(60) Provisional application No. 63/359,907, filed on Jul. 11, 2022.
(60) Provisional application No. 63/459,661, filed on Apr. 16, 2023.

(30) Foreign Application Priority Data
Jul. 12, 2022 (IL) 294690

(51) Int. Cl. A61N 7/00

(52) U.S. Cl. CPC A61N 7/00; A61N 2007/0017; A61N 2007/0073

(57) ABSTRACT

A therapeutic device for applying impact ultra-wideband ultrasound bursts to a target location inside a biological object. The therapeutic device comprises: a generating module comprising at least one ultrasound generator configured for generating at least one ultra-wideband electrical signal of ultrasound frequency, at least one ultra-wideband ultrasound transducer connected to the at least one ultrasound generator and attachable to the biological object in proximity to an object area to be treated, and a control unit configured for controlling the at least one ultrasound generator such that said at least one electric ultra-wideband ultrasound electrical signal of ultrasound frequency is generated. The at least one ultra-wideband ultrasound transducer configured for converting said at least one electric ultra-wideband frequency signal into ultra-wideband ultrasound impact burst and transmitting thereof into said biological object target location.

### RELATED APPLICATIONS

This application is a Continuation-in-part of U.S. application Ser. No. 18/220,626, filed on Jul. 11, 2023.

This application claims the benefit of priority of Israel Patent Application No. 294690 filed on Jul. 12, 2022.

This application also claims the benefit of priority under 35 USC § 119 (e) of U.S. Provisional Patent Application Nos. 63/359,907 filed on Jul. 11, 2022, and 63/459,661 filed on Apr. 16, 2023.

The contents of the above applications are all incorporated by reference as if fully set forth herein in their entirety in their entirety.

### FIELD AND BACKGROUND OF THE INVENTION

The present invention, in some embodiments thereof, relates to a device and method for medical treatment using ultrasound waves and, more particularly, but not exclusively, to promoting tissue regeneration using pulsed ultrasound at relatively low intensities.

Low intensity pulsed ultrasound (LIPUS) is known for therapy. An example is Published U.S. Patent Application 2011/0178441 to Tyler. This application describes methods and devices for modulating the-activities of living cells. Methods comprise use of the application of ultrasound, such as low intensity, low frequency ultrasound to living cells to affect the cells and modulate the cells' activities. The ultrasound waveforms shown in this publication generally consist of pulses 10 to 100 cycles long at frequencies of about 0.5 MHz, so their bandwidths are narrowband.

Published U.S. Patent Application 2007/0249938 to Shields describes systems, devices, and methods for delivering ultrasonic treatment to a subject. An ultrasound therapy device includes waveform generator, one or more transducers, one or more sensors, and a controller. In some embodiments, the waveform generator is configured to generate a first driving signal having at least a first waveform segment and a second waveform segment different from the first waveform segments separated from each other. FIG. 3B of Shields is an exemplary multivariant narrowband waveform.

Zhouyang Shen and Philipp Niethammer, “A cellular sense of space and pressure,” Science 370, 295-296 (16 Oct. 2020), summarizes two other papers in the same issue of Science, Lomakin et al, “The nucleus acts as a ruler tailoring cell responses to spatial constraints,” Science 370, 310 (2020) and Venturini et al, “The nucleus measures shape changes for cellular proprioception to control dynamic cell behavior,” Science 370, 311 (2020). They find that embryonic, immune, and cancer cells sense confinement through deformation of their nucleus. Stretch in the nuclear membrane activates the enzyme cytosolic phospholipase A2 (cPLA2), which initiates cell blebbing and movements that may help cells to crawl within or out of narrow spaces. Both studies add to the emerging idea that the nucleus, besides its genetic functions, directly senses the cell's physical environment.

Chastin Newman, Allan Lawrie, Axel Brisken, and David Cumberland, “Ultrasound Gene Therapy: On the Road from Concept to Reality,” Echocardiography 18, 339-47 (2001), 10.1046/j.1540-8175.2001.00339.x, states that the promise of gene therapy lies in the potential to ameliorate or cure conditions that are resistant to conventional therapeutic approaches. Progress in vascular and all other fields of gene therapy has been hampered by concerns over the safety and practicality of recombinant viral vectors and the inefficiency of current nonviral transfection techniques. This review summarizes the increasing evidence that exposure of eukaryotic cells to relatively modest intensity ultrasound, within the range emitted by diagnostic transducers, either alone or in combination with other nonviral techniques, can enhance transgene expression by up to several orders of magnitude over naked DNA alone. In combination with the flexibility and excellent clinical safety profile of therapeutic and diagnostic ultrasound, these data suggest that ultrasound-assisted gene delivery has great promise as a novel approach to improve the efficiency of many forms of nonviral gene delivery.

U.S. Pat. No. 6,231,528 to Kaufman et al describes non-invasive therapeutic treatment of bone in vivo using ultrasound in conjuction with application of a biochemical compound or bone growth factor, performed by subjecting bone to an ultrasound signal supplied to an ultrasound transducer placed on the skin of a bony member, and involving a repetitive finite duration signal consisting of plural narrowband frequencies that are in the ultrasonic range to 20 MHz. Concurrent with application of the ultrasound is the utilization of a bone growth factor applied to the skin of a bony member before stimulation with ultrasound. Ultrasonic stimulation is operative to transport the bone growth factor to the bone and then to synergistically enhance the interaction of the bone growth factor with the bone, whereby to induce healing, growth and ingrowth responses.

U.S. Pat. No. 4,530,360 to Duarte describes an apparatus and method for healing bone fractures, pseudoarthroses and the like with the use of ultrasound. An ultrasound transducer, in contact with the skin of the patient, transmits narrowband ultrasound narrowband pulses to the site of the bone defect. The nominal frequency of the ultrasound is 1.5 MHz, the width of each pulse varies between 10 and 2000 microseconds, and the pulse repetition rate varies between 100 and 1000 Hz. The power level of the ultrasound is about 100 milliwatts per square centimeter. Treatments which last no more than 20 minutes per day have been found to heal defects in a wide variety of cases in less than 2 months.

It is known to use stem cell therapy to promote tissue regeneration.

### SUMMARY OF THE INVENTION

It is hence one object of the invention to disclose a therapeutic device for applying impact ultra-wideband ultrasound bursts to a target location inside a biological object. The aforesaid device comprises:
a generating module comprising at least one ultrasound generator configured for generating at least one ultra-wideband electric signal of ultrasound frequency;
at least one ultra-wideband ultrasound transducer connected to the at least one ultrasound generator and attachable to the biological object in proximity to an object area to be treated; the at least one ultra-wideband ultrasound transducer configured for converting the at least one ultra-wideband signal of ultrasound frequency into ultra-wideband ultrasound impact burst and transmitting thereof into the biological object target location;
a control unit configured for controlling the at least one ultra-wideband electric signal of ultrasound frequency is generated;
   wherein the at least one transmitted ultra-wideband ultrasound impact burst comprises a low-frequency range having a duration ranging between 0.5 and 1 us at a frequency ranging between 1 MHz and 3 MHz and a high-frequency range having a duration ranging between 10 and 50 ns at a frequency higher than 3 MHz within a frequency range of a frequency factor greater than 3; transmitting the high-frequency and low-frequency ranges is synchronized such that a period of transmitting the high-frequency range is within a period of transmitting the low-frequency range to the biological object target location.

Another object of the invention is to provide the at least one ultra-wideband ultrasound impact burst in the high-frequency range transmitted in a middle of the duration of transmitting the at least one ultra-wideband ultrasound burst in the low-frequency range to the biological object target location.

A further object of the invention is to provide the at least one ultrasound generator configured for generating a train of the ultra-wideband ultrasound impact bursts.

A further object of the invention is to provide the generating module comprising at least one ultrasound generator configured for consecutively generating a plurality of predetermined signals and a buffer configured for accumulating consecutively generated signals and in-phase transmitting the predetermined signals generated by the at least one generator and accumulated in the buffer to the at least one ultrasound transducer.

A further object of the invention is to provide the generating module comprising at least two ultrasound generator configured for generating the at least two ultra-wideband ultrasound signals at frequencies within the low-frequency and high-frequency ranges and supplementing each other and in a corrector configured for band-pass filtering the at least two ultra-wideband ultrasound signals in the low-frequency and high-frequency ranges, amplitude normalizing the at least two ultra-wideband ultrasound signals and of ultrasound frequency in-phase summing the at least two ultra-wideband ultrasound electric signals such that a summed ultra-wideband ultrasound signal when transmitted to the at least one ultrasound transducer causes generating the at least one ultra-wideband ultrasound impact burst.

A further object of the invention is to provide the therapeutic comprising a laser module configured for illuminating biological object area to be treated. The laser module comprises a laser infrared light source configured for generating a laser beam and a light modulator configured for modulating intensity of the laser beam at to generate ultrasound of greater than 20 MHz.

A further object of the invention is to provide the laser module comprising an optical element configured for focusing a modulated laser beam on a target location of the object.

A method of applying ultra-wideband ultrasound impact bursts to a biological object area. The aforesaid method comprises steps
providing a therapeutic device;
connecting at least one ultrasound transducer to the at least one ultrasound generator;
attaching the at least one ultrasound transducer to the biological object in proximity to a biological object area to be treated;
generating at least one the ultra-wideband dual-band electric signal of ultrasound frequency;
converting the at least one electric ultra-wideband ultrasound electric signal by the at least one ultrasound transducer into at least one ultra-wideband ultrasound impact burst;
transmitting at least one ultra-wideband ultrasound impact burst into target location of the biological object;
wherein at least one transmitted ultra-wideband ultrasound impact burst comprises a low-frequency range having a duration ranging between 0.5 and 1 us at a frequency ranging between 1 MHz and 3 MHz and a high-frequency range having a duration ranging between 10 and 50 ns at a frequency higher than 3 MHz within a frequency range of frequency factor greater than 3; transmitting the high-frequency and low-frequency ranges in-phase transmitted to the biological object target area such that a period of transmitting the high-frequency range is within a period of transmitting the low-frequency range.

A further object of the invention is to provide the step of transmitting at least one ultra-wideband ultrasound impact burst in the high-frequency range transmitted in a middle of the duration of transmitting the at least one ultra-wideband ultrasound burst in the low-frequency range to the biological object target area.

A further object of the invention is to provide the step of generating at least one ultra-wideband electric signal of ultrasound frequency by the at least one ultrasound generator comprising generating a train of the ultra-wideband ultrasound impact bursts.

A further object of the invention is to provide the step of generating at least one electric ultra-wideband (UWB) electric signal of ultrasound frequency comprising consecutively generating a plurality of predetermined signals by the at least one ultrasound generator and accumulating signals consecutively generated by the at least one ultrasound generator in a buffer and synchronically transmitting the predetermined signals thereby to the at least one ultrasound transducer.

A further object of the invention is to provide the step of generating at least one ultra-wideband electric signal of ultrasound frequency comprising at least two ultra-wideband ultrasound signals at frequencies within the low-frequency and high-frequency ranges and supplementing each other; the at least two ultra-wideband ultrasound signals are band-pass filtered in the low-frequency and high-frequency ranges, amplitude normalized and synchronously summed such that a summed ultra-wideband ultrasound signal when transmitted to the at least one ultrasound transducer causes generating the at least one ultra-wideband ultrasound impact burst.

A further object of the invention is to provide the method comprising a step of illuminating the biological object area to be treated by a IR laser module; the step of illuminating the biological object to be treated further comprising sub-steps of generating an infrared laser beam by a laser light source and modulating intensity of the laser beam for generating a laser beam and modulating intensity of the laser beam to generate ultrasound with greater than 20 MHz frequency.

A further object of the invention is to provide the step of illuminating the biological object area to be treated comprising focusing a modulated laser beam on a surface of the biological object by a focusing optical element.

An aspect of some embodiments of the invention concerns a device and method for performing ultrasound therapy using ultra-wideband impact ultrasound bursts with a spectrum that is fairly uniform and fairly filled-in over a fairly wide range of frequencies, for example covering a frequency factor of 3 in in a low frequency range and at least ratio of 3 in high frequency range.

There is provided, in accordance with an exemplary embodiment of the invention, a device for generating ultra-wideband bursts of ultrasound in a body tissue, the device comprising:
a) at least one transducing element that together with a signal generating module generate the impact bursts in the body tissue using an ultrasound generation method; and
b) at least two generators together generate impact bursts having a specified intensity and spectrum at a specified target location; wherein the device is capable of generating a train of bursts at least a distance 8 cm inside a tank of distilled water from a surface of the water, having a specified intensity and a specified spectrum as a function of frequency, at least over a range of frequency covering at least frequency factor 3 in, wherein an effective spectrum which is the specified spectrum adjusted for a response of biological tissues and cells resonators at each frequency band in the range having a Q of less than 5, has 50% of its power in that range spread out over a highest power portion of the range covering at least 32% of the range, and wherein the specified spectrum goes to zero at zero frequency.

Optionally, the specified spectrum includes at least 2 consecutive such ranges of frequency where the effective spectrum has 50% of its power in that range spread out over a highest power portion of the range covering at least 32% of the range, and the specified spectrum averaged over the range of frequencies and over a duration of the train differs by less than a factor of 3 for the 2 consecutive ranges of frequency.

Optionally, the high frequency range of frequencies includes a factor of 7.

Optionally, the effective spectrum has less than 50% of its power in a range from 1 to 3 MHz spread out over a highest power portion of the range from 1 to 3 MHz, covering at least 32% of the range from 1 to 3 MHz.

Optionally, at the specified intensity the power per area per MHz is at least 5 times a thermal noise level for the specified spectrum.

Optionally, the device is capable of generating the train of bursts at least a distance 8 cm inside the tank of distilled water.

At least two ultrasound generating elements comprise a first and a second ultrasound generating element, and a signal used by the first ultrasound generating element has a lower average frequency band than a signal used by the second ultrasound generating element.

Optionally, the signal generating module is configured to generate at least one of the signals by:
a) generating a first train of ultra-wideband impact bursts;
b) modifying a shape of the spectra of bursts in the first train by filtering them with a specified filter, to produce at least a first component of the signal; and
c) using at least the first component of the signal to produce the signal.
a) generating a train of pulses;
b) modifying a shape of each pulse spectra in the first train by filtering them with a specified filter, to produce at least first and second components of the signal; and
c) using at least the first and second components of the signal to produce the signal.

Optionally, the impact bursts in train are at least approximately the set of square pulses.

Optionally, the impact bursts, after filtering, are at least approximately a sum of sinc and chirp f spectra minus frequencies 0-1 MHz.

Optionally, the signal generating module is also configured to:
a) generate—at least one train of pulses; and
b) modify a shape of the impact burst spectra in each of bursts by filtering it with a specified filter, to produce respectively components of the spectra of signals that are different in shapes from the first component of the signal; wherein using at least the first signal component to produce the signal comprises combining the first and additional signal components to produce the signal.

Optionally, in at least two of the trains of pulses, the pulse has a different frequency spectra and rate.

Optionally, the shapes of spectrum of pulses in at least two of the trains of pulses are modified by filtering them with differently acting filters.

Optionally, the signal generating module is configured to generate at least one of the signal spectra by synthesizing it digitally.

Optionally, the ultrasound generating elements comprise micromechanical transducers that generate ultrasound.

Optionally, the device also comprises a controller that controls the signal module that generates signals used by each of the ultrasound generating elements, wherein the controller comprises a memory that stores data of a treatment protocol specifying the signals used by each of the ultrasound generating elements and their timing, for at least one patient receiving treatment according to the treatment protocol.

Optionally, the treatment protocol specifies generating a train of bursts having a specified intensity, waveform and a specified spectrum as a function of frequency, at least over a range of frequency covering a factor at least of 3 in frequency in each range, wherein an effective spectrum which is the specified spectrum adjusted for a response of biological tissue and cell resonators at having an ultrasound response in each the ranges having quality factor less than 5, has 50% of its power in that range spread out over a highest power portion of the range covering at least 32% of the range, and wherein the specified spectrum goes to zero below 1 MHz Optionally, the device is a non-imaging device.

Optionally, the device is wearable, and comprises a positioning element for holding the device in a position on a body for generating the bursts in the target location body tissue. There is further provided, in accordance with an exemplary embodiment of the invention, a device for generating ultra-wideband ultrasound bursts in a form of wave packet of in a target location of body tissue, the device comprising:
a) at least one ultrasound generating elements that together with ultrasound generating module generate the impact bursts in the target location of body tissue using an ultrasound generation method; and
b) a signal generating module that generates signals used by each of the ultrasound generating elements to together generate impact bursts having a specified intensity and specified spectrum at a specified target location;
wherein the device is capable of generating a train of bursts at least a distance 8 cm inside a distilled water having a specified waveform, specified intensity and a specified spectrum as a function of frequency, at least over a range of frequency factor at least of 3 in each range of frequency, wherein an effective spectrum which is the specified spectrum adjusted for a response of tissue and cell resonators at each frequency in the range having quality factor Q less than 5, has 50% of its power in that range spread out over a highest power portion of the range covering at least 32% of the range, and wherein the specified spectrum goes to zero below 1 MHz.

Optionally, the ultrasound generating elements comprise near-infrared lasers that generate ultrasound in body tissue using optoacoustics.

There is further provided, in accordance with an exemplary embodiment of the invention, a method of ultrasound medical treatment comprising:
a) selecting a subject and target region to be treated; and
b) generating of ultra-wideband ultrasound impact bursts in a target-location of the object, having a specified intensity. Specified waveform and a specified spectrum as a function of frequency, at least over a range of frequency covering a frequency factor at least of 3 in each range in frequency, wherein an effective spectrum which is the specified spectrum adjusted for a response of biological tissue and cell resonators, at each frequency band in the range having a quality factor Q less than 5, has 50% of its power in that range spread out over a highest power portion of the range covering at least 32% of the range, and wherein the specified spectrum goes to zero below 1 MHz,

Optionally, generating the train of ultrasound impact bursts at the target location comprises using a first transducer to launch first ultrasound wave packet to the target region, from a first location on an outer surface of the object, and using a second transducer to launch second ultrasound to the target region from a second location, different from the first location, on the surface of the object, the first ultrasound waves and the second ultrasound waves combining in the target region to produce the ultrasound impact bursts having the the specified spectrum intensity as a function of frequency.

Optionally, the method comprises specifying a treatment protocol for the object, before generating the bursts of ultrasound, wherein the treatment protocol includes the specified intensity and specified spectrum, as well as a burst rate and a duration of a treatment session, and wherein generating the bursts is done according to the treatment protocol.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A schematically shows body tissue, and FIG. 1B schematically shows the interior of a cell, with resonant structures at a wide range of different distance scales and resonant frequency bands, which potentially may be excited by ultra-wideband ultrasound Impact bursts in some embodiments of the invention;

FIGS. 2A to 2D schematically show a system for generating ultra-wideband ultrasound impact bursts in body tissue, for therapy, according to an exemplary embodiment of the invention; FIG. 2A is a schematic diagram of a system configured for consecutively generating signal components, accumulating generated components in a buffer and then a consequentially transmitting accumulated components to the buffer a target location 206; FIG. 2B is a schematic diagram of a system configured for parallel transmitting signal components to a target location; FIGS. 2C and 2D are schematic diagram of the systems shown in FIGS. 2A and 2B provided with a laser module;

FIG. 3 is a flowchart showing a method of transmitting ultra-wideband ultrasound impact bursts to a target location;

FIG. 4A schematically shows a waveform of an ultra-wideband ultrasound burst, based on sinc functions that would correspond to a flat spectrum of constant frequency power density between 1 and 7 MHz, but with the waveform cut off at +0.5 microsecond, so that the burst only lasts 2 1 microseconds, and FIG. 4B schematically shows the spectrum of the waveform shown in FIG. 4A;

FIG. 5 schematically shows the spectrum of a long coherent train of bursts with the waveform shown in FIG. 4A;

FIG. 6 schematically shows a spectrum of an ultra-wideband ultrasound burst to illustrate some of the claim limitations, according to an exemplary embodiment of the invention;

FIG. 7 schematically illustrates a prior art low intensity pulsed ultrasound (LIPUS) spectrum;

FIG. 8A schematically illustrates waveforms for 7 components, each consisting of a sine wave respectively at 1, 2, 3, 4, 5, 6 and 7 MHz and lasting for an interval of 1 microsecond, that can be generated by hardware, according to an exemplary embodiment of the invention; FIG. 8B schematically illustrates a waveform that is the sum of the 7 components shown in FIG. 8A; FIG. 8C schematically illustrates the Fourier transforms of each of the components shown in FIG. 8A; FIG. 8D schematically shows the spectrum of the combined waveform shown in FIG. 8B;

FIG. 9A schematically shows a plot of an exemplary ultra-wideband ultrasound burst spectrum generated by the system of FIGS. 2A to 2D;

FIG. 9B shows a graph of dependence of the proliferation index PI on the bandwidth of the UMI Burst signal for two signal intensities 1 μW/cm² and 1 mW/cm².

FIG. 9C is a graph schematically showing the results of a study of mouse wound-healing with and without exposure to ultra-wideband ultrasound impact bursts.

FIG. 10 shows the block diagram that illustrates method of ultra-wideband micromechanical regenerative impact (UMI Burst Impact), according to an exemplary embodiment of the invention;

FIGS. 11A to 11F show examples of a complementary pair of spectra of treating signal. More particularly, FIG. 11A is a Gaussian monocycle spectrum for the frequency range 0-50 MHz; FIG. 11B presents a pulse shape corresponding to the Gaussian monocycle spectrum of FIG. 11A; FIG. 11C presents a corrective spectrum; FIG. 11D presents a corrective spectrum embodied in the form of an exponentially damped sinusoid; FIG. 11E presents a resultant corrective spectrum; and FIG. 11F presents a complex shape of treatment time-domain pulse 52 after spectrum corrector 16;

FIG. 12 shows the block diagram of a 3D system for measuring the spatial and frequency characteristics of ultra-wideband ultrasonic/micromechanical fields;

FIG. 13 illustrates the dependence of the intensity averaged over space, time and frequency of ultra-wideband micromechanical impulses with a frequency range of 1.0-7.0 MHz on the distance to the transducer, in comparison with narrow-band ultrasonic signals of 1.0, 3.0 and 5.0 MHz frequencies, and also with ultra-wideband stochastic ultrasonic noise signal with a frequency band of 1.0-3.0 MHz.

FIG. 14 illustrates the differences in the impact of ultra-wideband micromechanical bursts (UMI Bursts) with a frequency spectrum range of 1.0-7.0 MHz on the proliferation rate of normal mouse fibroblast cells, in comparison with exposure to narrow-band ultrasound with frequencies of 1.0 MHz, 3.0 MHz and 7.0 MHz.

FIG. 15 shows the differences in blast transformation (immune activity) of human T-lymphocytes in normal conditions, compared to LIPUS and UMI Burst exposures.

FIG. 16 shows the differences in dynamics of healing of extensive linear wounds of mice in normal and under LIPUS and UMI Burst influences.

FIG. 17A is a photo of a pronounced scar with traces of surgical intervention before UMI Burst procedures; FIG. 17B is a photo of the scar after 5 days of daily treatments, where the traces of sutured wound are almost invisible and the depth of the scar has decreased significantly; FIG. 17C is a photo of the scar after 9 days of applying UMI Bursts.

FIGS. 18A and 18B show a variant of the implementation of a UMI Burst method for drastic reduction of long-term dermal hyperpigmentation (dermal melasma) on the on a woman's face and for reduction of deep forehead wrinkles. FIG. 18A is a photograph taken before UMI burst therapy, and FIG. 18B is a photograph taken after the therapy.

FIG. 19 shows a variant of the implementation of the UMI Burst method in cosmetology. Reduction of deep creases of the skin on the face with quantitative control of the size of creases using the Altera 3D apparatus.

FIGS. 20A and 20B illustrate the implementation of the proposed UMI Burst method for restoring blood microcirculation in the area of the lower limb with varicose veins. FIGS. 20A and 20B are taken before and after the UMI Burst treatment.

FIG. 21 is a general block diagram of the stationary device for ultra-wideband ultrasonic micromechanical regenerative frequency burst-therapy, according to an exemplary embodiment of the invention;

FIG. 22 is a structural scheme of the personal mobile device for ultra-wideband ultrasonic micromechanical regenerative frequency burst-therapy, according to an exemplary embodiment of the invention;

FIG. 23 shows blocks and connections of the UWB volumetric piezoelectric transducer with generators, control and monitoring units;

FIG. 24 is a sensor part of the volumetric UWB transducer for ultra-wideband ultrasonic micromechanical regenerative frequency burst-therapy with the units for monitoring the presence of acoustic contact;

FIG. 25A is a graph of a main lobe of the shape of spectra of a rectangular video pulse; FIG. 25B is a graph of a time-domain waveform of a rectangular video pulse with a duration of 20 nanoseconds; FIG. 25C is a graph of the increasing part of the spectrum of a four-cycle sinusoid with a frequency of 55 MHz; FIG. 25D is a graph of a time-domain waveform image of a four-cycle sinusoid. FIG. 25E is a graph of a resultant signal obtained by summing signals shown in FIGS. 25A and 25B in the spectrum corrector;

FIG. 26 presents comparison of the spectra of own vibrations of biostructures with the spectra of known ultrasonic medical devices and the spectra of the proposed ultra-wideband micromechanical frequency devices.

### DESCRIPTION OF SPECIFIC EMBODIMENTS OF THE INVENTION

The present invention, in some embodiments thereof, relates to a device and method for medical treatment using ultrasound waves and, more particularly, but not exclusively, to promoting tissue regeneration using pulsed ultrasound at relatively low intensities.

The purpose of the present invention is to improve availability internal biological tissues for ultrasound treatment. This purpose is achieved by means of forming an ultra-wideband dual-range multicomponent wave packets containing a low-frequency spectrum and a high-frequency spectrum.

### Characteristics of Ultrasound Burst Waveform and Spectrum

An aspect of some embodiments of the invention concerns a system and method for producing therapeutic ultrasound bursts in the body, with a spectrum that is relatively uniform over at least one relatively broad range of frequencies. These bursts are sometimes referred to herein as ultra-wideband bursts (UWB), or as Ultra-wideband Micromechanical Impact bursts (UMI). These terms are sometimes used interchangeably. “UWB” is a known term of the art that is often used when referring to ultrasound bursts used for ultrasound imaging. “UMI” is not a standard term, and is used here to emphasize the therapeutic impact that the bursts may have.

For example, the spectrum of the bursts covers a frequency range of at least a factor of 3, or at least a factor of 4, or at least a factor of 10, and its relative uniformity within this frequency range is defined as follows. First, instead of directly analyzing the spectrum S (@), where w is the frequency, the analysis is optionally done on an effective spectrum Seff (@), which represents the response to the actual spectrum by resonant structures in body tissue that have a quality factor Q, as will be described below. The effective spectrum is given by

$${S}_{eff} ( ω ) = \frac{1}{2 π Q} \int d Δω S ( ω + Δω ) \frac{ω}{( {( Δω )}^{2} + {ω}^{2} / 4 {Q}^{2} )}$$

Here Q is taken to be the highest Q that is likely to be found among the resonant structures that the bursts are exciting in body tissue, for example Q=5. Alternatively Q may be taken as 4 or 3 or 2.5, for example, or any reasonably low value. Optionally, different values of Q may be assumed for different frequencies or different frequency ranges. A potential advantage of analyzing the effective spectrum is that there may be many rather different-looking actual spectra that have the same or nearly the same effective spectrum, and all of them are expected to have nearly the same biological effect on body tissue. So in some embodiments of the invention, “relatively uniform over a relatively broad range of frequencies” is taken to depend only on characteristics of the effective spectrum. Another potential advantage of analyzing the effective spectrum is that the effective spectra tend to have a similar typical appearance even for actual spectra that look rather different, because the effective spectra are all fairly smooth. This may mean that if a certain criterion is developed for defining when an effective spectrum is “relatively uniform over a relatively broad range of frequencies,” and the criterion is found to work well for a few different spectra that might be used, then it is likely to work well for other spectra that might be used, because their effective spectra will not very different.

“Relatively uniform over a relatively broad range of frequencies” may be defined in terms what fraction of the total power over the whole range of frequencies is found in the highest power portion of what range of frequencies. For example, if 50% of the power is found in a highest power portion of the range that only covers a small fraction of the whole range, much less than 50%, that means that the effective spectrum is not very uniform over the whole range. But if 50% of the power is spread out over a highest power portion of the range that covers a larger fraction of the range, that means that the effective spectrum is relatively uniform over the range. Optionally, the criterion for “relatively uniform over a relatively broad range of frequencies” is that, for some range of frequencies converting a factor of 3, 50% of the total power in the range is found in a highest power portion of the range that is at least 32% of the range. Alternatively, the criterion is that 50% of the total power is found in a highest power portion of the range that is at least 25% of the range, or at least 27%, or at least 30%, or at least 35%.

Alternatively, the criterion is based on what fraction of the total power is found in the highest power portion of the range that covers 50% of the range. The higher the fraction of total power that is found in the highest power 50% of the range, the less uniformly distributed the effective spectrum is considered to be. For example, the criterion for “relatively uniform over a relatively broad range of frequencies” is that, for at least one range of frequencies that covers a ratio of 3 in frequency, no more than 70% of the total power is found in the highest power portion of the range that covers 50% of the range. Alternatively, the criterion is that no more than 60% of the power is found in the highest power 50% of the range, or no more than 63%, or not more than 65%, or no more than 67%, or no more than 72%, or no more than 75%, or no more than 80%.

Alternatively, the criterion for “relatively uniform over a relatively broad range of frequencies is based on the fraction of total power that is found in a different highest power fraction of the range, for example in 20% or 30% or 40% or 60% or 70% or 80% of the range, or it is based on the highest power portion of the range that has a different fraction of the total power, for example 20% or 30% or 40% or 60% or 70% or 80% of the total power.

A convenient way to calculate these numbers is to calculate the effective spectrum in a spreadsheet such as Excel, at a set of equally spaced points over the range of frequencies, as a column of values. The column of values is then sorted from the highest to the lowest value, and another column is created showing the sum of the values from the top of the sorted column to each point in the middle of the sorted column. The sums of values are then normalized by the value at the bottom of the sorted column, which represents the total power integrated over the range of frequencies. The normalized value halfway down the column then represents the fraction of power found in the highest power 50% of the range of frequencies, and similarly for other fractions of the range of frequencies. To find the highest power fraction of the range in which 50% of the power is found, look for the entry in entry in the column which is 0.500, interpolating if necessary. The fraction of the range in which 50% of the value is found will be the fraction of the distance down the column at which 0.500 appears in the normalized sum of values, and similarly for other fractions of the total power.

Another way to characterize the degree to which a spectrum is relatively uniform over a relatively broad range of frequencies is to compare the standard deviation of values of the effective spectrum over the range of frequencies, to the mean value of the effective spectrum over the range of frequencies. Here the mean value of the effective spectrum is given by

$${S}_{mean} = \frac{1}{{ω}_{max} - {ω}_{min}} {\int }_{{ω}_{min}}^{{ω}_{max}} d ω {S}_{eff} ( ω )$$

where ωₘᵢₙ and ωₘₐₓ are the minimum and maximum frequencies of the range, and the standard deviation of values of the effective spectrum is given by

$${S}_{sd} = {[ \frac{{\int }_{{ω}_{min}}^{{ω}_{max}} d {ω [ {S}_{eff} ( ω ) - {S}_{mean} ]}^{2}}{{ω}_{max} - {ω}_{min}} ]}^{1 / 2}$$

The smaller the ratio of standard deviation to mean value, the more uniform the effective spectrum is over its range, according to this definition. For example, the spectrum may be considered relatively uniform over a relatively broad range of frequencies if, for at least one range of frequencies covering a factor of 3, the ratio of the standard deviation to the mean is less than 0.50. Alternatively, the ratio is required to be less than 0.35, or less than 0.40, or less than 0.45, or less than 0.55, or less than 0.60, or less than 0.65.

Optionally, the spectrum goes to zero at zero frequency, and optionally it increases with frequency as the square of the frequency or a higher power of frequency, in the limit of low frequency. If the spectrum does not go to zero at zero frequency, but is flat at a constant value near zero frequency, then there will always be a range of frequencies covering a factor of 3, near zero frequency, where the spectrum is very uniform. That is true, for example, of gaussian pulses, which have a gaussian spectrum that is constant around zero frequency. Even if the spectrum goes to zero at zero frequency, but only linearly with frequency, there will be ranges of frequency, covering a ratio of 3, that are in this linear regime, and in those ranges, depending on how “relatively uniform over a relatively broad range of frequencies” is defined, such a spectrum may also always meet that definition, regardless of its behavior at higher frequencies. Requiring that the spectrum goes to zero at least quadratically at low frequencies excludes such spectra that meet the criterion for “relatively uniform over a relatively broad range of frequencies” for trivial reasons. In practice this is likely to be true of ultrasound bursts generated by types of transducers, such as PDVF transducers, that have a poor response at low frequencies, for example below 1 MHz.

It should be noted that these definitions of “relatively uniform over a relatively broad range of frequencies” only require that there is some range of frequencies, covering a factor of 3, where the criterion is satisfied. There is no requirement that the range of frequencies include the highest power part of the ultrasound spectrum. By these definitions, if ultra-wideband bursts of ultrasound are used together with narrowband ultrasound of much higher power at a very different frequency, then the total ultrasound spectrum is still considered to be “relatively uniform over a relatively broad range of frequencies.” It is possible that lower power ultra-wideband bursts, used together with higher power narrowband ultrasound at a very different frequency, will still provide the therapeutic benefits of ultra-wideband bursts, in spite of the presence of the narrowband ultrasound, so this case is not excluded from the definition of “relatively uniform over a relatively broad range of frequencies.” Alternatively, the range of frequencies has to include the highest power ultrasound frequencies present, and that case is excluded from the definition.

Such an ultra-wideband ultrasound burst spectrum differs from the spectrum of prior art ultrasound bursts used for therapy, which are narrowband, for example having a characteristic bandwidth that is at least 3 times or 5 times or 10 times narrower than the peak frequency. Or, as is often the case for LIPUS (low intensity pulsed ultrasound) therapy, the spectrum consists of a few narrowband frequencies that are well separated, for example at a fundamental frequency ω₀ and at harmonic frequencies such as 3ω₀ and 5ω₀, each with a characteristic bandwidth that is well below ω₀, for example less than 0.5ω₀ or less than 0.2ω₀. It should be noted that whether or not a given burst spectrum satisfies these conditions does not depend on fine details of the structure of the spectrum, but only on characteristics of the effective spectrum, which has these fine details smoothed out.

Optionally, these conditions on uniformity of the intensity over the range of frequencies apply not to the intensity of the ultrasound at the transducers producing the ultrasound at the outer surface of the body, but at a target location inside the body, where the therapy is being applied. The spectrum may differ at these locations, because higher frequency components of ultrasound tend to be more attenuated than lower frequency components when propagating through body tissue.

Optionally, the bursts are about as short as they can be, given the shape of their spectrum. For example, if most of the integrated intensity of the spectrum, or more than 75% of the integrated intensity of the spectrum, occurs at a frequency greater than ωₘᵢₙ, then most of the power of the burst occurs within a duration of no more than 3/ωₘᵢₙ or no more than 5/ωₘᵢₙ, or no more than 10/ω, or no more than 20/ωₘᵢₙ. Although the burst spectrum, being ultra-wideband, includes significant contributions from frequencies well above ω, the components at those widely different frequencies overlap substantially in time, so that the effective duration of the burst is not much more than, and may even be less than, a wave period at the lowest frequency that contributes significantly to the burst spectrum. Having the bursts be about as short as they can be, given the shape of the spectrum, has the potential advantage that the ultrasound may have a greater biological effect on the tissue, and/or may produce less heating of the tissue, than if the bursts were longer. This may be true, for example, because shorter bursts may have a higher repetition rate, and/or may have a greater instantaneous power per area relative to the time-averaged power per area which determines the heating rate. Alternatively, the duration of the burst is much greater than the minimum it can be, given the shape of the spectrum.

### Possible Mechanism of Operation

The inventors believe that ultra-wideband ultrasound bursts that satisfy these conditions can be therapeutically effective in certain situations, for example in promoting wound healing, or in promoting the proliferation of certain types of cells, such as fibroblasts or hematopoetic stem cells, that are associated with wound healing and other regenerative processes in the body. Without limiting ourselves to any one theory of the reasons for these effects, the following model may have some validity. It is known that body tissue contains a large number of resonant micromechanical structures, that have a large range of different characteristic sizes, and a large range of resonant frequencies, for example ranging from about 1 MHz to about 100 MHz or even 250 MHz. For example, at the largest distance scale and lowest resonant frequencies, roughly 1 to 10 MHz, there are macroscopic regions of tissue including cellular ensembles and the extracellular matrix. At smaller size scales and higher resonant frequencies, for example between 5 and 20 MHz, there are individual cells and cell membranes. At still smaller size scales and higher resonant frequencies, there are organelles, cytoskeletons, and other structures within the cell, and there is the cell nucleus, including the nuclear membrane, the structure of nuclear proteins and the chromatin. At still smaller sizes and higher resonant frequencies, up to 100 MHz or even 250 MHz, there are macromolecular condensates within the cell and within the nucleus, and DNA molecules. In the normal operation of the cell, these mechanical structures may act as sensors, that respond to external stimuli, such as a force pushing the outside of a cell, and initiate a pathway of biochemical transformations, like an avalanche, for example ultimately activating certain genes, that allow the cell to respond to the stimuli in a way that promotes the survival of the organism. Ultrasound bursts with a given frequency component may excite structures that are resonant at that frequency, and initiate such a pathway of activity. Ultrasound bursts can also energize structures that are primarily chemical sensors.

At first it might seem that, unless the details of these resonant mechanical structures, their resonant frequencies and the pathways of activity that they may initiate, are fully understood, it might not be possible to achieve specific therapeutic goals by bombarding body tissues with ultra-wideband ultrasound bursts. However, it seems possible that when a body part suffers from a traumatic injury, many different mechanical structures, with a wide range of resonant frequencies, are all excited by the trauma that caused the injury. Consequently, the machinery of the cell may have evolved to activate pathways that promote healing, such as causing the proliferation of fibroblasts and stem cells, whenever a large number of different resonant structures, with a wide range of different resonant frequencies, are excited. If this is so, then it may be possible to activate those healing pathways, even without understanding the full details of how they work, just by exposing body tissue to ultrasound waves at frequencies that densely fill a fairly broad range of frequencies, with a spectrum that is fairly uniform, rather than dominated by only one or a few frequencies. Potentially, ultra-wideband ultrasound bursts, applied to the right tissue, could be useful for producing any therapeutic regenerative effect that could be associated with stem cells, including wound-healing, healing of broken bones, treating stroke, myocardial infarction, atherosclerosis or ischemia, repairing spinal cord injury, repairing the optic nerve and the retina, treating glaucoma, treating diseases of the skeletomuscular system, treating diabetes, some types of cancer, skin conditions, and degenerative diseases of the brain. The ultra-wideband ultrasound impact bursts might have such an effect even if the healing pathways normally activated by the body are not sufficiently activated by the trauma or other condition that is being treated. The ultrasound impact bursts might be able to initiate activation of such a pathway, which might continue to function properly on its own once it has been jump-started by the ultrasound impact bursts.

It should be noted that these resonant mechanical structures are expected to have fairly low Q, for example less than 5, or less than 4, or less than 3, or less than 2.5, because body tissue, and the interior of cells, is generally soft and viscous. For that reason, the response of these resonant mechanical structures, and any consequent therapeutic effect, is expected not to depend on fine details of the spectrum of the ultrasound, but only on the effective spectrum, for which intensity values are averaged over ranges of frequency that differ by at least 20%.

### Ranges of Frequency and Intensity

Optionally, the ultra-wideband ultrasound bursts have spectra with significant contributions extending over much more than a factor of 3 in frequency. For example, the spectrum has fairly uniform intensity, when averaged over frequency bands of factors of at least 1.2, over a range of frequencies covering a ratio of between 3 and 5, or between 5 and 10, or between 10 and 20, or between 20 and 50, or between 50 and 100, or more than 100. For example, the range of frequency is about 1 MHz to 7 MHz, or about 3 MHz to 10 MHz, or about 7 MHz to 20 MHz, or about 1 MHz to 20 MHz, or about 1 MHz to 50 MHz, or about 1 MHz to 100 MHz, or about 1 MHz to 250 MHz. Within most of this range the intensity is optionally uniform (averaging within each of consecutive bands of factors of 1.2 in frequency) 10 dB, o, optionally with the exception of a small number of bands of a frequency of a factor of 1.2 where the intensity is lower. Optionally, there are a plurality of consecutive ranges of frequency of a factor of 3, each of which satisfy one of the conditions given above, and adjacent ranges have the same average frequency within 10 dB.

Optionally, the power per area per MHz during an ultrasound burst, at a given frequency or averaged over a given range of frequencies, is at least 3 times the thermal noise level, or at least 5 times the thermal noise level, or at least 10 times the thermal noise level, or at least 20 times the thermal noise level, or at least 50 times the thermal noise level. The thermal noise level of ultrasound waves, in power per area per MHz, may be given by π₂k_{B}Tω²/cₛ, where k_{B}=1.38×10⁻²³ m² kgs₋₂K⁻¹ is Boltzmann's constant, T is the temperature, which is 310 K in the human body, w is the frequency in radians per second, and c s is the sound speed, which is about 1.5×10³ m/s in soft tissue. Depending on how the thermal noise level is defined, the numerical factor of π² in the above expression may be different. The thermal noise level at 1 MHz, ω=2π×10⁶ s⁻¹, is 6×10⁻⁸ W/cm² MHz, according to this expression, and increases in proportion to the square of the frequency at higher frequencies. We expect that ultrasound bursts that have intensities that are less than the noise level at all frequencies will not have any therapeutic effect. But it should be noted that this refers to the intensity during a burst. If the duty cycle of bursts is much less than 1, then the time-averaged power per area per MHz, which is what is usually measured, will be reduced by the duty cycle, and may be well below the thermal noise level and still have a therapeutic effect. For example, the inventors have found that a time-averaged intensity of 1 μW/cm², integrated over the spectrum of the burst, which might be uniformly distributed between 1 and 20 MHz, can have a strong therapeutic effect, when the duty cycle is less than 1%. Optionally, the time-averaged power per area per MHz, integrated over the spectrum of the burst, for example for a uniform distribution 1 between 1 and 20 MHz, is between 1 μW/cm² and 10 μW/cm², or between 10 μW/cm² and 0.1 mW/cm², or between 0.1 mW/cm² and 1 mW/cm², or between 1 mW/cm² and 10 mW/cm², or between 10 mW/cm² and 0.1 W/cm², or between 0.1 W/cm² and 1 W/cm², or greater than 1 W/cm². Optionally, the duty cycle is greater than 10%, or between 1% and 10%, or between 10⁻³ and 1%, or between 10⁻⁴ and 10⁻³, or less than 10⁻⁴. Optionally, the burst repetition rate is less than 100 Hz, or between 100 Hz and 1 kHz, or between 1 kHz and kHz, or between 10 kHz and 100 kHz, or more than 100 kHz. For a burst spectrum that has a minimum frequency of 1 MHz that makes a substantial contribution to the spectrum, a burst of minimum duration will have a duration of about 1 μs.

Optionally, these powers per area refer to the power per area produced by a transducer at the surface of the body. Alternatively, these powers per area refer to the power per area produced at a target region some distance beneath the surface of the body. For example, the target region is less than 5 mm deep, or between 5 mm and 1 cm deep, or between 1 and 2 cm deep, or between 2 and 4 cm deep, or between 4 and 8 cm deep, or between 6 and 10 cm deep, or more than 10 cm deep. Optionally, these powers per area refer to the power per area produced for example in a phantom, or in a tank of water, or in any other device used for calibrating and/or characterizing ultrasound systems. Such devices can also be used to calculate or estimate what ultrasound waveform and intensity should be used at the transducer, or at an optoacoustic ultrasound generator, to produce a specified waveform and intensity at a target region at a given location inside a patient's body, and to determine whether a given ultrasound system is capable of generating ultrasound bursts of a specified intensity and waveform at a target region at a given location inside a patient's body.

### Treatment Protocols

Treating different medical conditions, and/or different patients, may require different treatment protocols. For example, ultrasound bursts of a specified waveform, spectrum, intensity and repetition rate may be applied to target region for a specified treatment duration. Treatment may be repeated periodically, for example once a day, for a specified number of treatment sessions, for example 5 or 10. The number of treatment sessions, and other aspects of the treatment protocol, may be adjusted during the course of treatment, in response to observed changes in the condition of the patient. The treatment protocol may be stored in the memory of the device, which may be particularly useful if the patient is being treated at home with a portable device.

### Methods of Generating Ultrasound

Optionally, the ultrasound is generated at the surface of the body, by an electro mechanical transducer, for example a ceramic transducer or a PVDF transducer. Alternatively, the ultrasound is generated by a transducer introduced into the body by probe, for example through a natural orifice of the body, for example using a catheter, or may be used on an inside surface of the body during surgery. Alternatively, the ultrasound is generated inside the body by optoacoustic effect, for example at or near the target region, by a laser beam, for example a near IR laser beam that can penetrate even several centimeters into body tissue, that is modulated at the desired ultrasound frequency, and that generates the ultrasound by optoacoustics. Optionally, especially if the ultrasound burst has a wide frequency range, two or more different transducers are used, each transducer covering a different frequency range that it is best suited for, or there are one or more mechanical transducers plus an optoacoustic laser beam, each generating ultrasound at a different range of frequencies. Generally, optoacoustics is especially suited for generating ultrasound at higher frequencies, for example above 20 MHz or above 50 MHz, because ultrasound of such high frequencies does not penetrate very far into body tissue. At lower frequencies, it is known that certain types of mechanical transducers are better suited for generating ultrasound at certain frequency ranges, for example ceramic transducers generally work best at lower frequencies, below about 10 MHz, while PVDF transducers generally work best between about 1 MHz and 30 MHz.

Optionally, the ultrasound burst is generated by generating a voltage signal of the desired waveform, amplifying the voltage signal, and applying the amplified voltage signal to the transducer or to the optoacoustic ultrasound generator. Optionally, more than one voltage signal is used, each covering a different range of frequencies and each applied to a different transducer, or optoacoustic ultrasound generator. Optionally, the voltage signals are synthesized digitally, for example using a modified mathematical expression, such as a Sinc and Chirp functions. However, it becomes increasingly difficult to digitally synthesize a voltage signal at frequencies greater than 20 MHz. Especially for higher frequencies, it may be more practical to generate the voltage signal by hardware. For example, one or more square pulses are generated, and/or a sine wave segment of a certain duration, and/or a damped sine wave segment of a certain duration, and/or short pulses of different shapes. These signals are optionally passed through a different filter or set of filters for each signal, and the filtered signals are added together to produce the waveform function. For example, a set of seven segments of sine waves, each 1 microsecond long, respectively at 1, 2, 3, 4, 5, 6 and 7 MHz, can be combined to produce a burst that has a spectrum that is approximately flat between 1 and 7 MHz, and falls off substantially outside this range.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

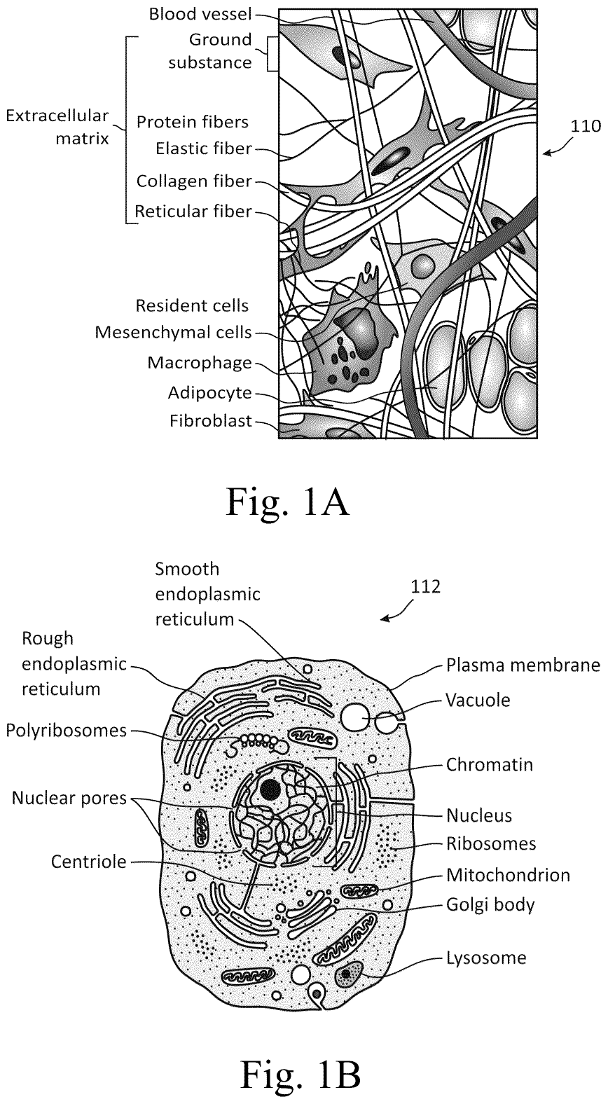

Referring now to the drawings, FIGS. 1A and 1B schematically illustrate resonant structures in body tissue, or inside cells, that an ultra-wideband ultrasound burst can excite. FIG. 1A shows a region 100 of body tissue, including an extracellular matrix, with blood vessels such as capillaries running through it, and resident cells such as mesenchymal cells, macrophages, adipocytes, and fibroblasts embedded in it. The extracellular matrix includes, for example, ground substance, and protein fibers including elastic fibers, collagen fibers, and reticular fibers. These components of the tissue, individually or collectively, can have resonance frequencies, generally with low Q such as Q≤5, and at relatively low frequencies, for example between 1 MHz and 3 MHz, and/or between 3 MHz and 10 MHz, and can respond to ultra-wideband ultrasound bursts that include components at those frequencies. The resonant structures here may be larger than single cells.

FIG. 1B shows a single cell 102, such as one of the cells resident in the extracellular matrix shown in FIG. 1A, containing internal structures of a wide range of sizes, that may have resonant frequencies that are found in the spectra of ultra-wideband ultrasound bursts. The cell as a whole may also have a resonant frequency that responds to ultra-wideband ultrasound bursts. The internal structures shown in FIG. 1B include the plasma membrane surrounding the cell, vacuoles, the endoplasmic reticulum, Golgi bodies, the nucleus, lysosomes, mitochondria, the centriole, and, much smaller, ribosomes, and macromolecular condensates, which are represented for example by the small dots scattered throughout the cytoplasm and the nucleus. Transport channels in the plasma membrane may also be considered macromolecular condensates. Other intracellular structures, not specifically shown in FIG. 1B, include the cytoskeleton and its components. The nucleus has its own internal structures, including the nuclear membrane with its own pores, the nucleosome (represented by a large black dot in the nucleus, and the chromatin structure, including nucleoproteins and DNA molecules. These structures, depending on their sizes, may have resonant frequencies ranging from about 10 MHz up to 100 MHz or even 250 MHz. Like the larger resonant structures shown in FIG. 1 A, the intracellular structures shown here generally have rather low Q, for example less than 5.

### Description of System

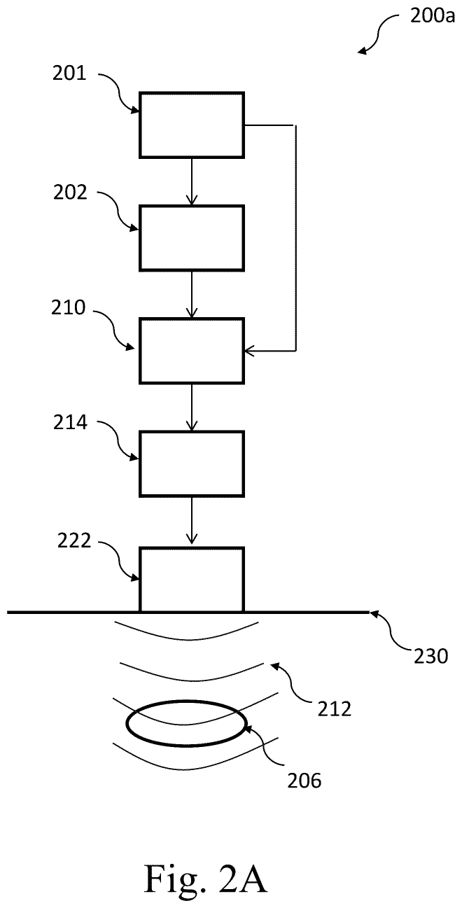

FIGS. 2A to 2D present four alternative embodiments 200a to 200d of a therapeutic device for applying ultra-wideband ultrasound impact bursts to biological object 206.

Referring to FIG. 2A, therapeutic device 200a comprises at least one ultrasound controller 201 configured for controlling at least one ultrasound generator 202 configured for generating ultra-wideband ultrasound electric signals, which via band-pass filter 202 are fed to buffer 210. According to embodiment 200a, generator 202 consecutively generates a number of signal components which are accumulated in buffer 210 to form an ultra-wideband ultrasound impact burst. After accumulating all components of the ultra-wideband ultrasound impact bursts, they are in-phase fed to amplifier 214 and further to transducer 222 which transmits ultra-wideband ultrasound impact burst 212 in the direction of a target area location within biological object 206.

According to the present invention, transmitted ultra-wideband ultrasound impact burst 212 comprises a low-frequency range having a duration ranging between 0.5 and 1 μs at a frequency range between 1 MHz and 3 MHz and a high-frequency range having a duration ranging between 10 and 50 ns, within a frequency range of a frequency factor greater than 3.

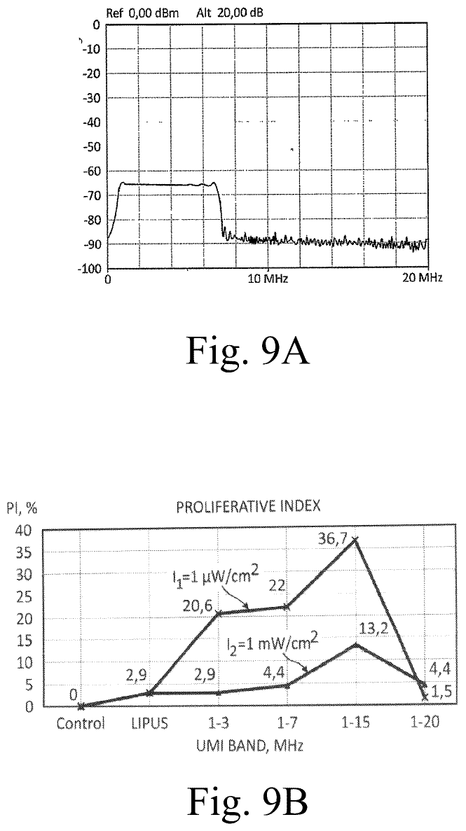

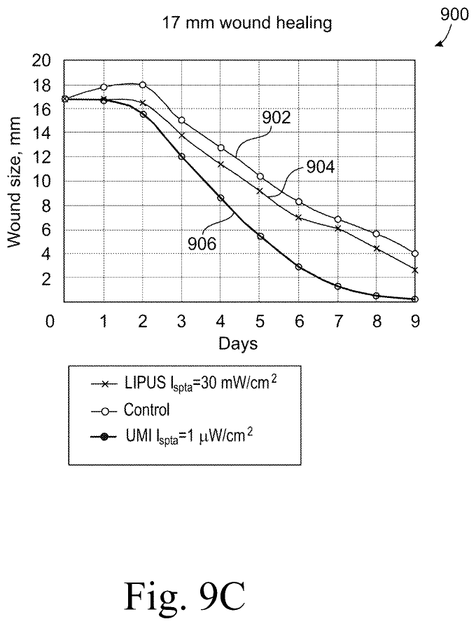

It was experimentally shown (see FIGS. 12 and 13) that attenuation of transmitted ultra-wideband ultrasound impact burst (curve 78 in FIG. 13) containing a combination of low-frequency and high frequency spectra is significantly lower than the ultrasound mono-frequency pulses.

Referring to FIG. 2B, therapeutic device 200b comprises at least one ultrasound controller 201 configured for controlling at least two ultrasound generator 202 and 204 which are in turn configured for generating ultra-wideband u electric signa ultrasound frequency components. The signal components via bandpass filter 210 are fed to signal corrector 213 which is configured for synchronously and in-phase summing the at least one ultrasound signal in the low-frequency range and at least one ultrasound signal in the high-frequency range as specified above.

Referring to FIG. 2C, therapeutic device 200c comprises a combination of device 200a and laser module 240 which further comprises laser 241 (for example a laser diode), an electro-optical modulator 242 configured for high frequency modulation up to 30 MHz and focusing optical element configured for concentration of laser radiation 246 inside body 230. Modulated laser radiation propagates within body 230 in direction of target location 206 and creates acoustic impact bursts wave packet of the carried frequency spectra.

Referring to FIG. 2D, therapeutic device 200d comprises a combination of device 200b and laser module 240. Therapeutic devices 200a, 200b, 200c, and 200d are equally I the scope of the present invention.

Each ultrasound generating element, when there is more than one of them, driven by its own amplified signal, produces a component of the ultrasound, and the components combine to form ultrasound bursts of a specified waveform and spectrum in the target region. Optionally, the different ultrasound generating elements are located at different locations on the surface of the body. A potential advantage of using that configuration is that unwanted effects outside the target region may be reduced or eliminated because ultrasound waves from different generating elements only overlap in the target region. Having different ultrasound generating elements at different locations may also allow steering or focusing of ultrasound wave packet on the target region,

Optionally, the signals used by the different ultrasound generating elements have different frequency content, for example with one ultrasound generating element better suited for producing ultrasound of higher frequencies in the target region, and the other ultrasound generating element better suited for producing ultrasound of lower frequencies in the target region. Optionally, the ultrasound generating elements are mechanical transducers, for example using piezoelectric ceramics, or PVDF, that produce ultrasound bursts that propagate into the body from a location on the surface of the body. Piezoelectric ceramic transducers, for example, cone transducers, are generally more suitable to generating lower frequency ultrasound, for example below 10 MHz, while PVDF transducers are generally more suitable to generating higher frequency ultrasound, for example above 3 MHz. When PVDF transducers or other piezoelectric transducers are used as ultrasound generating elements, then the transducer has two electrodes, for example a front electrode facing the body and a rear electrode facing away from the body, and the voltage output of the amplifier for that transducer is applied across the two electrodes. Any known ultra-wideband ultrasound transducer may be used, that is capable of operating in the desired frequency range, and that has a suitable acoustic impedance for transmitting ultrasound bursts into body. An example of a suitable ultrasound signal generator is the SigLent SDG 6052X DDS generator from SigLent Technologies Co., with a multimode ultra-wideband transducer head with a diameter of 20 mm, mentioned below as being used in some tests done by the inventors. A suitable transducer is described, for example, in Russian patent RU 2066215 C1, and in Ostrovsky and Marchenko, J. Ultrasound Med. 11(3), 557 (1992).

Alternatively, at least one e of the ultrasound generating elements is in an optoacoustic generating element, which generates ultrasound inside the body, for example directly in the target region or near the target region, by using a laser beam, such as a near infrared laser beam that can penetrate several centimeters into body tissue, that is modulated at the ultrasound frequency, and produces pressure perturbations in the body tissue at the modulation frequency by heating of the body tissue. Optoacoustic generation of ultrasound may be especially suited for the highest ultrasound frequencies, for example above 20 MHz or above 50 MHz, because such high frequency ultrasound does not penetrate very far through body tissue, and may be best generated inside the body near the target region.

Optionally, controller 201 comprises a memory that stores information about the treatment protocol that the patient is receiving for particular medical condition, including ultrasound parameters such as the waveform, spectrum, intensity, burst repetition rate, and duration of each treatment session. Optionally the patient receives the treatment at home, not directly supervised by medical personnel, and the treatment protocol, stored in memory, is applied automatically for each treatment session.

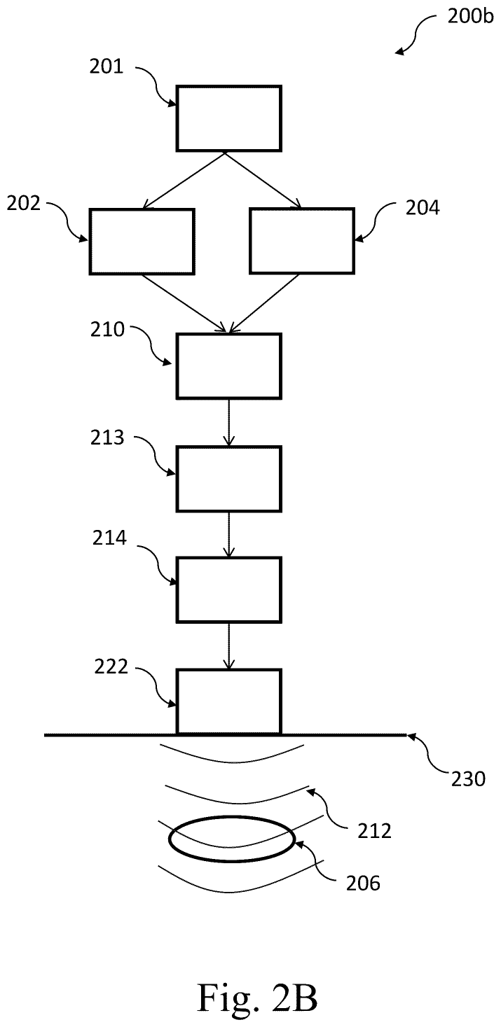

FIG. 3 shows a flowchart 300 for a method of applying ultra-wideband ultrasound impact bursts to a biological object, for example using the system of FIGS. 2A to 2D. At step 302, the therapeutic device is provided. A transducer is attached to the biological object in proximity with an object area (target area) to be treated (step 304). After that, under a command of a controller, at least one electric ultra-wideband signal of ultrasound frequency having a low-frequency and high-frequency ranges is generated by at least one ultrasound generator (step 306). The transducer converts at least one ultra-wideband electric signal by into at least one ultra-wideband ultrasound impact burst (step 308). and in-phase transmits the low-frequency range and a high-frequency ranges of the at least one ultra-wideband ultrasound impact burst to the target area in a into the biological object (step 310).

The low-frequency range portion of the ultra-wideband ultrasound impact burst is characterized by a duration ranging between 0.5 and 1 μsec at a frequency ranging between 1 MHz and 3 MHz, while the high-frequency range portion has a duration ranging between 10 and 50 ns at a frequency higher than 3 MHz within a frequency range of a frequency factor greater than 3. The high-frequency and low-frequency ranges of the ultra-wideband ultrasound impact burst are in-phase transmitted to the target location of the biological object such that a period of transmitting said high-frequency range is within a period of transmitting said low-frequency range.

If the ultrasound generating elements are elements of a phased transducer array, then the voltage signals for different elements of the array may be identical except for phase differences that vary linearly across the array to control the direction of ultrasound burst that it produces. The controller, controlling the ultrasonic frequency burst generator, can optionally generate a different signal for different ultrasound generating elements, with a spectrum of frequencies suitable for that ultrasound generating element. Optionally, the generator generates the waveform voltage signals needed to produce ultrasound bursts of a specified intensity and waveform at a specified target region in an object, for example calculated in advance, or calculated in real time by the controller, according to a model of ultrasound propagation in the biological object.

### Exemplary Burst Characteristics

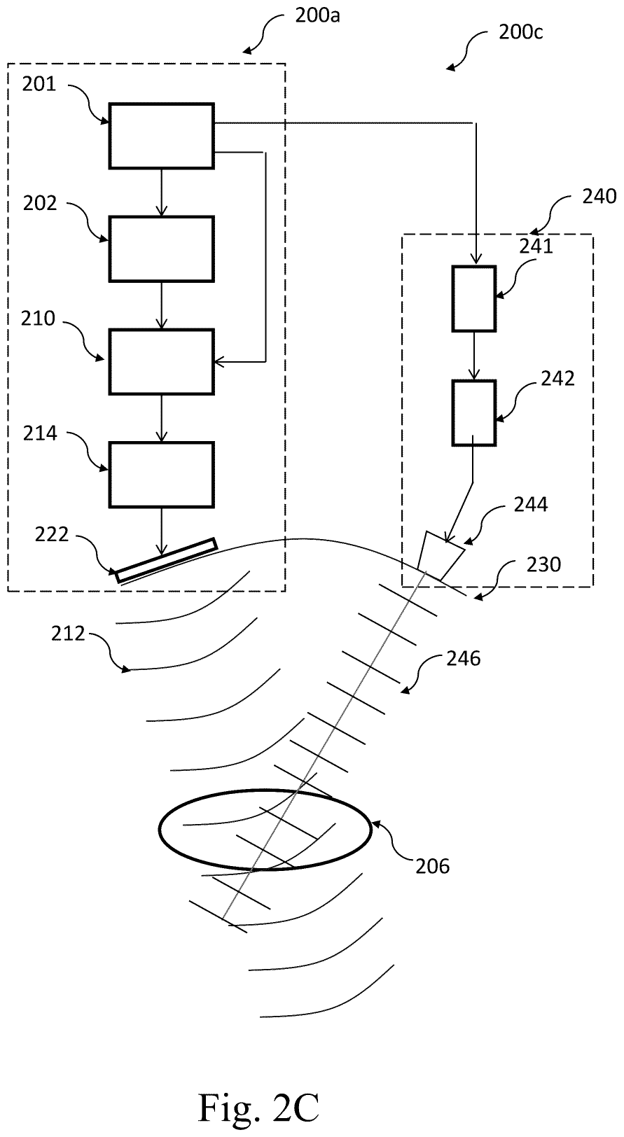

FIG. 4A shows a plot 400 of a waveform of an ultrasound burst that has an amplitude A as a function of time t given by

$$A ( t ) = \frac{sin ( 7 {ω}_{0} t ) - sin ( {ω}_{0} t )}{{ω}_{0} t} for \vert {ω}_{0} t \vert < 2 π$$

$$A ( t ) = 0 for \vert {ω}_{0} t \vert > 2 π$$

This waveform was chosen because, if the equation at the top is extended to all values of t, then theoretically it will have a spectrum that is constant between ω₀ and 7ω₀, for example, between 1 MHz and 7 MHz, and zero everywhere else. Such a spectrum is an example of a spectrum that is expected to be useful for therapeutic ultra-wideband ultrasound bursts, because it can excite a lot of different resonant frequencies of structures found in tissues and cells. In fact, it is impossible to generate a burst that lasts an infinite amount of time, and in practice it is necessary to cut the burst off at some finite time. In this case, the waveform was set to 0 at all times beyond ±2π/ω₀, which is the second zero of the term sin (ω₀t)/(ω₀t), and the burst has a duration of 2 microseconds if ω₀=2×10⁶ radians per second, the frequency of a 1 MHz wave. In principle, the controller could digitally synthesize the voltage signal shown in FIG. 4A, in order to generate such a burst.

FIG. 4B shows a spectrum 402 calculated numerically, using the FFT function in Excel, for waveform 400 in FIG. 4A. Spectrum 402 is similar to the spectrum expected for an infinitely long burst of the form given by the equation above, constant between ω₀ and 7ω₀, which may be thought of as between 1 MHz and 7 MHz, and zero everywhere else. However, due to the fact that the waveform was cut off at |ω₀t|>2π, rather than extending to infinity, the spectrum has some wiggles between ω₀ and 7ω₀, and is slightly above 0 even at frequencies above 7Ω₀ and below ω₀. It is expected that these small differences between the actual spectrum 402 and the ideal spectrum would not have any noticeable effect on the biological effects that the ultrasound bursts would have.

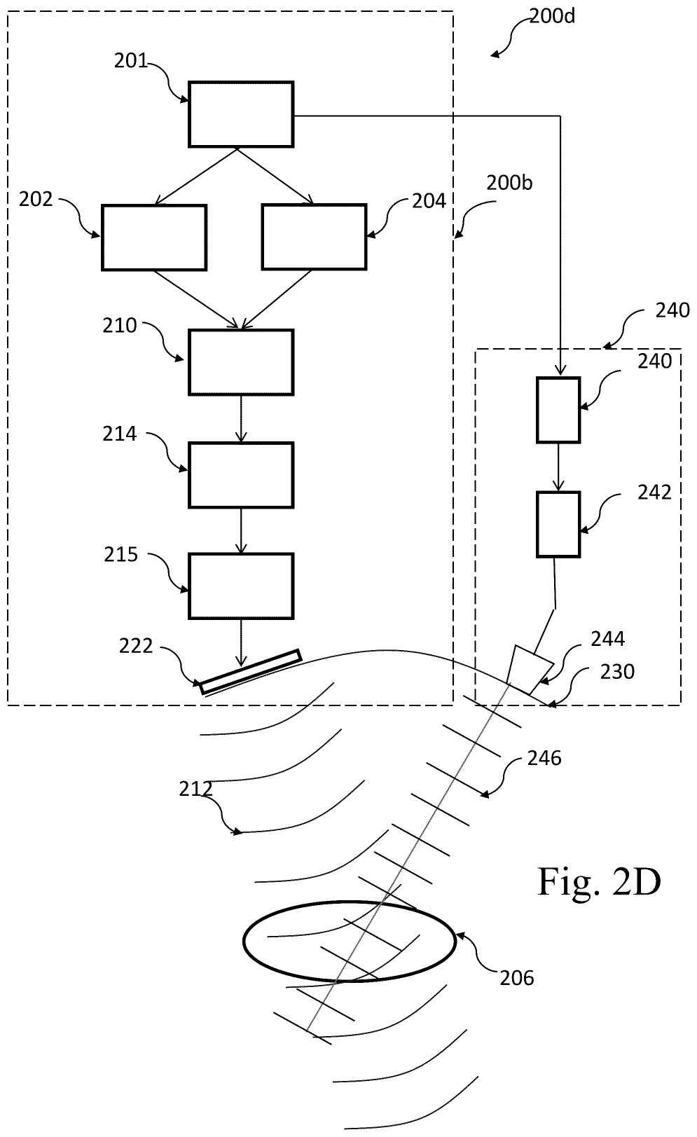

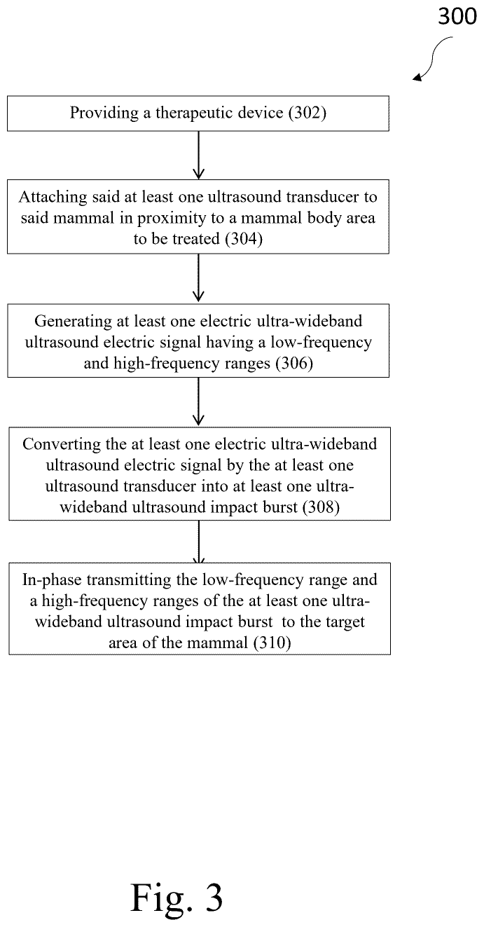

FIG. 5 shows the spectrum 500 that would be obtained for an infinite coherent train of bursts each with the waveform given by 400, author's suppose 1 microsecond. The continuous spectrum 402 is replaced by a series of δ-functions, located at integer multiples of 0.25ω₀, where the value of each δ-function integrated locally over frequency is proportional to the height of the spectrum 402 at that frequency. The δ-functions shown in plot 500 should really be infinitely high and infinitesimally thin, but the plot is drawn with the height of each spike proportional to the integrated power in that 8-function. Although the detailed appearance of spectrum 500 looks quite different from the appearance of spectrum 402, in fact the biological effects of the impact bursts are expected to be virtually identical in both cases, at least for many of the structures being excited. That is because the damping time of the resonant structures that the bursts could be exciting is expected to be much less than 1 microseconds, in fact less than about 5/2π microseconds, for any of the resonant structures, resonant at frequencies between 1 and 7 MHz, that the bursts might excite, because the structures are all expected to have Q<5. So the resonant structures will respond to each burst independently, with no memory of the previous bursts, and it makes no difference if the train of bursts is coherent. In general, the biological response to a burst will depend only on the burst spectrum smoothed over a frequency range of about 20%. That is why the spectrum of FIG. 6 is characterized by its average value over bands covering component ranges of frequencies of a factor of 1.2, and not by the details of its structure at a finer scale. Physically, the δ-functions of spectrum 500 get smoothed out into the continuum of spectrum 402, by the low Q values of the resonant structures.

FIG. 6 shows a plot 600 of a burst spectrum 602 that barely satisfies the limitations of claim 1. Spectrum 602 is flat from 2.6 MHz to 4 MHz, and flat at a level that is lower by a factor of 2 from 4 MHz to 5.4 MHz, and is zero at other frequencies. effective spectrum 604, S_{eff}(W), was calculated in the range from 2 MHz to 6 MHz, as described above, from original spectrum 602, S(ω), as follows:

$${S}_{eff} ( ω ) = \frac{1}{2 π Q} \int d Δω S ( ω + Δ ω ) \frac{ω}{( {( Δω )}^{2} + {ω}^{2} / 4 {Q}^{2} )}$$

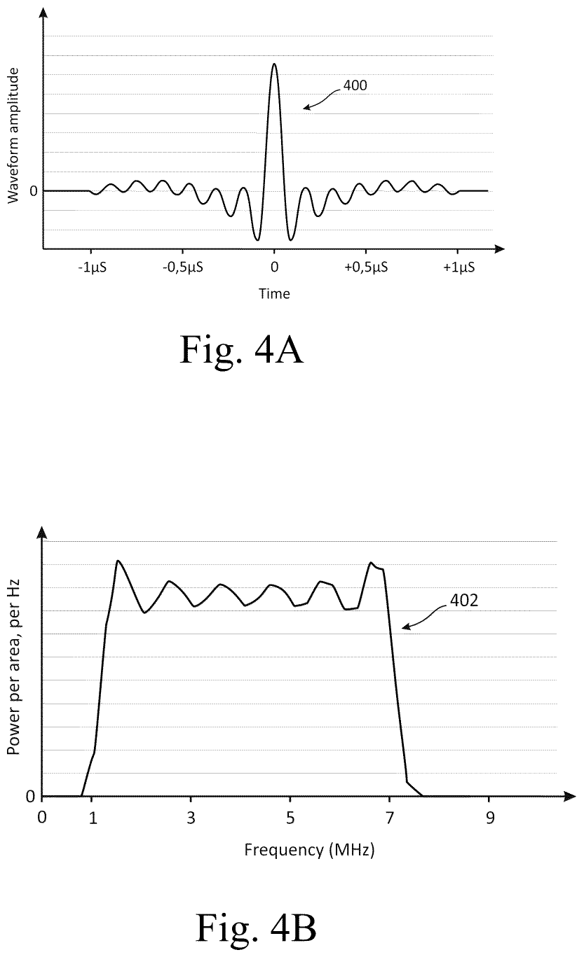

using Q=5. For this spectrum, 50% of the total power in the range from 2 to 6 MHz is found in 32% of the range, so spectrum 604 barely satisfies claim 1. Also for spectrum 604, 70% of the power in the range is found in the highest power portion that covers 50% of the range between 2 and 6 MHz, so by this alternative definition, spectrum 604 barely is “relatively uniform over a relatively broad range of frequencies.” The ratio of standard deviation to mean value for spectrum 604 is 46%, so by the definition that this ratio has to be less than 50%, spectrum 604 is also “relatively uniform over a relatively broad range of frequencies.” FIG. 7 shows a plot 700 of a spectrum 702 consisting of 2 narrowband peaks of equal height and width, at 3 MHz and at 5 MHz, that might have been used in prior art ultrasound therapy. Narrowband spectra of only 1, 2 or 3 frequencies are sometimes used in LIPUS therapy. The effective spectrum 704, S_{eff}ω), defined above in terms of the original spectrum 702, S(ω), is also shown in plot 700, using Q=5. If a narrowband component at 1 MHz were added to spectrum 702, it would have negligible effect on effective spectrum 704, in the range between 2 MHz and 6 MHz shown in FIG. 7. For spectrum 704, 50% of the total power in the range between 2 and 6 MHz is found in a highest power portion of the range that covers only 27% of the range. Also, 73% of the power is found in a highest power portion of the range that covers 50% of the range, so by the alternative definition, that requires less than 70% of the power to be found in that range, effective spectrum 604 also is not “relatively uniform over a relatively broad range of frequencies.” And the ratio of standard deviation to mean value of spectrum 604 is 64%, so spectrum 604 also is not “relatively uniform over a relatively broad range of frequencies,” by the definition that requires this ratio to be less than 50%.

These results, and the results described for FIG. 6, provide a sense of the criteria that can be used to distinguish spectra that are suitable for ultra-wideband ultrasound impact bursts, and spectra that are used in narrowband known ultrasound.

### Burst Waveforms Generated by Hardware

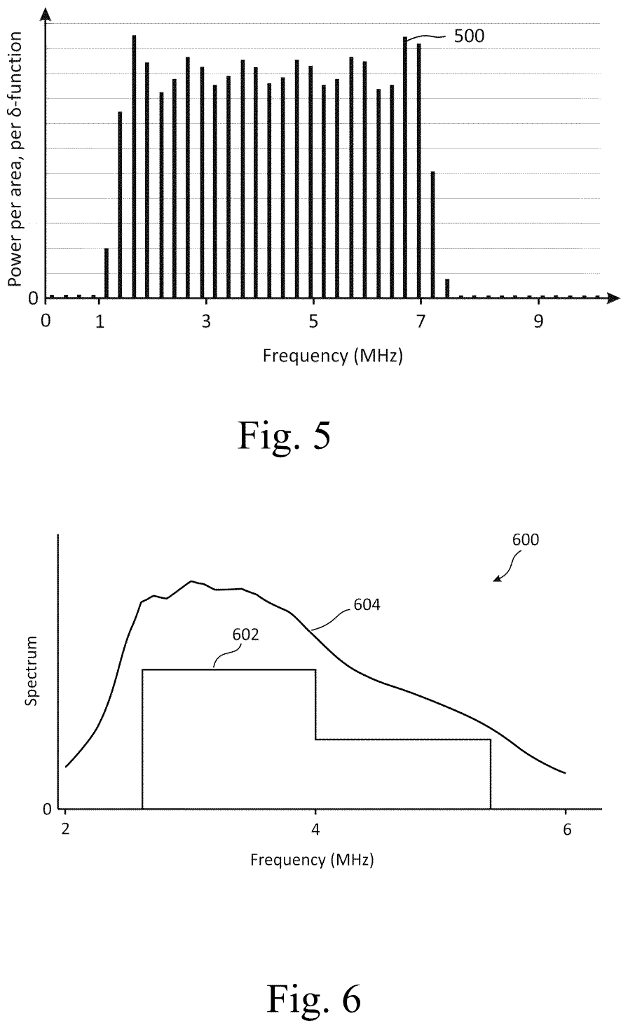

FIGS. 8A-8D illustrate an ultra-wideband ultrasound burst waveform and spectrum, similar to that shown in FIGS. 4A and 4 B, where the waveform signal can be generated by hardware, rather than synthesized digitally. Digitally synthesizing waveform signals may become impractical at frequencies of about 20 MHz and above, so it is useful to be able to generate waveform signals by hardware. Generating waveform signals at such high frequencies may start with the generation of bipolar square pulses, for example as described by Y. Onikienko et al, “High frequency Half-Bridge GaN-based pulse generator,” in 2019 IEEE 39th International Conference on Electronics and Nanotechnology (ELNANO), p. 700-703. The pulses are initially at least approximately square pulses, defined for example by their first few harmonics having amplitudes that are within 50% of the harmonic amplitudes that an ideal square wave would have. A train of such square pulses may be converted into a segment of a Sine wave by filtering out the higher harmonics. The filtered train of square pulses is at least approximately a sine wave, described for example in that their first few harmonics have amplitudes less than 50% of the amplitudes that an ideal square wave would have. This method can be used even to generate waveform signals at high power, and when energy efficiency is needed. At low power, a waveform signal that is a segment of a sine wave can be generated by using a sine wave generator, a selector, and a synchronizer.

FIG. 8A is a plot 800 of a set of 7 waveforms, labeled 802, 804, 806, 808, 810, 812, and 814, each consisting of a cosine function at a different frequency, all with the same constant amplitude for ω0 t between −π and +π, and zero amplitude everywhere else. The plots of the different waveforms are displaced vertically from each other so they can be clearly seen. The 7 frequencies used are ω₀, 2ω₀, 3ω₀, 4ω₀, 5ω₀, 6ω₀, and 7ω₀. If ω₀=2π×10⁶ radians per second, then the frequencies are 1 MHz, 2 MHz, 3 MHz, 4 MHz, 5 MHz, 6 MHz, and 7 MHz, and each waveform lasts for 1 microsecond. The 7 waveforms respectively have 1, 2, 3, 4, 5, 6, and 7 full wave periods in that microsecond.

FIG. 8 B shows a plot 816 of the sum S (t) of all the waveforms shown in FIG. 8A,

$$S ( t ) = {\sum }_{k = 1}^{7} cos ( k {ω}_{0} t ) for \vert {ω}_{0} t \vert < π$$

$$S ( t ) = 0 otherwise$$

The sum looks very similar to waveform 400 in FIG. 4A. The reason for this is apparent in FIG. 8C, which shows overlapping plots of the Fourier transforms 818, 820, 822, 824, 826, 828, and 830 of the seven waveforms shown in FIG. 8A. The plots have been displaced vertically from each other so they can be clearly seen. Each Fourier transform consists of two sinc functions centered at the positive and negative frequencies of the cosine function used to generate that waveform. Each sinc function has a central peak that extends a distance ω₀ to its first zero on either side. Because the first zero of each sinc function corresponds to the peak of the neighboring sinc function in each direction, and because the sinc function is much smaller in magnitude outside its central peak, the sum of all the sinc functions is roughly constant between +ω₀ and +7ω₀ and between −7ω₀ and −ω₀, and much smaller in magnitude everywhere else. In other words, the sum of all the sinc functions in FIG. 8C looks similar to the Fourier transform of waveform 400 in FIG. 4A, so it is not surprising that waveform 816 in FIG. 8B, which is the Fourier transform of the sum of all the sinc functions in FIG. 8C, would look similar to waveform 400 in FIG. 4A.

FIG. 8D shows a plot 832 of the spectrum of waveform 816 in FIG. 8B. The spectrum is a reasonably good approximation to spectrum 402 of FIG. 4B, being roughly constant between +ω₀ and +7ω₀, and much smaller in magnitude everywhere else, but with somewhat larger wiggles and distortions than spectrum 402 in FIG. 4B. If, as the inventors believe, spectrum 402 in FIG. 4B is a useful spectrum for a therapeutic ultrasound burst to have, then we expect that spectrum 832 in FIG. 8D, which has the potential advantage that the burst can be generated by hardware, will also produce good results in ultrasound therapy.

It should be noted that even though 7 different signal components were used to produce waveform 800 or FIG. 8A, a good approximation to this waveform could probably be produced using somewhat fewer signal components that are generated by hardware, for example 4, 5 or 6 components. In particular, the higher frequency components could probably be spaced further apart in frequency, as long as adjacent frequency components are not more than 20% different in frequency, and the duration of each component could be made somewhat shorter than 1 microsecond, so that the sinc functions for the different components would still overlap in frequency.

### Test Results

The inventors and their colleagues performed a number of tests showing the therapeutic effectiveness of ultra-wideband ultrasound bursts. Some of those tests and their results will be described here. For the experiments, multicomponent dual-band ultra-wideband wave packets were generated, as described in detail in the text of the invention. The low-frequency range of 1-3 MHz represents a Sinc signal from which frequencies up to 1 MHz were subtracted using bandpass filtering. In the high-frequency range, the following frequency bands were used: 3-7; 3-9; 3-14; and 3-19 MHz, which represent Chirp signals. Irregularities (ripples) in the frequency characteristics were suppressed by window filtering.

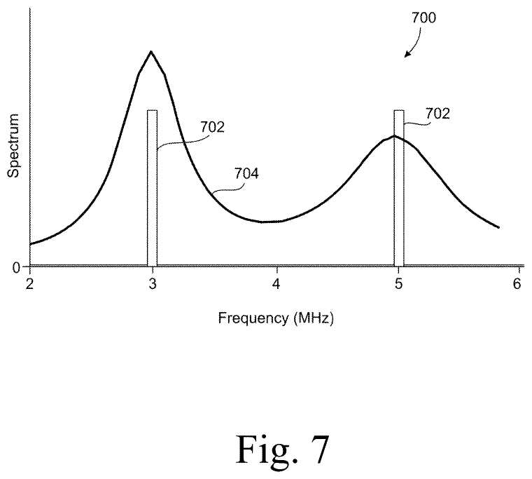

Both signals, the low-frequency and high-frequency, were simultaneously summed in a spectrum corrector and converted into ultrasound. The generated signals were emitted to the target location of the object as a wave packet with a constant frequency power density in frequency bands of 1-6; 1-8; 1-14; and 1-19 MHz. An example of such a signal is shown in FIG. 9A.

FIG. 9A illustrates a biophysical experiment on the dependence of fibroblast proliferation on the frequency band of the multi-component-dual-band signal were studied. The following signals were used in the experiment (see Table): a multicomponent signal Sinc signal and a high-frequency ultra-wideband part of the signal, in which the main part of the energy is concentrated. The total duration of the high-frequency and multicomponent signals differed many times (see Table). In the table and FIG. 9B, UMI Burst is Ultra-wideband Impact Burst.

### Materials and Research Methods

Cell lines 3T3-a31 (mouse fibroblasts) were obtained from the cell bank of lines from human and animal tissues of the Kavetsky Institute of the National Academy of Sciences of Ukraine. RPMI-1540 medium (L0496-500. Biowest France, DMEM (L0102-500, Biowest France).

Serum. Fetal Calf Serum (S181B-500, Biowest France).

Solutions and dyes. Phosphate buffered saline (PBS) (L0615-500 Biowest France); 0.02% Na-EDTA solution (Versen) (batch No. 6, Vetline Agrostience, Ukraine; ethanol (Pharma-Net); 0.25% trypsin solution (L0910-100, Biowest France); trypan blue (A0668 Applichem, Germany), 0.4% crystal violet solution (4058311000, ThermoScientific, USA).

Other materials. Plastic cell culture vessel (SPL, Korea), sterile test tubes (Falcon, USA), sampler tips with a volume of up to 1000 μl (BSR002/05261, Eppendorf, USA)/

Devices. Ultrasonic treatment unit. Described in the text as an example of the invention. Class II biological safety cabinet BL2-900 (Kupertek Breva, Ukraine), CO2 incubator (Heal Force, China), inverted microscope Axiovert 25 (Carl Zeiss, Germany), microscope Axiostar Plus (Carl Zeiss, Germany), Goryaev chamber (Voles, Ukraine), mini-shaker PSU-2T (BioSan, Latvia), multi-well spectrophotometer (Labsystems Multiskan PLUS, Finland), laboratory centrifuge (Biosan LMC-3000, Latvia), automatic pipettes with a volume of up to 20 μl (Eppedopf AG, Germany), 200 μl (Ratiolab, Germany) and 1000 μl (Ratiolab, Germany), refrigerator (Snaige, Lithuania).

Cultivation of cell lines in vitro. The cells were cultured in RPMI-11640/DMEM medium with 10% fetal calf serum, 1× penicillin-streptomycin in a humidified atmosphere with 5% CO2 at 37° C.

After the cells formed a dense monolayer on the substrate (2-3 days of growth), they were sifted, and the cells were dissociated using PBS with 1 mM EDTA (Versene solution) and trypsin [R. J. Freshney, Culture of Animal Cells: A Manual of Basic Technique and Specialized Applications, Sixth Edition, 6th Ed. John Wiley & Sons. Inc., Wiley-Blackwell, 2010.]. Cells were counted using a Goryaev chamber using the vital dye trypan blue. The number of cells was determined by the formula: A/80×2=X·10⁶ cells per milliliter of medium, where A is the number of cells that were counted in the Goryaev chamber.

Indexes of proliferation of normal 3T3-A31 mouse fibroblasts after 5 sessions of sonication are shown in Table and FIG. 9B.

| Signal | Control | UMI Burst Low intensity | | | | | UMI Burst Middle intensity | | | |
|---|---|---|---|---|---|---|---|---|---|---|
| Intensity | 0 | 1 μW/cm² | | | | | 1 mW/cm² | | | |
| Bandwidth, | 0 | 2 | 8 | 14 | 19 | 0 | 2 | 8 | 14 | 19 |
| MHz | | | | | | | | | | |
| Proliferative | 0 | 2.9 | 4.4 | 36.7 | 13.2 | 1.5 | 0.9 | 20.6 | 36.7 | 4.4 |
| Index PI | | | | | | | | | | |

The experiments were carried out in the following frequency ranges 2 MHz, 8 MHz, 14 MHz, and 19 MHz. 0 MHz refers to a control sample without UMI Burst impact.

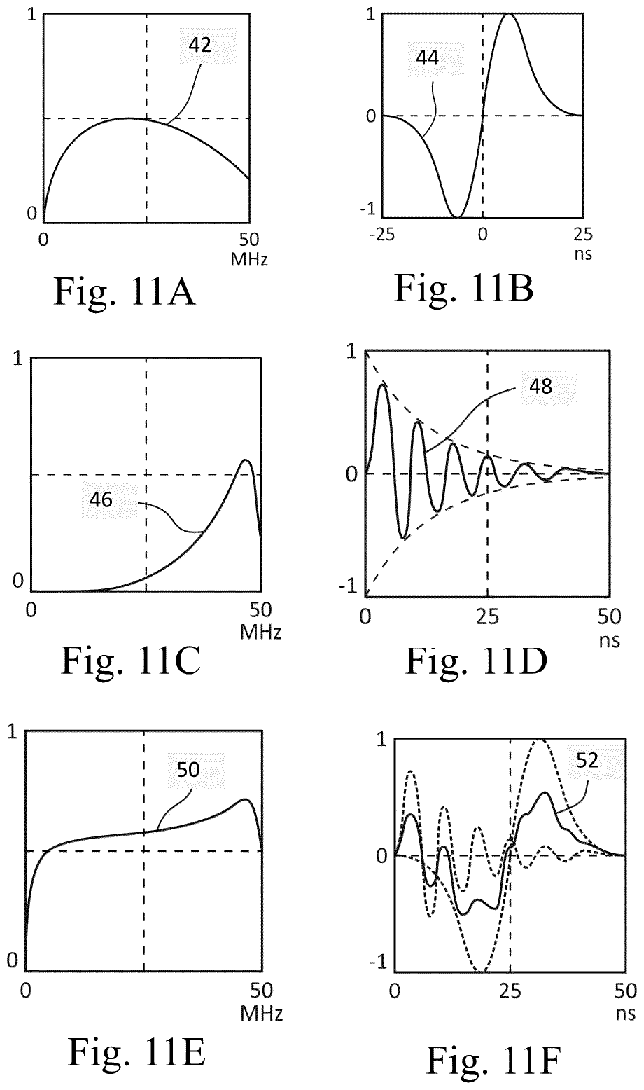

The biological effect characterized by the proliferative index for all bands of signals is supposed to be the same. Table and FIG. 15, however, shows that the wider bandwidth of the signals results in higher proliferative and a more pronounced biological effect. For example, with a frequency band of 2 MHz, the proliferation index is 2.9, and with a frequency band of 14 MHz, it is 36.7. in other words, the maximal experimentally obtained difference between narrow and wide signals is about 12.7 times.

FIG. 9C shows the results of a study of wound-healing in mice, comparing a control group of mice with mice treated with LIPUS ultrasound, and mice treated with ultra-wideband ultrasound bursts. The study was done with a sample of 40 female BALB line mice. Standardized linear wounds were inflicted on their backs, 17 mm long and 1.2 mm deep. The control group of mice was not treated at all. The group treated with LIPUS were treated with 30 mW/cm² (at the peak intensity location along the length of the beam, averaged over the cross-section of the beam, and averaged over time during the duration of the treatment session) of 1.5 MHz ultrasound, in 200 μs long pulses with a 1 kHz repetition rate, for a duration of 5 minutes each day, for 9 days. The group treated with ultra-wideband ultrasound bursts were treated with 1 μW/cm² per MHz bursts with frequencies between 1 and 7 MHz (at the peak intensity location along the length of the beam, averaged over the beam cross-section, average over the duration of the treatment session, and averaged over the 1-7 MHz frequency range), with a 1 kHz burst repetition rate, for a duration of 5 minutes each day, for 9 days. The total power per area, integrated over the 1 to 7 MHz frequency range, would be 6 μW/cm².

A SigLent SDG 6052X DDS generator from SigLent Technologies Co. was used to generate the ultrasound. The head for emitting LIPUS and ultra-wideband bursts contained a multimode ultra-wideband transducer with a diameter of 20 mm. LIPUS and ultra-wideband bursts were delivered to the affected area on the back of the mouse through a layer of ultrasonic gel UBQ 5000 Ultragel.

FIG. 9 shows a plot 900 of the average length of the wound on each day of the study, for mice in each group. The vertical axis shows the average length of the wound in mm, and the horizontal axis shows the number of days. Curve 902 shows the results for the control group, curve 904 shows the results for the group receiving LIPUS treatment, and curve 906 shows the results for the group receiving treatment with ultra-wideband bursts. In the control group, inflammation and edema were observed in the wound area for the first two days, and thereafter the average wound length decreased by at most 2.1 mm per day. In the group receiving LIPUS, inflammation and edema were absent, but the average rate of healing thereafter was about the same as for the control group, a maximum of 2.1 mm per day. For the group receiving ultra-wideband bursts, inflammation and edema were also absent, and the healing rate after the first 2 days was faster, with the average wound length decreasing by up to 3.8 mm per day. By 9 days, the wounds of all the mice in that group were fully healed, while the average wound length in the control group was 4 mm, and the average wound length in the LIPUS group was 3 mm.

Another study showed the effects of ultra-wideband bursts at two different intensity levels, and LIPUS, on the blast transformation, in vitro, of T-lymphocytes, a process that activates the body's immune response. The blast transformation was greatly enhanced by ultra-wideband bursts, especially at the lower power level of 1 μW/cm² per MHz. This lends support to the idea that ultra-wideband ultrasound bursts might be therapeutically useful.

As in the study of wound-healing in mice, a SigLent SDG 6052X DDS generator was used, with a head containing a multimode ultra-wideband transducer with a diameter of 20 mm, for transmitting the LIPUS and the ultra-wideband ultrasound bursts. The LIPUS was at 1.5 MHz, at 30 mW/cm², at the peak intensity location along the length of the beam, averaged over the beam cross-section, averaged over the beam cross-section and time averaged over the duration of the treatment session. The ultra-wideband bursts were used both at 30 mW/cm² per MHz and at 1 μW/cm² per MHz, with the bursts having frequencies between 1 and 7 MHz, at the peak intensity location along the length of the beam, averaged over the beam cross-section, time averaged over the duration of the treatment session, and averaged over the 1 to 7 MHz frequency range.

10 ml of blood from a subject's vein was taken into a test tube with heparin (25 U/ml). The contents of the test tube were mixed carefully and left for 60 minutes in a thermostat at 37° C. to precipitate red blood cells. After incubation in a thermostat, the supernatant plasma layer, which was enriched in leukocytes, including T-lymphocytes, was aspirated into a separate sterile test tube and the number of leukocytes in 1 ml was determined. Then the suspension of leukocytes with nutrient medium 199 (containing 200 IU of penicillin and 100 IU of streptomycin per ml) was diluted so that 1 ml contained 1 to 2 million leukocytes, in a medium that was 20% autologous plasma and 80% nutrient medium. The prepared leukocyte suspension was poured into sterile 1 ml vials and a gas mixture containing 5% carbon dioxide was passed through it to maintain sterility. The vials were placed in a thermostat at 37° C. for 5 days. During each of the 5 days, the samples were exposed for 5 minutes to the LIPUS ultrasound, or to the ultra-wideband bursts at one of the two power levels, or in the case of the control samples were not exposed to ultrasound at all. At the end of the 5 days, the samples were examined in an optical microscope, and it was determined what percentage of T-lymphocytes had undergone blast transformation and were visibly different from normal cells.

For the control sample, 7.4% of the cells had transformed. For the sample exposed to LIPUS, 6.9% of the cells had transformed. For the sample exposed to UWB bursts at 30 mW/cm², 10.6% of the cells had transformed. For the sample exposed to UWB at 1 μW/cm². 23.5% of the cells had transformed.

Another study looked at the effects of ultra-wideband ultrasound bursts on the proliferation of hematopoietic progenitor cells (HPCs) and hematopoietic stem cells (HSCs) in the bone marrow of mice. A strong positive effect was found, which may be evidence for the usefulness of ultra-wideband bursts for promoting regenerative processes in which stem cells play a role.

Ultra-wideband ultrasound bursts were used with an intensity of 1 μW/cm² per MHz, at the peak intensity location along the length of the beam, averaged over the cross-section of the beam, averaged over the duration of the treatment session, and averaged over frequency, with frequencies in a range between 1 and 20 MHz, and a pulse repetition rate of 100 kHz. The duration of the exposure in each session was 5 minutes, and the number of sessions was 10. The study was conducted on 45 sexually mature (3 months) female mice of the BALB/c line, with 15 animals each in the control and experimental groups. All work with experimental animals was carried out in compliance with the “European Convention for the Protection of Vertebrate Animals Used for Experimental and Other Scientific Purposes”, as well as the principles of bioethics and biological safety standards.

After euthanasia of the animals, the bone marrow content was separated, the resulting suspension was resuspended, and nucleated cells were counted in a Neubauer hemocytometer. Phenotyping of the cell suspension by CD31, CD34, and CD45 markers was performed using fluorochrome-labeled rat monoclonal antibodies to mouse membrane antigens according to the manufacturer's recommendations (BD Pharmingen, USA). Measurements were performed on a BD FACSAria laser flow cytometer-sorter (Becton Dickinson, USA) using BD FACS Diva 6.1.2 software. To adjust the compensation of overlapping emission spectra of fluorochromes in multiparameter analysis, control samples of cells without introduction of antibodies (unstaining control), samples with each of the antibodies separately (single staining control) and samples with a combination of several antibodies without one (fluorescence minus one-FMO control) were used.

According to the gating protocol of the International Society for Hemotherapy and Tissue Engineering (ISHAGE), among all nucleated cells (total nucleated cells, TNCs), the relative content of haematopoietic progenitor cells (HPCs) with the CD34+45dim/−/phenotype was determined, as well as hematopoietic stem cells (HSCs) with the CD34+45dim/−/phenotype, which morphologically correspond to mononuclear cells. Statistical analysis was performed using the non-parametric Mann-Whitney U-test. Data are presented as mean and standard deviation (Mean±SD). Differences between groups were considered significant at p≤0.05.

To exclude possible changes in the absolute content of nucleated cells in the bone marrow in the femur due to the effect of the UMB Burst factor, the absolute number of cells without and as a result of the effect was previously compared, and it was established that there were no significant differences.

The results of immunophenotyping of subpopulations of hematopoietic and endothelial progenitor cells in the bone marrow of mice of the studied groups are shown in Table 1.

TABLE 1
Relative content of subpopulations of HSCs and HPCs inthe
bone marrow of mice of studied groups, in percent

| Group | |
|---|---|
| 1 (control) | 2 (experimental) |

| Cell subpopulations | HSCs | HPCs | HSCs | HPCs |
|---|---|---|---|---|
| Indicators of all | 0.049 | 1.6 | 0.058 | 4.6 |
| experimental animals | 0.047 | 2.1 | 0.069 | 3.2 |
| | 0.229 | 4.2 | 0.166 | 3.4 |
| | 0.039 | 2.7 | 0.305 | 6.7 |
| | 0.213 | 3.7 | 0.192 | 4.0 |
| | 0.122 | 2.8 | 0.367 | 8.2 |
| | 0.327 | 4.8 | 0.332 | 4.5 |
| | 0.099 | 3.6 | 0.430 | 6.7 |
| | 0.132 | 5.3 | 0.472 | 12.6 |
| | 0.231 | 7.0 | 0.432 | 7.5 |
| | 0.071 | 1.2 | 0.276 | 20.9 |
| | 0.077 | 2.7 | 0.519 | 6.3 |
| | 0.245 | 3.7 | 0.208 | 4.2 |
| | 0.228 | 3.8 | 0.430 | 8.2 |
| | 0.316 | 7.7 | 0.586 | 14.7 |
| Mean | 0.162 | 3.8 | 0.323 | 7.7 |
| SD | 0.099 | 1.8 | 0.160 | 4.9 |

In the experimental group, the relative content of hematopoietic stem (p=0.008) and hematopoietic stem progenitor (p=0.004) cells in the bone marrow was significantly higher than in the control group. Given the absence of an increase in the absolute content of nucleated cells under the influence of the ultra-wideband bursts, this may indicate the stimulation of the proliferation of early stem cells or the reprogramming and transformation of a portion of the somatic cells into stem cells.

Another study performed by the inventors looked at the effect of ultra-wideband ultrasound bursts, and the effect of narrowband ultrasound waves at 1 MHz, 3 MHz, or 7 MHz, on the proliferation of mouse fibroblasts in vitro. The results showed a net increase in proliferation over apoptosis caused by the ultra-wideband bursts. Since fibroblasts support the formation of connective tissue, this result supports the idea that ultra-wideband ultrasound bursts may be therapeutically useful for promoting wound-healing, for example.

The study was performed on normal fibroblasts of BALB/c mice, of cell line 3T3-A31. The same SigLent SDG 6052X ultrasound generator, with the same ultra-wideband multimode transducer head, as described above, was used for this study. The fibroblasts, in a nutrient medium, were exposed to ultra-wideband ultrasound bursts with a spectrum having frequencies between 1 and 7 MHz, at an intensity of 30 mW/cm² per MHz, at the peak intensity location along the length of the beam, averaged over the cross-section of the beam, time averaged over the duration of the treatment session, and averaged over the 1 to 7 MHz frequency range; or to narrowband ultrasound waves at 1 MHz, 3 MHz, or 7 MHz, at an intensity of 30 mW/cm², at the peak intensity location along the length of the beam, averaged of the cross-section of the beam, and time averaged over the duration of the treatment session. For both the ultra-wideband bursts and the narrowband ultrasound, the duration of each treatment session was 5 minutes, with one treatment session performed per day for 5 consecutive days. A control sample of fibroblasts was not exposed to ultrasound at all. 24 hours after the last treatment session, the fibroblasts, dyed with Trypan blue dye from Hy Clone in the USA, were observed and photographed under an Aviostar Plus binocular microscope from Carl Zeiss of Germany. A digital camera, the Goryaeva camera from Farmmedteh of Ukraine, was used to photograph the fibroblasts, and to count the number of live and dead cells in the field of view of the microscope. The number of live cells counted after each of the treatment methods was expressed as a percentage of the number of live cells counted in the control group sample, and error bars due to the finite numbers of cells were determined.

In the sample that was exposed to narrowband 1 MHz ultrasound, the number of fibroblasts counted was 62%±5% of the number in the control group. This indicates that narrowband 1 MHz ultrasound at this intensity causes more apoptosis than proliferation of fibroblasts to occur, which is consistent with the results reported for a different type of cells by Mingfang Shi et al, “Low intensity-pulsed ultrasound induced apoptosis of human hepatocellular carcinoma cells in vitro,” Ultrasonics 64 (January 2016), 43-53, available at https:(slash)(slash)doi(dot)org(slash)10.1016(slash)jultras.2015.07.011.

In the sample that was exposed to narrowband 3 MHz ultrasound, the number of fibroblasts counted was 96%±13% of the number in the control group, which was not a statistically significant difference, indicating a balance between any differences in apoptosis and proliferation of fibroblasts caused by the ultrasound.

In the sample that was exposed to 1-7 MHz ultrasound, the number of fibroblasts counted was 120%±14% of the number in the control group.

In the sample that was exposed to the ultra-wideband bursts, the number of fibroblasts counted was 138%±15% of the number in the control group, showing a clear net increase in proliferation over apoptosis caused by the ultra-wideband bursts at this intensity.

### Section 1

Section 1, and Section 2 below, provide some examples of structures and methods, some definitions that may also be used in some embodiments of the invention described elsewhere, and some references that are cited is Section 1 and Section 2. The numbering of the references starts over again in Section 2.

The present invention relates to micromechanical methods of impact, in particular to non-invasive ultra-wideband micromechanical impact burst used in research, technological, biological, medical and cosmetic equipment to affect diseased, damaged or altered, including aged or infected, biological tissues and somatic cells in order to enhance and speed up the processes their regeneration and recovery. In some embodiments of the invention, the method comprises applying ultra-wideband micromechanical impact bursts (UMI Bursts) to the tissue for acceleration of reparative and regenerative processes to multiple levels of cellular and tissue structures hierarchy of the tissues, for example having dimensions from about 10-4 mm to about 1.5 mm.

Some embodiments of the invention include activation and transformation of specialized somatic cells into pluripotent and progenitor stem cells. The micromechanical energy of UMI Bursts non-invasively transferred to a great amount of mechanosensitive elements of pathologically altered and adjacent healthy tissues in the form of UMI Bursts is optionally formed by at least two signals-main and correcting. The frequency spectra of UMI Bursts contain a very large number of frequencies in one or several ultra-wideband ranges, and which have a broadband rate of 0.2<η≤0.992, which corresponds to the frequency range 1.0-250.0 MHz.

### Ultra-Wideband Micromechanical Method for Modulation of Cellular Activity

Some embodiments of the present invention relate to ultra-wideband micromechanical methods of impact, in particular to non-invasive ultra-wideband micromechanical bursts used in research, technological, biological, medical and cosmetic equipment to affect diseased, damaged or altered, including aged or infected, somatic cells and biological tissues in order to enhance and speed up the processes their regeneration and recovery.

### Definitions

For the purpose of this invention, the following terms employed herein and in the appended claims refer to the following concepts:

“Micro-energy modulation impact”—the impact in the treatment area of ultra-wideband micro-energy micromechanical signals with a maximum peak energy of 0.03-0.1 mJ/mm², and a spatial-peak temporal average intensity of less than hundreds of microwatts per square millimeter [1].

“Induced stem cells”—undifferentiated pluripotent or progenitor stem cells obtained from somatic cells by their reprogramming;

“Regenerative impact”—restoration of diseased or damaged tissues and organs of a biological object using pluripotent and progenitor stem cells, activated, transplanted or transformed [2];

“Regenerative cosmetology”—a technology for rejuvenating (revitalizing) aging skin by activation or transplantation of stem cells, or induced transformation of somatic and progenitor cells into stem cells [3];

“Ultra-wideband (UWB) system and signals”—the fractional bandwidth n and the frequency band ratio br of signals which correspond to the following values:

$$η = 2 \frac{{f}_{h} - {f}_{l}}{{f}_{h} + {f}_{l}} + 2 \frac{{b}_{r} - 1}{{b}_{r} + 1} \geq 0 , 2 ,$$

where fₕ and fₗ are the upper and lower frequencies of the spectrum of signal at the level of 10 dB, bᵣ is the ratio:

$${b}_{r} = \frac{{f}_{h}}{{f}_{l}} = \frac{2 + η}{2 - η} \geq 1 , 22.$$

In accordance with the Standards [4-10] and scientific research [11-12]:

| | |
|---|---|
| Fractional Bandwidth η | Band Ratio bᵣ |

Narrowband

| | |
|---|---|
| 0.00 < η ≤ 0.01; | 1.00 < bᵣ ≤ 1.01, |

Wideband

| | |
|---|---|
| 0.01 < η < 0.2 | 1.01 < bᵣ ≤ 1.22 |

Ultra-wideband

| | |
|---|---|
| 0.2 < η < 2.00 | 1.22 < bᵣ < ∞ |

“Ultra-wideband Micromechanical Impact Burst (UMI Burst)”—propagating in medium detached ultra-wideband micro-mechanical disturbance of the medium, which differs in frequency and spatial characteristics from known narrowband and wideband ultrasound;

“Ispta—narrowband signal intensity”—spatial-peak temporal-average intensity of narrowband signal or the sum of narrowband signals, averaged over the cross section of the ultrasonic beam, impulse rate and over time.

“Isptaf intensity of wideband and ultra-wideband signals intensity, in particular, ultra-wideband micro-mechanical impact bursts (UMI Bursts)”—the intensity averaged over cross-section of the burst radiating beam, as well as over time, impulse rate and over burst spectrum.

“Vibration modes of an electromechanical transducer”—a set of natural or forced vibrations of a transducer with different physical characteristics, for example: resonance modes—radial R, edge E, angular A, volume V, as well as planar ultra-wideband P-mode—vibration of the surface layer of the transducer;

### REFERENCES CITED IN THE ABOVE DEFINITIONS

1. Yegang Chen et al. Role and Mechanism of Micro-energy Treatment in Regenerative Medicine. Translational Andrology and Urology. Feb. 8, 2020 doi org/10.21037/tau.2020.02.25.
2. A. Atala (editor) et al. Principles of Regenerative Medicine, Academic Press, 2019, 1416 pages.
3. Sucharita Boddu et al. Regenerative Medicine in Cosmetic Dermatology. Review. Cutis. 2018 January; 101 (1): 33-36.
4. OSD/DARPA, Ultra-Wideband Radar Review Panel,” Arlington, VA, Defense Advanced Research Project Agency (DARPA), 1990.
5. US Federal Communication Commission (FCC), Part 15, October 2003, http://www(dot)fcc(dot)gov/oct/info/rules.
6. EC 2009 Commission of the European communities Decision 2007/131/EC April 2009.
7. International Electrotechnical Commission (IEC), Basic EMC Publication 6100-2-13: “Environment-High-power Electromagnetic (HPEM) Environment-Radiated Conducted”. American National Standards Institute (ANSI) ANSIC63.14-1998, American National Standards
8. Dictionary for Technologies of Electromagnetic Compatibility (EMC), Electromagnetic Pulse (EMP), and Electromagnetic Discharge (ESD), October 1998.
9. Manual of Regulations and Procedures for Federal Ratio Frequency Management National Telecommunication and Information Administration. Report of May 2003 (rev. September 2004).
10. Institute of Electrical and Electronics Engineers (IEEE), IEEE Std 6861997, IEEE Standard Radar Definitions, 16 Sep. 1997.
11. Oyan, M. J. et al. “Ultrasound Gates Step Frequency Ground-Penetrating Radar”. Geoscience and Remote Sensing, IEEE Transactions, vol. 50, No. 1, pp. 212-22, January 2012
12. F. Sabath et al. “Definition and Classification of Ultra-Wideband Signals and Devices”. Radio Science Bulletin (2005), No 313 pp. 12-20.

In regenerative impact, the restoration of degraded, diseased or damaged biological tissues is carried out by transplantation of exogenous stem cells (SC) and activation of endogenous SC [13].

Stem cells originate from two main “sources”: tissues of an adult organism and embryos. Scientists are also working on ways to obtain stem cells from other cells using reprogramming techniques.

The sources of exogenous SC for transplantation are embryos, umbilical cord blood, bone marrow and adipose tissue. SC transplantation is widely used, despite the immunogenicity and oncogenicity of exogenous SC [14].

SCs are found in many tissues of the body, including the brain, bone marrow, blood and blood vessels, skeletal muscles, skin, and liver. It has been established that such and similar resident endogenous SCs, clue to the home effect, can be accumulated in the treatment area and accelerate the regeneration process [15].

In regenerative impact-treatment, autologous extracorporeal pluripotent SCs are often used, isolated, for example, from the adipose tissue of the body. They are propagated in an artificial nutrient medium and returned to the body. Disadvantages of the technology for extracorporeal cultivation of autologous SCs are their high cost, incomplete compatibility with resident cells, carcinogenicity arising from accumulated differences in the process of cell reproduction, and the presence of biochemical growth factors necessary for the growing process. It is safest to use your own non-multiplied SCs. They are obtained either from the bones in the body marrow or with the help of drugs that displace SC into the blood. Using this technology, it is possible to obtain only a small amount of SC [16].

In 2006-2012, Yamanaki Shinya and co-author showed [17-18] (John B. Gordon, Shinya Yamanaka. The Nobel Prize in Physiology or Medicine 2012) that in principle, an induced transformation (reprogramming) of any adult somatic cell into a pluripotent young stem cell is possible, and, in the future, its transformation into any specialized cell. Reprogramming took place in the cell culture due to the “cocktail” of Yamanaki—a set of four modified genes implanted into the genome of somatic cell generations of proliferating cells. The long-term consequences of such a transformation for the organism are still insufficiently understood. In addition, the extracorporeal production of modified cells using the Yamanaka technology is still very expensive.

Later it was found that somatic cells can be reprogrammed in SC not only biochemically, but also physically. Strong, on the verge of cell death, compression or stretching of adult cells leads to their massive transformation into SC [19]. Moreover, the impact of even weak mechanical signals acting on the extracellular matrix also leads to reprogramming, i.e., transformation of a part of cells into SC [20-21].

Weak mechanical signals can trigger many chains of informational and biochemical transformations like an avalanche [22].

A new scientific discipline called “mechanobiology” has appeared, aimed at studying the control of the properties of cells and tissues under the influence of mechanical forces. Specialists in mechanobiology have established [23-27]:
generation and transmission of mechanical signals occurs at all levels of the cell structures. The cell nucleus, including DNA and chromosomes, are sensitive mechanosensors. Mechanical informational signals come from the environment of cells into the intracellular cytoskeleton and along the filaments further into the nucleus. Deformation and conformal transformations of intranuclear structures are triggered, and changes in gene regulation and configuration of epigens are initiated. Mechanical signals in cells control numerous processes, including transformation of the epigenome and migration of methylating markers. Cells also exchange mechanical signals when their membranes are in contact [28].

It is obvious that external mechanical signals, similar to the signals of the cells themselves, can effectively influence the processes of their vital activity and transformation. Scientists became interested in the possibility of reprogramming cells by excitation a special type of mechanical vibration—ultrasound [29].

The advantage of ultrasound is the non-invasive introduction of therapeutic signals into the body, therefore, changes in cells and tissues occur in their natural environment. There are no chemical factors for reprogramming, which dramatically reduces the risk of developing tumors and immune rejection of “foreign” cells.

The new type of ultrasound is needed, in which the mechanobiological effect prevails and are excluded the thermal, acoustochemical, acoustoelectric effects inherent in conventional impact-therapeutic ultrasound.

This type of ultrasound was proposed by Duarte L. R. back in 1985 (U.S. Pat. No. 4,530,360 [30]). To date, in low-intensity ultrasound treatment, narrow-band amplitude-modulated harmonic ultrasound is mainly used with an intensity of less than 200.0 mW/cm², most often 1.0-30.0 mW/cm², with a frequency about of 1.5 MHz. The duration of each ultrasonic pulse is 200 μs, the duty cycle is 0.2. Impact-treatment with such impacting signals came to be called Low Intensity Pulsed Ultrasound Impact (LIPUS).

Broadband rate η of LIPUS signals is η<0.2.

In 1999, Duarte L. R. (U.S. Pat. No. 5,904,659 [31]) significantly expanded the boundaries of the operating frequencies of signals. The carrier frequency is proposed in the range of 20.0 kHz-10.0 MHz, the frequency of the modulating signal is from 5.0 Hz to 10.0 kHz, the Ispta intensity (spatial peak temporal average acoustic intensity) is less than W/cm² (100 mW/cm²). Broadband rate η is η<0.2.

Already the first clinical applications of LIPUS have shown its effectiveness in the treatment of bone fractures, especially poorly healing ones, as well as in accelerating the healing of soft tissue injuries.

It has been clinically shown that LIPUS is a non-invasive method for stimulating tissue and cell bioactivity.

In US20060241522A1 Chandraratna H. describes a method of therapeutic treatment for ischemia and other cardiovascular disorders. The application of low-intensity ultrasound with frequencies in the range of 40.0 KHz-8.0 MHz, mainly 1.6-4.0 MHz, with an intensity not higher than the intensity of diagnostic ultrasound is claimed.

The use of LIPUS in dentistry has proven to be effective. Bone tissue regeneration, treatment of periodontal disease, and acceleration of implantation are clinically indicated [34].

Several years ago, the possibility of LIPUS treatment of skin tumors was shown, in particular, basal cell carcinoma, squamous cell carcinoma and melanoma (WO 2015077006, Boer Miriam Sara et al. [35]). Ultrasound was used with frequencies of 27.0 kHz and 2.2 MHz, and intensity in the focal zone of 0.17 W/cm² and 5.0 W/cm², i.e., LIPUS was combined with low-frequency ultrasound.

In the invention US20160067526 there is proposed the use of LIPUS for the treatment and/or prevention of neurodegenerative diseases. A focusing ultrasonic transducer with an operating frequency of 1.0 MHz, which emits pulses with a duration of 50 ms and a duty cycle of 0.05 was used. The intensity of Ispta ultrasound at the surface of the transducer was 110 mW/cm², and at the focus 528 mW/cm². It is stated that ultrasound can be with frequencies from 20.0 kHz to 16.0 MHz, and intensities from 1.0 mW/cm² to 1.0 W/cm².

In US20060241522A1, Chandraratna H. [37], it was shown that LIPUS can also be used for the regeneration of peripheral nerves.

Shields in US20070249938 μsed LIPUS two-frequency sequences for living tissue treatment.

A modification of LIPUS with an intensity increased to 400 mW/cm² was proposed by Min et al. for the treatment of edema (US20100204618 A1 [39]).

Huckle J. et al in 2010 patented the use of LIPUS for the treatment of connective tissue pathologies, and hence diseases of the musculoskeletal system [40].

In 2010, Schwartz D. in patent US2010152626 A1 proposes ultrasound treatment of glaucoma [42].

Zhou J. et al in 2018 showed that LIPUS protects retinal ganglion cells and reduces the consequences of their injury [42].

In 2013, EP Global Communications Inc. (USA) announced advanced technology and device for macular degeneration and retinitis pigmentosa by emitting low intensity ultrasound into the eye for the purpose of regeneration of damaged cells and to possibly stopping the degeneration of existing healthy cells within the macula and the entire retina [43].

In 2018 it was found by Zhou L. X. et al. [44], that low-intensity pulsed ultrasound protects retinal ganglion cells from damage to the optic nerve, which can stop the development of glaucoma.

The list of the above-mentioned diseases and pathological stages for which LIPUS is effective coincides with the diseases for which stem cell treatment is used [13]. Therefore, it is generally accepted that LIPUS, in contrast to classical treatment ultrasound, is a non-invasive method of stem cell mechanotherapy in general and regenerative treatment in particular [25, 26, 28].

At the same time, despite the significant potential of LIPUS as a unique treatment factor, and for more than 30 years of research, so far in clinics it is used only to accelerate the healing of bones and soft tissues and accelerate the implantation of dental implants.

The reason for the limited use of LIPUS is the low efficiency of the treatment of diseases. The main physical characteristics of LIPUS signals-vibration frequency, pulse duration, duty cycle and time of their exposure do not differ from classical treatment ultrasound. Only tens and hundreds of times the intensity of treatment signals were reduced, which, along with a large duty cycle, made it possible to avoid their power manifestations-heating, cavitation, acousto-chemical and acoustoelectric effects, which previously masked the manifestations of the effects of low-intensity signals.

There were many attempts, which have been made to improve the effectiveness of treatment with low-intensity ultrasound signals.

In U.S. Pat. No. 5,460,595 it was proposed by Hall et al. the using of several operating frequencies of classical ultrasound to control the depth of exposure and improve treatment results.

Kruglikov I. in DE102011115906A1, 2013, also proposes two or more operating frequencies, which were continuously switched between each other (LDM-Local Dynamic Massage). At the same time, the range of operating frequencies of ultrasound to 16 MHz was expanded.

Vortman, K. in WO 2014135987 A2 proposes to optimize the frequency of ultrasound depending on the type of tissue and the depth of the treatment effect.

Barthe P. et al. in U.S. Pat. No. 8,460,193 B2 describe the system and method of ultra-high-frequency ultrasound treatment, considering it possible to increase the frequency of the treatment signal to 500 MHz to emit it deep into the tissues, the authors proposed a “semi-invasive” introduction using a needle emitter.

To date, preclinical experiments have shown that LIPUS
significantly improves the condition of animals after treatment with chronic myocardial ischemia;
improves the condition after acute heart attacks;
prevents muscle wasting caused by diabetes;
improves cognitive functions of the brain while simulating dementia and Alzheimer's disease;
inhibits the proliferation of breast cancer cells and osteosarcoma;
enhances the growth of osteoblasts and fibroblasts;
strengthens cellular immunity;
accelerates the regeneration of peripheral nerves;
reduces and prevents cerebral ischemia and vascular damage in experimental stroke;
forms new blood vessels and stimulates cellular regeneration in the brain;
improves erectile function;
accelerates osteo-integration of orthopedic implants;
accelerates the healing of bone fractures and wounds.

These diseases are more effectively treated with autologous stem cell injections.

William Tyler radically improved the LIPUS method of treating many diseases, mainly neurological. Starting from the year 2010 and up to the present time [50-77], many modes of operation and parameters of LIPUS ultrasound have been proposed, firstly in the frequency range from 0.02 to 1.0 MHz, and later to 100 MHz, and Ispta intensity from 0.0001 to about 900 mW/cm². Different treatment waveforms are applied, such as harmonic signals, and/or any repetitive impulses or their combinations, such as the stimulus waveforms containing one or many ultrasonic frequencies. Periodically repeating waveforms as a single or plurality pulses are also possible. Each impulse includes from 1 to 50,000 acoustic cycles, repeating with frequencies from 0.001 to 100 kHz, that is, it is emitted in the form of a comb spectrum.

In other words, W. Tyler proposes a low-intensity ultrasound treatment using one modulated harmonic signal or using multiple signals or using multiple comb signals, or combinations thereof. It is obvious that any finite set of harmonic W. Tyler signals is always narrowband, and, therefore, the broadband ratio η<0.2.

W. Tylor uses a patented variety of signaling variants to modulate cellular activity, including nerves and other cells in the human body, namely for changes in ion activity; ion transporter activity;
secreting of signaling molecules;
proliferation of the cells;
differentiating of the cells;
protein transcription of the cells;
protein translation of the cells;
phosphorylation of the cells;
protein structures in the cells or
a combination thereof.

Change in cellular activity leads to a change in the physiological and pathological conditions of organs and tissues and treats the following, but not only: Parkinson's disease, Alzheimer's disease, coma, epilepsy, stroke, depression, schizophrenia, neurogenic pain, cognitive/memory dysfunction, diabetics, traumatic brain injury, spinal/cord injuries, migraine, epilepsy.

The clinical efficiency of W. Tyler signaling treatment is currently being studied. We are not yet aware of commercially available devices based on patents [49-77] or FDA approval for their use. Considering the absence of fundamental differences between Tyler W. signals from the well-studied and approved for clinical use signals from Duarte L., it can be expected that there are ways to further improvement of the efficiency of low intensity ultrasound.

The physical reason for the low clinical efficiency of LIPUS is the discrepancy between the parameters of the ultrasound signal intended to affect cells with those signals that are adequate to the own micromechanical signals of the nuclei of cells, cells and tissues.

It is known that the amount of information that can be transmitted from a source to a receiver is proportional to the frequency bandwidth of the signal and the dynamic range of the signal. When passing from several or from a finite set of narrow-band harmonic signals to a continuous band, that is a very large number of frequencies, it is possible to transmit a much larger amount of information with better noise immunity. For this reason, at the end of the 70s of the last century, ultra-wideband technology for communications and radars appeared which dramatically improved devices the technical characteristics of the devices [78-79].

There have been created radars with the ability to see under ground and through the walls of buildings [11], and noise-immune communication facilities.

The miniature radars began to be used in medicine for remote monitoring of respiration and heartbeat of patients (Mc Ewan T., U.S. Pat. No. 5,573,012 [80]).

Ultra-wideband equipment has appeared for monitoring vital functions of the human body, as well as equipment for cardiological, pneumological and obstetric remote visualization [81].

Mahfouz M. et al. in 2011 described a surgical navigation ultra-wideband system for orthopedics [82].

To date, the development of ultra-wideband radio-frequency systems for imaging and diagnostics of internal organs is being completed [83].

There is known use of ultra-wideband ultrasound in medicine for the purpose of internal organs diagnosing. In ultrasound diagnostics, devices with a frequency band of 3.0-15.0 MHz are already used. Devices with a limiting operating frequency above of 30.0 MHz are under development [84]. In [85] it is shown that very short ultra-wideband ultrasonic diagnostic signals in the form of Gaussian monocycles can propagate in biological tissues with attenuation less than mono frequency, which opens up the prospect of further increasing the upper limit of the operating frequencies of ultrasonic medical equipment.

Ultra-wideband medical devices began to be developed since 1981 by the author of the present invention, A. Marchenko, after the creation by him broadband (later ultra-wideband) ultrasonic multimode transducers with a transducing efficiency comparable to mono frequency ones (Marchenko A. et al, [86-89]).

On their basis, ultra-wideband impact and cosmetology ultrasound devices were created: RU2066215C1, Marchenko A. et al., 1996 and RU2058167C1, Marchenko A. et al., 1996 [91].

In 2014, based on the aforementioned multimode transducers, Tereschenko N. et al. patented (Patent UA 91162) an ultra-wideband ultrasound treatment system known for all of us as ultra-wideband impact devices using a band of ultrasonic frequencies from about 1.0 to about 5.0 MHz in the form of frequency-varying harmonic signals or stochastic continuous signals. The ultrasound intensity from 0.1 to 0.6 W/cm² was chosen.

Limited clinical trials carried out jointly with the author of ultra-wideband technology Marchenko A. at the Kyiv Otorhinolaryngology Institute showed significantly better results in the treatment of chronic tonsillitis compared to classical therapeutic ultrasound [93-94].

Thus, it can be concluded that there are scientific, technical and technological prerequisites for the creation of new ultrasonic/micromechanical signals and devices that provide a stronger and faster course of regenerative processes.

### REFERENCES CITED ABOVE

13. Nassim Rajabzadeh et al. Stem cell-based regenerative medicine. Stem Cell Investigation. 2019; 6.18/http://dx(dot)doi(dot)org/10.2.1037/sci.2019.06.04.
14. Reisman, M.; Adams, K. T. Stem cell therapy: A look at current research, regulations, and remaining hurdles. PTA Peer Rev. J. Formul Manag 2014, 39, 846-857.
15. De Lucas, B.; Perez, L. M.; Galvez, B. G. Importance and regulation of adult stem cell migration. J. Cell. Mol. Med. 2018, 22, 746-754.
16. Fluri D. A. et al. Derivation, expansion and differentiation of induced pluripotent stem cells in continuous suspension cultures. Nat Methods. 2012; 9:509-516.
17. Takahashi K. Yamanaka S. Induction of Pluripotent Stem Cells from Mouse Embrionic and Adult Fibroblast Cultures by Defined Factors/Cell.—2006, V. 126, No 4, pp. 663-676.
18. John B. Gordon, Shinya Yamanaka. The Nobel Prize in Physiology or Medicine 2012.
Caizzo M. et al. Generation of Induced Pluripotent Stem Cells in Defined Three-Dimensional Hydrogels. Method Mol. Biol. 2017; 1612:65-78. Doi 10.1007/978-1-4939-7021-6_5.
20. Shin J. W, Mooney O. J. Improving Stem Cell Therapeutics with Mechanobiology. In Cell Stem Cell, 2016 Jan. 7, 18 (1): 16-19. Doi 10.1016/j stem 2015.12.007.
21. Lim J. M. et al. Somatic cell transformation into stem cell-like cells induced by different microenvironments. Published online, September 2013. https://doi(dot)org/10.4161/org.26202.
22. Haruko Obokata, Charles A. Vacanti et al. Stimulus-Triggered Fate Conversion of Somatic Cells into Pluripotency. 2014. Nature, volume 505, p. 641-647.
23. Sun Y. et al. Mechanics regulates fate decisions of human embryonic stem cells. PLOS one 2012. 7: e37178.
24. Emad Moeendarbary et al. Cell Mechanics: Principles, Practices and Prospects. WIREs Syst Biol Med 2014, 6:371-3888. Doi: 1002/wsbm.1275.
25. Chenyu Huang et al. Mechanotherapy: Revisiting Physical Therapy and Recruiting Mechanobiology for a New Era in Medicine. Trends Mol Med 2013 September; 19 (9), 555-564.
26. G. V. Shivashankar. Mechanics and Genome Regulation. Academ Press, 2019, 200 p.
27. Kirby T. J. and Lammerding. Emerging Views of the Nucleus as a Cellular Mechanosensor. Nat. Cell. Biol. 2018.20:373-381. https://doi(dot)org/10.1038/s-41556-018-0038-y.
28. G. V. Shivashankar. Mechanical regulation of genome architecture and cell-fate decisions. Current Opin. Cell Biol., 56, 115-121 (2019). (Singapore).
29. Beatriz de Lucas et al. Ultrasound Therapy: Experiences and Perspectives for Regenerative Medicine. Genes 2020, 11m 1086; doi: 10.3390/genes 11091086.
30. U.S. Pat. No. 4,530,360. Duarte L. R. Method for healing bone fractures with ultrasound, 1985.
31. U.S. Pat. No. 5,904,659. Duarte L. R. et al. Ultrasonic treatment of wounds. 1999.
32 Xu, P.; Gul-Uludag, H.; Ang, W. T.; Yang, X.; Huang, M.; Marques-Curtis, L.; McGann, L.; Janowska-Wieczorek. Low-Intensity pulsed Ultrasound—medical stimulation of hematopoietic stem/protogenitor cell viability, proliferation and differentiation in vitro. Biotechnol. Lett. 2012, 34, 1965-1973
33. US2006/0241522A1. Chandraratna H. Ultrasound Devices and Methods for Treating Ischemia and Other Cardiovascular Disorders. Oct. 26, 2006.
34. Emanuel Braga Rego et al. Current Status of Low Intensity Pulsed Ultrasound for Dental Purposes. Open Dent J., 2012; 6:220-225.
WO2015/077006. Boer Miriam Sara et al. Skin cancer treatment using low intensity Ultrasound.
US2016/0067526. Feng-Yi Yang. Method for Treating and/or Preventing Neurodegenerative Diseases by using Low Intensity Pulsed Ultrasound (LIPUS).
37. Peng D. Y. et al. Low intensity pulsed ultrasound for regenerating peripheral nerves. Asuan J. Androl. 2020, 22,335-341.
38. US2007/0249938. Shilds. Systems, Devices and Methods Employing Therapeutic

### Ultrasound of Living Tissues.

39. US2010/0204618A1. Min et al. Ultrasound Equipment for Treating of Edema and Use Thereof.
40. U.S. Pat. No. 7,789,841 B2. Huckle J. et al. 2010. Method and Apparatus for Connective Tissue Treatment.
41. Zhou, X. Y.; Zhang, X. X.; Yu, G. Y.; Zhang, Z. C.; Wang, F.; Yang, Y. I.; Li, M.; Wei, X. Z. Effects of Low-Intensity Pulsed Ultrasound on Knee Osteoarthritis: A Meta-Analysis of Randomized Clinical Trials. Biomed Res. Int. 2018.
42. US2010/152626 A1. Schwartz D. Ultrasonic Treatment of Glaucoma.
43. EPGL Medical Invents New Cell Regeneration Technology For Eye Disease. http://www(dot)epglmed(dot)com.
44. Zhou L. X. et al. Low Intensity Pulsed Ultrasound Protects Retinal Ganglion Cell from Optic Nerve Injury Induced via Eyes Associated Protein. Front Cell Neurosci. 2018, 160.
45. U.S. Pat. No. 5,460,595 Hall et al. Multi-Frequency Ultrasound Therapy Systems and Methods. 1995.
46. DE102011115906A1. Kruglikov, Ilja. System for Generating Ultrasonic Waves and Procedures for Configuring an Ultrasonic System. 2013.
47. WO 2014/135987 A2. Vortman, Kobi. Frequency Optimization in Ultrasound Treatment.
48. U.S. Pat. No. 8,460,193 B2. Barthe P. et al. System and Method for Ultra-High Frequency Ultrasound Treatment. 2013.
49. WO 2010/009141A1. Tyler W. Method and Devices for Modulating Cellular Activity Using Ultrasound.
50. US2011/0178441. Tyler W. Methods and Devices for Modulating Cellular Activity Using Ultrasound.
51. US2012/0296241. Tyler W. Ultrasound Neuromodulation for Treatment of Autism Spectrum Disorder and Alzheimer's Disease and Other Dementias.
52. US2012/0289869. Tyler W. Devices and Methods for Modulating Brain Activity.
53. US20120283604. Tyler W. Ultrasound Neuromodulation Treatment of Movement Disorders, Including Motor Tremor, Tourette's Syndrome, and Epilepsy.
54. US2012/0245493. Tyler W. Ultrasound Neuromodulation Treatment of Addiction.
55. US2012/0232433. Tyler W. Ultrasound Neuromodulation Treatment of Tinnitus.
56. US2012/0226091. Tyler W. Ultrasound Neuromodulation Treatment of Pain.
57. US2012/0220812. Tyler W. Ultrasound Neuromodulation for Stroke Mitigation and Rehabilitation.
58. US2012/0083719. Tyler W. Ultrasound-Intersecting Beams for Deep-Brain Neuromodulation.
59. US2013/0281890. Tyler W. Neuromodulation Devices and Methods.
60. US2013/0261506. Tyler W. Ultrasound Neuromodulation Treatment of Post-Traumatic Stress Syndrome.
61. US20130184728. Tyler W. Ultrasound Neuromodulation for Diagnosis and Other-Modality Preplanning.
62. US2013/0144192. Tyler W. Ultrasound Neuromodulation Treatment of Anxiety (Including Panic Attacks) and Obsessive-Compulsive Disorder.
63. US2013/0096469. Tyler W. Ultrasound Neuromodulation Treatment of Obesity and Eating Disorders.
64. US2013/0079682. Tyler W. Ultrasound-Neuromodulation Techniques for Control of Permeability of The Blood-Brain Barrier.
65. US2014/0364774. Tyler W. Ultrasound Neuromodulation for Clinical Effects.
66. US2014/0194726. Tyler W. Ultrasound Neuromodulation for Cognitive Enhancement.
67. US2014/0107533. Tyler W. Ultrasound Neuromodulation Treatment of Attention Deficit Hyperactivity Disorder,
68. US2014/0094720. Tyler W. Methods and Devices for Modulating Cellular Activity Using Ultrasound.
69. US2014/0094719. Tyler W. Ultrasound Neuromodulation Treatment of Schizophrenia.
70. US2014/0088462. Tyler W. Ultrasound Neuromodulation Treatment of Gastrointestinal Motility Disorders.
71. US2015/0025422. Tyler W. Methods and Devices for Modulating Cellular Activity Using Ultrasound
72. US2016/0303402. Tyler W. Methods and Devices for Modulating Cellular Activity Using Ultrasound
73. US2016/0220850. Tyler W. Devices and Methods for Modulating Brain Activity
74. US2016/0001096. Tyler W. Devices and Methods for Optimized Neuromodulation and Their Application
75. US2017/0246481. Tyler W. Devices and Methods for Optimized Neuromodulation and their Application.
76. US2019/0105517A1. Tyler W. Devices and Methods for Modulation Brain Activity.
77. US 2020/0246641A1. Tyler W. Methods and Devices for Modulation Cellular Activity Using Ultrasound.
78. Terence W. Barett. History of Ultra-WideB and (UWB) Radar & Communications. Pioneers and Innovators.—Progress in Electromagnetics. Symposium 20 Jul. 2000.
79. Chernogor I. F. et al. Ultra-wideband signals: theory, simulation and digital processing/In Proceeding. Ultra-wideband and Ultrashort Impulse Signals, Sevastopol, 2006.—pp 32-37.
80. U.S. Pat. No. 5,573,012. Mc Ewan T. E. Body monitoring and imaging apparatus and method, 1996.
81. Staderini E. UWB Radar in Medicine. IEEE Aerospace and Electronic Systems Magazine. Vol. 17, Issue 1, pp 13-18. 2002.
82. Mahfouz M. et al. The Future of Ultra-Wideband Systems in Medicine: Orthopedic Surgical Navigation. In Book: Novel Application of UWB Technologies. 2011. DOI: 10.5772/2050/.
83. Discussion. Ultra-Wideband (UWB). A New Definition of the Future. https://litum.com.2021.
84. Toshiba Medical System Corporation, toshibamedicalsystems.com
85. Baksheeva, I. V. et al. Analysis method of wideband acoustic signals and fields of ultrasonic systems of diagnostics of biological objects. Proc. of XVII Int. Sci., Tech. Conf. on Radiolocation, Navigation, Communication, Voronezh, 2013, v. 3, p. 2428-2437. 84.
86. Copyright certificate No. 1323141 (3267992) dated 21 Apr. 1981. Marchenko A. T., Bakay E. A. Ultrasonic Broadband Transducer.
87. Ostrovsky I., Marchenko A. Variable Thickness Piezoelectric Transducers for Medical Applications./35th Annual Convention of the ALUM, Georgia, USA, 1991, Abstract #4012.
88. Ostrovsky I., Marchenko A. Ultrasonic Instrument for Information Action on Biological Objects and Tissues./J. Ultrasound Med.—1992.—V. 11, #3.—p. S57.
89. Ostrovsky I. V., Zhabitenko M. K., Marchenko O T. Broadband ultrasound emitters. Ukrainian Journal of Medical engineering and Technology 1994, N21, p. 29-35.
90. RU 2066215 C1. Marchenko A. T., Ostrovsky I. V. Ultrasonic Therapeutic Apparatus. Sep. 10, 1996.
91. RU 2058167 C1. Marchenko A. T., Ostrovsky I. V. Device for Ultrasound Therapy and Cosmetology. Apr. 20, 1996.
92. UA 91162. Tereschenko N. et al. Ultrasonic Automated Therapeutic System. 2014.
93. Zabolotny D. I., Karas A. F., Marchenko A. T. and other Clinical and morphofunctional characteristics of the tonsils in patients with chronic tonsillitis after exposure to low temperatures and broadband ultrasound./YII Congress of otorhinolaryngologists of the Ukrainian SSR 4-6 Oct. 1989, Odessa./Kiev, 1989, p. 304-305.
94. Zabolotny D. I, Marchenko A. T. Prospects for the use of broadband ultrasound in otolaryngology./YI Congress of otorhinolaryngologists of the RSFSR. Abstracts. Dokl. 19-21 Sep. 1990, Orenburg, 1990, p. 86-88.

The method according to some embodiments of the present invention comprises applying ultra-wideband micromechanical impact bursts (UMI Bursts) for amplification and acceleration of regenerative processes in a human body to multiple levels of hierarchy of the tissue structures, having dimensions from about 104 mm to about 1.5 mm, for example.

In the method, according to some embodiments of the present invention, micromechanical energy is non-invasively transferred to mechanosensitive elements of pathologically altered tissues and adjacent healthy tissues located next to them, in the form of ultra-wideband micromechanical bursts each of which is formed by at least two signals with different complementary spectra.

The method according to some embodiments of the present invention includes the use of UMI Bursts, the spectra of which contain a very large number of frequencies in at least one ultra-wideband frequency range, and have wideband rate of 0.2 (η≤0.992, which corresponds to the frequency range 1.0-250.0 MHz.

The UMI Bursts may affect many mechanosensitive elements of cellular structures, intercellular and intracellular matrix, mechanosensitive receptors and elements of cell membranes, organelles, nuclei and DNA.

Due to the transfer of additional energy to a multitude of controlling and interacting structures of cells and tissues, the exchange of information within cells, tissues and between them may be improved. There may be an intensification and acceleration of the processes of physiological and reparative regeneration of degraded, diseased old or damaged tissues of human organs.

The method of non-invasive regenerative ultra-wideband burst (Burst Impact) optionally includes one or more of:
generation in one or more ultra-wideband ranges complementary of the main and at least one corrective electrical signal with a broadband rate η≥0.2;
correction of the spectrum of the main signal by summing with the spectrum of the corrective signal;
ultra-wideband transducing of the corrected electrical signal into an impact UMI Burst;
non-invasive input of UMI Bursts into cells and tissues of the pathology area and surrounding tissues;
transferring the energy of UMI Bursts to a plurality of mechanosensitive elements of the cellular hierarchy resonating at a variety of frequencies to improve the exchange of micromechanical signals between cells and within cells;
strengthening, acceleration and synergy of many processes in tissues and cells aimed at their regeneration;
repeated exposure to bursts, for example for 5-40 days, and the accumulation of regeneration factors in pathologically altered, damaged and surrounding tissues to obtain a treatment effect in the regenerative treatment of many pathologies and diseases.
generation in one or more ultra-wideband ranges complementary of the main and at least one corrective electrical signal with a broadband rate η≥0.2; correction of the spectrum of the main signal by summing with the spectrum of the corrective signal; ultra-wideband transducing of the corrected electrical signal into an impact UMI Burst; non-invasive input of UMI Bursts into cells and tissues of the pathology area and surrounding tissues; transferring the energy of UMI Bursts to a plurality of mechanosensitive elements of the cellular hierarchy resonating at a variety of frequencies to improve the exchange of micromechanical signals between cells and within cells; strengthening, acceleration and synergy of many processes in tissues and cells aimed at their regeneration; repeated exposure to bursts, for example for 5-40 days, and the accumulation of regeneration factors in pathologically altered, damaged and surrounding tissues to obtain a treatment effect in the regenerative treatment of many pathologies and diseases.

According to one embodiment of the present method, the main UMI Burst has a first frequency range with the first shape of the frequency spectrum and first intensity, which may mainly exert stress on tissues and intercellular matrix of the treatment area. At the same time at least one second UMI Burst has at least a second shape of the frequency spectrum and second intensity, that when exposed to cells and the intracellular media may mainly stimulate the processes of reprogramming, direct reprogramming, differentiation, proliferation, and cell replacement in the treatment area.

According to one embodiment of the present method, the first UMI Burst is generated at least in the first frequency range, for example, 1.0-10.0 MHz, with a frequency, space and time-averaged intensity in the treatment area of 1.0-300.0 mW/cm².

This first UMI Burst has a repetition rate of 0.05-100.0 KHz.

At least the second UMT Burst is generated in the second frequency range, for example, 10.0-50.0 MHz, with an intensity averaged over frequency, space and time in the treatment area is between 0.0001-300.0 mW/cm², for example. This at least the second impact UMI Burst is repeated at a frequency of 0.05-103 kHz, for example.

At least the third UMI Burst is formed in the third frequency range of 50.0-250.0 MHz, and have a frequency, space and time averaged intensity of burst on the body surface of 0.0001-100.0 MW/cm² and into the treatment area of 0.0001-30.0 mW/cm², for example. This at least the third UMI Burst has a repetition rate of 0.05-103 kHz, for example.

According to another embodiment of the present method, the burst frequency spectra shapes of the main generators are selected from shock signals, stress signals as well as from frequency spectra of rectangular, triangular, trapezoidal signals, or their differentials, and/or Frequency spectra of arbitrary signals or their combinations.

The frequency spectra of the correction signals of the generators are selected from the differentials of the main signals and/or low-cycle sinusoidal signals, damped sinusoidal signals, damped Sinc signals, as well as from Gaussian monocycles and from other known and arbitrary spectra of low-cycle signals or their combinations.

According to still other embodiments of the present method, the UMI Bursts have pulse durations of 5.0-100.0 ns or less.

According to the still other embodiment of the present method, the sequence of the UMI Bursts is incoherent.

According to still other another embodiment of the present method, the sequence of the UMI Bursts is coherent.

According to one embodiment of the present method, the converting of electrical impulses into ultra-wideband micro-mechanical impact bursts (UMI Bursts) are performed by more than one UWB transducer, coupled to the UWB acoustically transparent protector.

According to still another embodiment of the present method, the UMI Burst stimulation of the treatment area and surrounding tissues is performed by a multi-element UWB transducer that generates the micromechanical field, which converging, diverging or dynamically changing in space and/or time.

According to still another embodiment of the present method, the UMI Bursts are applied to the body surface through acoustically transparent and acoustically coupled to each other intermediate medium, such as UWB protector, and UWB contact layer or an extended UWB medium placed between the protector and the body surface.

According to still another embodiment of the proposed method, the forms/parameters of the frequency spectra of UMI Bursts, as well as the treatment procedure, control remotely, including photo and video recording of the procedure, its Internet transmission and documentation.

According to still another embodiment of the present method, the UMI Bursts are introduced into the treatment area through eye conjunctival surface.

According to still another embodiments of the present method, the impact can be interventional and comprise inserting the UMI Bursts into body entity openings, such as nasal cavity, oral cavity, esophagus, rectum, vagina.

According to an embodiment the present method, the UMI Bursts are used in regenerative treatment to treat diseases selected from the group, including at least a stroke, myocardial infarction, ischemia, spinal cord injury, Alzheimer's and Parkinson's diseases, diabetes, some types of cancers, atherosclerosis, varicose, post-traumatic and post-burn scars and wounds.

According to embodiments of the present method, the UMI Bursts are used in regenerative ophthalmology to reduce vision loss, including vision loss due to degradation of the optic nerve or glaucoma.

According to embodiments of the present method, treating UMI Bursts are used in regenerative cosmetology to treat diseases and pathologically altered skin selected from the group, including the signs of skin aging, reduction of scars, acne, wrinkles, post-traumatic and post-surgical seals, melasma, swelling, ptosis.

According to some embodiments of the invention, UMI Burst treatment can be used in combination with ultra-wideband electromagnetic signals, including gigahertz ultra-wideband pulsed radio signals.

The foregoing and other features of the present invention are more fully described below, whereas the following description details only some illustrative embodiments of the invention, but also indicating only some of the different directions in which the principles of the present invention may be employed.

Regenerative restoration of cells and tissues occurs at different levels of cellular organization—molecular, ultrastructural, cellular and tissue [13].

Cellular regeneration is characteristic for the hematopoietic system, skin epithelium, mucous membranes, connective tissue and bones. Such regeneration occurs due to division and subsequent maturation and renewal of cells. There are two phases of cell regeneration-cell proliferation and differentiation.

The combination of cellular and intracellular regeneration may occur during the restoration of the lungs, liver, kidneys, pancreas, endocrine glands, central nervous system.

Intracellular regeneration—renewal and restoration of cells—may prevail in the processes in the myocardium, skeletal muscles and nervous cells.

At the tissue level, in the process of regeneration, the extracellular matrix is renewed, as well as disseminated resident and accumulated pluripotent, and progenitor somatic stem cells are attracted to the treatment area. The stem cells delivered by the blood flow (Homing effect) is involved in the regeneration process.

New cells necessary for regeneration are located in cell niches that are freed from old, diseased and infected cells due to apoptosis—genetically programmed and regulated death by their self-separation into parts that are detected and eliminated by the immune system.

All levels of regeneration are characterized by biochemical regeneration, that is, the renewal of the molecular composition of all organs and tissues of the body.

One basis of regenerative processes is the activity of stem cells of different degrees of differentiation, which in the process of regeneration go through the stages of proliferation, differentiation (maturation), reprogramming (obtaining stem cells from any somatic cells), direct reprogramming (dedifferentiation and transformation into a specialized cell without passing through stage of young stem cells) [95].

At the tissue level, the processes of formation of new functional areas of tissues, vessels and micro vessels are added and formation of tissue innervation systems and numerous receptors. Numerous biochemical regeneration factors are also involved in regenerative processes.

For effective influence on the processes of regeneration, and consequently on a treatment, it may be useful to apply therapeutic factors on many levels of cellular and tissue organization.

Known external and internal factors for increasing the efficiency of regeneration, such as biochemical, physical or the introduction of alien or autologous multiplied stem cells into the treatment area, may only partially solve the problem of accelerating and enhancing the processes, as well as increasing the effectiveness of safe regenerative therapy.

In nowadays, the key role of mechanical/micromechanical signals in controlling the vital processes of intracellular structures, cells and tissues has been established [20-28].

The present invention, in some embodiments, may solve the problem of creating micromechanical impact signals capable of stimulating and enhancing regenerative processes at the tissue, cellular and intracellular levels, and a synergistic increase in the effectiveness of non-invasive regenerative therapy.

The present invention may be implemented into practice with an apparatus having at least one UWB signals generator and at least one UWB signals corrector, as well as one UWB transducer of electrical signals into micromechanical bursts. In addition, the present invention can be implemented by an apparatus having two or more UWB generators, as well as UWB signal correctors and UWB transducers.

Mechanobiology studies show cells and tissues act, sense, decipher and regulate mechanical forces [95]. The effect on tissues and cells of constant or slowly changing forces, as well as mechanical vibrations/ultrasound in detail has been studied [96-101]. The action of impulsive mechanical forces, such as shock or stress waves, as well as a special type of low-intensity perturbations of an elastic medium—micromechanical effects (see Definitions) has been intensively studied.

The main events that occur when mechanical forces interact with tissues and cells are mechanosensitivity, or the ability of cells and tissues to perceive mechanical forces, and mechanotransduction, or the ability of tissues and cells to convert external mechanical signals into biochemical ones in order to stimulate certain tissue and cellular functions that can change their architecture and properties.

The theoretical explanation of mechanobiology is based on the discovery that the extra- and intracellular skeletons of cells act as a dynamic transducer of external forces, concentrating mechanical disturbances and transmitting them to other molecular components outside and inside the cells.

This model of transducing is based on the concept of “tensegrity”—the connection of elements of the extracellular matrix, cytoskeleton and cell structures by tension.

Many oscillatory systems with many natural resonant frequencies that transmit and receive micromechanical disturbances to the structures of tissues and cells are formed. [100,101]

Each resonant system may transmit mechanical signals to higher levels of tissue and cellular organization for their conversion into biochemical ones, to activate certain gene programs and trigger cellular responses.

Mechanical signals propagate through tissue cells through intercellular interactions, which are regulated by special protein complexes, including epithelial cells, endothelial cells, and non-epithelial cells. Their combination coordinates wound healing, tissue remodeling, and morphogenetic development [20-28].

Through the extracellular matrix, mechanical signals control cell adhesion, cell shape and migration, and the activation of downstream signaling pathways to control gene expression, proliferation, and cell fate (species). The matrix consists mainly of collagen, elastin, laminin and fibronectin.

Transmembrane proteins—integrins—transmit mechanical signals into and out of cells and bind the extracellular matrix to the intracellular matrix through their cytoplasmic domains and nanoscale layers, mechanically connecting the domains and the cytoskeleton. The main components of the intracellular matrix are vinculin, paxillin, talin and kinase. The cytoskeleton regulates the propagation of mechanical signals within the cell. It is a dynamic multi resonant structure composed of microfilaments, microtubules and intermediate filaments. Through the cytoskeleton, mechanical signals affect many of the basic and specialized functions of cells.

From the cytoskeletal fibers, mechanical signals collected by integrins are transmitted to the nuclear nucleoskeleton, causing changes in its structure and spatial organization. The structure of the nucleoskeleton is the main regulator of biochemical and physical connections between the nucleus and the cytoskeleton, through which the regulation of gene geometry and gene expression is carried out. Within the nucleus, there are also other multi-resonance structures that connect the nuclear membrane to the cytoskeleton (the so-called LINC complex). Mechanical signals propagating through the LINC complex cause conformal changes in nuclear proteins and directly affect the structure of chromatin and the reprogramming of gene expression.

Thus, biological tissues, cells, extracellular, intracellular, nuclear and intranuclear structures are complex multilevel mechanical pluriresonance systems that conduct mechanical signals inside and outside cells and tissues. It is of fundamental importance that mechanical signals can interact with many different levels of the structural hierarchy of cells, radically influencing different processes of their vital activity, including transformation and expression of genes.

Well known and widely used single-frequency or narrow-band mechanical/ultrasound signals, for example LIPUS, may not be adequate for effective regenerative therapy.

Ultra-wideband low-intensity micromechanical perturbations of the elastic medium (UMI Bursts) may be closer to the multi-resonant plurioscillations of cell and tissue structures and, therefore, are proposed in the present invention for use in regenerative therapy.

Narrowband and ultra-wideband signals differ significantly not only in the frequency band, but also in some other characteristics, for example, information capacity, noise immunity and energy loss (attenuation) during propagation in media.

Information capacity. In narrowband LIPUS technology there is used long pulses with a frequency of 1.5 MHz, with a pulse width of 200 μs, repetitive at a frequency of 1 kHz, with an average spatial and temporal intensity of 1-30 mW/cm². Consequently, LIPUS bandwidth ratio η_{L}≤0.01, LIPUS Band Ratio spectrum width b_{rL}≤0.01, maximum LIPUS spectrum width F_{L}=0.01 MHz, pulse duty cycle T_{L}=0.2, maximum dynamic range of LIPUS signals in the treatment area D_{L}=30.

By UMI Burst technology, ultra-wideband bursts of mechanical energy with duration of 5-100 ns are introduced into the treatment area. Bursts have a continuous spectrum of oscillations in the frequency range FU 1-250 MHz, repeating with a frequency of up to 10³ kHz, with a duty cycle up to TU=1.0 at averaged spatial, temporal and frequency intensities of 0.0001-100 mW/cm², with a dynamic range Du as great as 10⁶.

The amount of information V transmitted from the emitter to the treatment area is [102]: V=F·T·D, and V_{U}/V_{L}=2.5·10⁸ Mbps/0.06 Mbps≈4.1·10⁹ times.

Consequently, amount of information transmitted by new impact UMI Burst signals is several billion times greater than by the known LIPUS signals.

Immunity. Biological tissues are complex structures, within which, when mechanical bursts propagate, there are absorption, scattering on various objects, reflection and re-reflection from internal boundaries and structures, interference of direct and reflected bursts.

Due to the long length of LIPUS harmonic pulses, during their propagation, a lot of reflected signals appear, propagating both along the shortest distance and in many other ways. Multiple dynamic interference of the incoming signals inside the treatment area occurs, which significantly changes the amplitudes, phases and shapes of the LIPUS signals, therefore, influencing the treatment results.

One of the significant potential advantages of UMI Bursts is the absence of interference of directly propagating signals with their reflections from internal tissue structures. A short UMI Burst arrives at the structures of biological tissues in the minimum time, providing the necessary effect, and then leaves the area of influence. Many reflected signals, which are delayed in different ways in time, create some random noise weak signal lagging behind the curative. Thus, the short duration of treating bursts protects them from interference distortions.

Attenuation in an absorbing medium. In it is shown that ultra-wideband ultrasonic low cycle short pulses can propagate in biological tissues with attenuation less than narrowband ones.

This effect is widely used in ultrawideband electromagnetic devices. To date, many ultra-wideband radars have been created, which, due to their ultra-wideband, allow one to “see” objects through walls [103], underground or inside the body [105]. In the past few years, new medical devices for remote diagnostics have been created:
breast tumor detection;
bone cancer detection;
brain hemorrhage detection:
body position and localization:
noncontacting medical imaging;
detection of vascular pressure;
blood glucose concentration level measurement;
octretics imaging radar.

The use of ultra-wideband ultrasonic signals is also known for treatment purposes (inventions of the author of the present invention [90-91]).

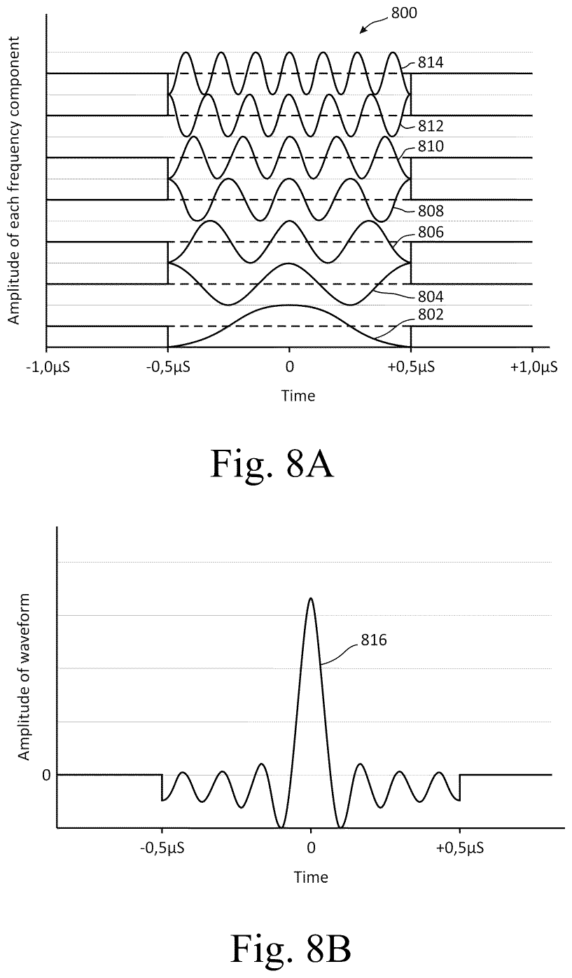

One of the embodiments of the present invention is shown below. The block diagram in FIG. 10 illustrates the method of ultra-wideband micromechanical impact Burst regenerative treatment (UMI Burst treatment), which is implemented using the device 10. The device contains an ultra-wideband main generator 12 and a corrective generator 13, which generate electrical frequency spectrum complementary to spectrum of main generator 12, that together satisfy the value of the (broad-band rate) of 0.2<η<0.992 and the corresponding frequency band 1-250 MHz.

Ultra-wideband signals can have one ultra-wide frequency range formed by generators 12 and 13 or at least two ultra-wide frequency ranges, formed by at least the second main generator 14 and the corrective generator 15. The outputs of generators 12 and 13, as well as generators 14 and 15, are connected to the inputs of ultra-wideband spectrum corrector 16. The coordinated operation of generators 12-15 by controller 18 is controlled, which also performs the functions of a programmer and interface.

The corrector performs band-pass filtering of signals in each frequency range, normalization of their amplitudes and synchronous summation in order to obtain at the output frequency spectra with specified characteristics. The output of the spectrum corrector to the input of ultra-wideband amplifier 20 is connected, and the signals from which are fed to ultra-wideband head 21.

Head 21 contains ultra-wideband transducer 22 of electrical pulses into detached bursts of low-intensity micromechanical energy that is ultra-wideband micro-mechanical impact bursts-UMI Bursts. Transducer 22 is equipped with rear 23 and front 24 electrodes connected to amplifier 20. UMI Bursts through ultra-wideband protector 26 of head 21 and, through the conductive UMI Bursts coupling medium, are delivered to the surface of body 28, and then non-invasively injected into treatment area 30 and adjacent tissues 31.

To deliver a specified amount of energy to the selected treatment area, using controller 18 and functional blocks 12-15, 16, 20, 21-26 of device 10, they select the parameters of UMI Bursts and select the shape of the burst emission field of any shape, suitable to the geometrical form of the treatment area, for example, divergent, collinear, converging or a dynamically changing.

The energy of UMI Bursts is transmitted to a variety of mechano-sensitive resonant tissue structures, including cellular ensembles 31, extracellular matrix 33, somatic cells 34, progenitor cells 36, pluripotent cells 38, and cell nuclei 40.

The UMI Bursts may stimulate and reinforce regenerative processes at various levels of mechano-sensitive tissue structures, including:
apoptosis of deviant cells,
enhancement of many biochemical reactions of tissues and cells that accompany and accelerate regenerative processes,
stress reprogramming of the part of somatic cells into multipotent and progenitor stem cells;
nuclear reprogramming of another part of somatic cells into multipotent and progenitor stem cells;
acceleration and enhancement of natural regeneration.

UMI Bursts promotes accumulation of pluripotent and progenitor stem cells in and around treatment area by multi-day repetitions of UMI Bursts, thereby increasing the effectiveness of treatment.

The electrical ultra-wideband frequency spectra of at least main generators 12 and 14, corresponding to the above electrical signals' shapes of frequency spectra are selected from: shock signals, stress signals, as well as from the frequency spectra of rectangular, triangular, trapezoidal signals or their differentials, and/or from the spectra of arbitrary signals or their combinations.

The electrical ultra-wideband frequency spectra of corrective signals at least of generators 13 and 15 are selected from the differentials of the signals of main generators 12 and 14, as well as from damped sinusoidal signals, damped Sinc signals and from Gaussian monocycles and from all possible spectra of low-cycle signals.

The treating UMI bursts are formed in one ultra-wide frequency range at least from the main and corrective signal, for example, for η>0.2, or are formed from several ultra-wideband frequency ranges, from several complementary signals, for example, for η=0.992.

The intensity Isptaf of UMI bursts is selected from values within the range from 10-5 mW/cm² to 102 mW/cm², at a repetition rate of 0.05-5-102 KHz. The peak value of the burst signal intensity Ipa≤10 W/cm² at a repetition rate of up to 100 Hz.

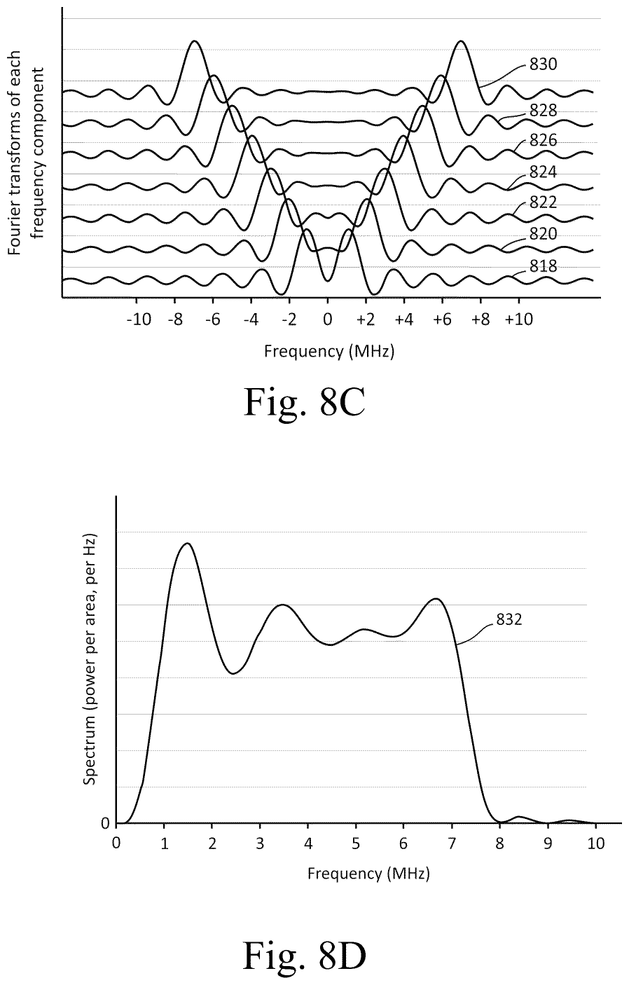

FIG. 11 shows an example of a complementary pair of spectra of treating signal. FIG. 11A is a Gaussian monocycle spectrum 42 for the frequency range 0-50 MHz, FIG. 11B is its corresponding pulse shape 44. In curve 42, after about 20 MHz, the amplitude of the frequency spectrum-decreases significantly with frequency. Corrective spectrum 46 (FIG. 11C) is selected in the form of an exponentially damped sinusoid 48 (FIG. 11D). Its frequency spectra increase with frequency after 20 MHz. After spectrum corrector resulting spectrum 50 is shown in FIG. 11E. It has the necessary for UMI Bursts treatment frequency response. FIG. 11F shows the complex shape of treatment time-domain pulse 52 after spectrum corrector 16.

In accordance with a method of ultra-wideband micromechanical burst treatment, which is implemented in device 10, exposure to ultra-wideband micro-mechanical bursts may initiate and accelerate and intensify many processes that underlie the renewal and regeneration of cells and tissues, including:
reprogramming and transformation of some somatic cells into pluripotent stem and/or progenitor stem cells;
proliferation of a part of somatic cells and acceleration of their division into new mature specialized and stem cells;
change in the state of the extracellular matrix and synergistic acceleration of multilevel reprogramming of somatic cells into stem cells;
accumulation in the treatment area and in the surrounding healthy tissues of scattered “sleeping” and delivered by the bloodstream (homing effect) stem cells;
secretion of biologically active factors and production of proteins that accelerate regeneration processes;
increased apoptosis of pathological cells and acceleration of the release of “niches” for proliferating cells;
proliferation of stem multipotent and progenitor cells, and replacement of damaged and diseased cells with stem cells with their further differentiation into surrounding tissues.

FIG. 12 shows a block diagram of an ultra-wideband experimental facilities based on the certified AIMS III Scanning System (ONDA Corporation, USA).

Absorption of continuous narrow-band ultrasound of different frequencies, broadband stochastic ultrasound, and ultra-wideband micromechanical impact bursts (UMI Bursts) in water were experimentally compared.

The experimental facilities contain ultra-wideband generator 54 connected to ultra-wideband amplifier 56, the signal from which is fed to multimode ultra-wideband transducer 58. Calibrated needle hydrophone 60 and amplifier 62 from Precision Acoustic (UK) in the measurements were used.

Experimental ultra-wideband transducer 58 emitted harmonic ultrasound at frequencies of 1.0, or 3.0 or 5.0 MHz (η<0.01, bᵣ (1.01), or ultra-wideband stochastic signal with a frequency band of 1.0-3.0 MHz (η=1.0, bᵣ=3), or ultra-wideband continuous spectrum UMI Bursts with a frequency band of 1.0-7.0 MHz (η=1.5, bᵣ=7).

Changes in the mentioned signal intensities, depending on the distance of hydrophone 60 from the transducer 58, after preamplifier 62 and analog-to-digital converter 64 on computer 68 was recorded.

FIG. 13 shows the changes in the intensity of ultrasound/micromechanical signals depending on the distance to transducer 58. The figure shows the curves: 70-1.0 MHz, 72-3.0 MHz, 74-5.0 MHz, 76 is a stochastic noise having a band of 1.0-3.0 MHz, 78 is a ultra-wideband UMI Burst with a frequency band of 1.0-7.0 MHz.

As can be seen from FIG. 13, the intensities of a monofrequency harmonic signal with a frequency of 5.0 MHz, and stochastic noise of 1.0-3.0 MHz, decrease with distance with an attenuation coefficient α≈6.25 dB/cm. Signals reach the equipment noise level at a distance of 2.7 cm from the transducer. Monofrequency harmonic signals with frequencies of 1.0 MHz and 3.0 MHz decrease with distance from attenuation coefficient α≈4.5 dB/cm and reach the noise level at a distance of 3.0 cm.

Ultra-wideband UMI Bursts with a frequency range of 1.0-7.0 MHz decrease in amplitude with distance from the emitter much more slowly, on average α≈2.8 dB/cm, and reach the same noise level at a distance of 6, 2 cm from the transducer.

Therefore, ultra-wideband signals can affect biological structures not only on the surface of the body, but also in depth.

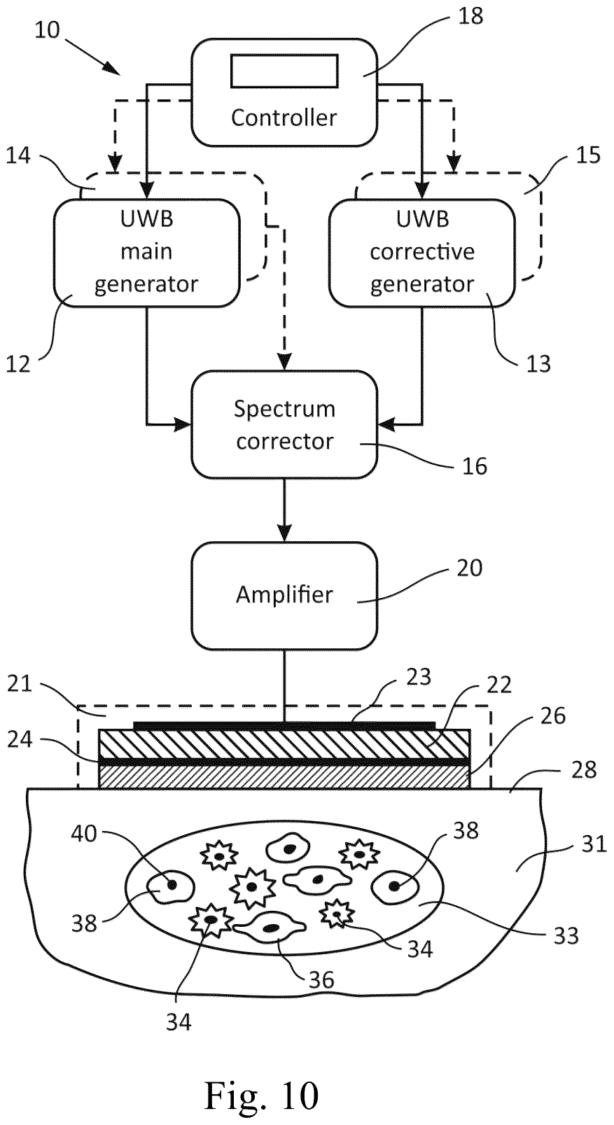

FIG. 14 shows the differences in the impact of ultra-wideband micromechanical bursts (UMI Bursts) with a frequency range of 1.0-7.0 MHz on the proliferation rate of normal mouse fibroblast cells, in comparison with exposure to ultrasound with frequencies of 1.0 MHz, 3.0 MHz and 7.0 MHz.

Studies were performed on normal fibroblasts of BALB/c mice (cell line 3T3-A31).

Ultrasonic harmonic signals with frequencies of 1.0, 3.0 and 7.0 MHz, as well as UMI bursts with an emission spectrum in the 1.0-7.0 MHz band range, were fed to an ultrasound/micromechanical ultra-wideband multimode transducer 22 having the diameter of 20 mm from main 12 and correction 13 outputs of SigLent SDG 6052X generator.

The exposure time was 5 min daily for 5 days and beam-average intensity Isptaf of all signals was set at 30 mW/cm², which was measured according to [106,107].

Trypan blue dye was used (Hy Clone, USA).

An Axiostar Plus binocular microscope (Carl Zeiss, Germany) with a digital camera was used to observe and photo to fix the state of cells. Counting the number of cells in a given volume of the nutrient medium was carried out in the camera Goryaeva (Farmmedteh, Ukraine).

24 hours after the fifth treatment session, the number of live and dead cells and their sums were compared.

As seen from FIG. 14, 1.0 MHz ultrasound suppresses fibroblast proliferation to 62% of control. Cell apoptosis appears to occur, which confirms the findings of the publication [108].

At an ultrasound frequency of 3.0 MHz, the effects of cell proliferation and apoptosis are balanced, and the effect of such ultrasound on proliferation is negligible. At a frequency of ultrasonic irradiation of cells of 7.0 MHz, proliferative processes prevail. On day 6, the number of fibroblasts increased by 20% compared with the control. Fibroblast proliferation is most pronounced when exposed to UMI Bursts with a frequency band of 1.0-7.0 MHz and an intensity of 30 mW/cm². The number of cells after exposure to UMI Bursts increased by almost 40%.

Thus, with an increase in the frequency of ultrasound, the proliferation of fibroblast cells increases, and the greatest increase in proliferative activity with UMI Burst exposure is observed.

The diagram FIG. 15 shows the results of stimulation of cell bioactivity (blast transformation of T-lymphocytes) with LIPUS and UMI Burst exposures.

The reaction of blast transformation of T-lymphocytes is an indicator of the of cellular immunity.

Ultrasonic LIPUS signals with frequencies of 1.5 MHz, as well as UMI Bursts with a frequency spectrum in the 1.0-7.0 MHz band, were fed to ultrasonic/micromechanical ultra-wideband multimode transducer 22 having the diameter of 20 mm from master 12 and correction 13 outputs of SigLent SDG 6052X generator.

The exposure time was 5 min daily for 5 days and beam-average intensity Isptaf of signals was set at 30 mW/cm², which was measured according to [107]. The intensity of UMI Bursts was set at 30 mW/cm² and 1.0 μW/cm².

To set up the reaction under aseptic conditions, 10 ml of blood from a person from a person's vein was taken into a test tube with heparin (25 U/ml). The contents of the tube were mixed carefully and left for 60 minutes in a thermostat at 37° C. to precipitate red blood cells. After incubation in a thermostat, the supernatant plasma layer was enriched by leukocytes and was aspirated into a separate sterile tube and the number of leukocytes in 1 ml was determined. Then the suspension of leukocytes with nutrient medium 199 (containing 200 ml of IU of penicillin and 100 IU of streptomycin in 1 ml) was diluted, so that 1 ml contained 1-2 mil of white blood cells, 20% autologous plasma and 80% of the nutrient medium. The prepared leukocyte suspension was poured into sterile 1 ml vials and there was passed some gas mixture containing 5% carbon dioxide. The vials were placed in a thermostat at 37° C. for 5 days.

In the field of view of the optical microscope, there were calculated the percentage of transformed cells visually different from normal cells.

FIG. 15 shows the results.

It is seen the normal blast transformation of leukocytes BTLNORM=7.4%.

Compared with the control LIPUS has practically no effect on the transformation of lymphocytes, BTLLIPUS=6.9%.

The UMI Bursts with an intensity of 30 mW/cm² increases the BTLumT to 10.6% %, that is, almost one and a half times. The UMI Bursts with a micro intensity of 1.0 μW/cm² increases the BTLMICROUMT=23.5%, that is more than three times.

It can be seen that UMI Bursts are an effective stimulator of the activity of immunocompetent cells.

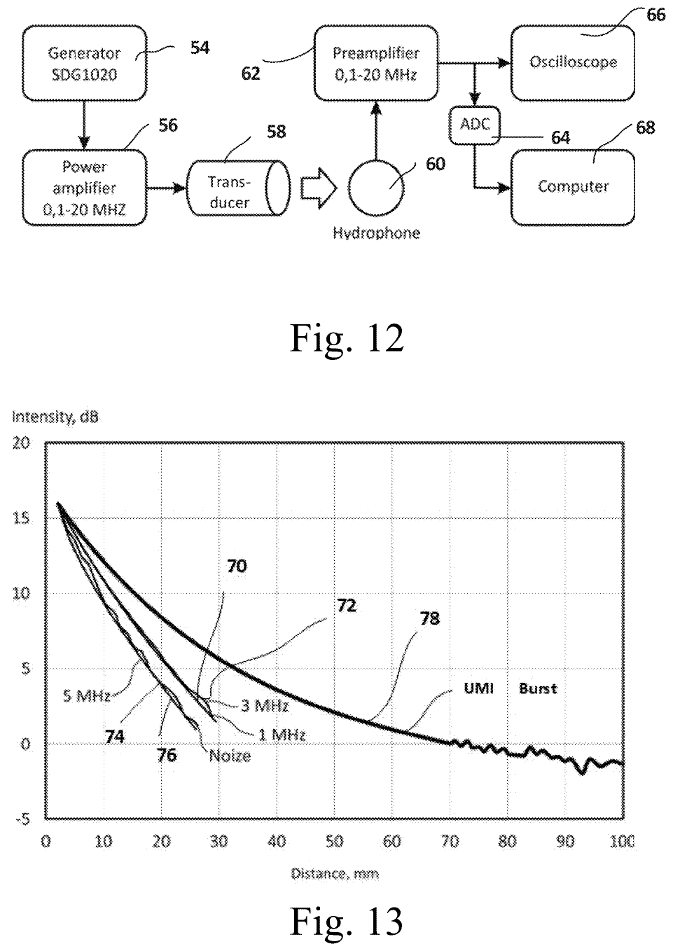

FIG. 16 compares wound healing in the control group of mice, that is, without exposure and, with ultrasonic LIPUS and micromechanical UMI Burst effects.

Studies on 40 mice (female) BALB line were performed. Standardized linear wounds 17 mm long and 1.2 mm deep on their backs were inflicted.

The animals were divided into 3 groups:
a group with classic LIPUS ultrasound impact with an intensity of Ispta=30 mW/cm², a pulse filling frequency of 1.5 MHz, a pulse duration of 200 μs, a repetition rate of 1 kHz.
a group with UMI Bursts with a micro intensity of Isptaf=1 μW/cm², and a radiation spectrum of 1-7 MHz. and a repetition rate of 1 kHz.

Exposure was 5 minutes daily for 9 days until the wounds completely healed in one of the groups.

A beam-average intensity of ultrasonic/micromechanical signals Ispta and Isptaf according to were measured. The intensities of UMI bursts at 30 mW/cm² and 1.0 μW/cm² were set.

We used a SigLent SDG 6052X DDS generator from SigLent Technologies Co. The head for emitting LIPUS ultrasonic and ultra-wideband micromechanical UMI Bursts contained a multimode ultra-wideband transducer with a diameter of 20 mm. Ultrasonic oscillations and micromechanical bursts were delivered to the affected area on the back of the mouse through a layer of ultrasonic gel UBQ 5000 Ultragel.

In the first control group of animals (curve 80), wound healing occurs at the lowest rate, the maximum value of which does not exceed 2.1 mm/day. In the first two days inflammation and edema in this group in the wound area were observed. In the second group of animals after LIPUS exposure (curve 82), inflammation and edema were absent. The healing rate was approximately equal to the healing rate in the control group also is 2.1 mm/day. In the third group of animals, the wounds of which were exposed to UMI bursts (curve 84), the healing rate was significantly higher and reached a value of 3.8 mm/day. Complete healing of wounds in UMI Bursts group was achieved, on day 9 from the start of exposure, when LIPUS treatment was applied, the wound size still was 3 mm on day 9, and in the control group wound size was still 4 mm.

Therefore, UMI Bursts may be more effective in promoting wound healing in compare with LIPUS.

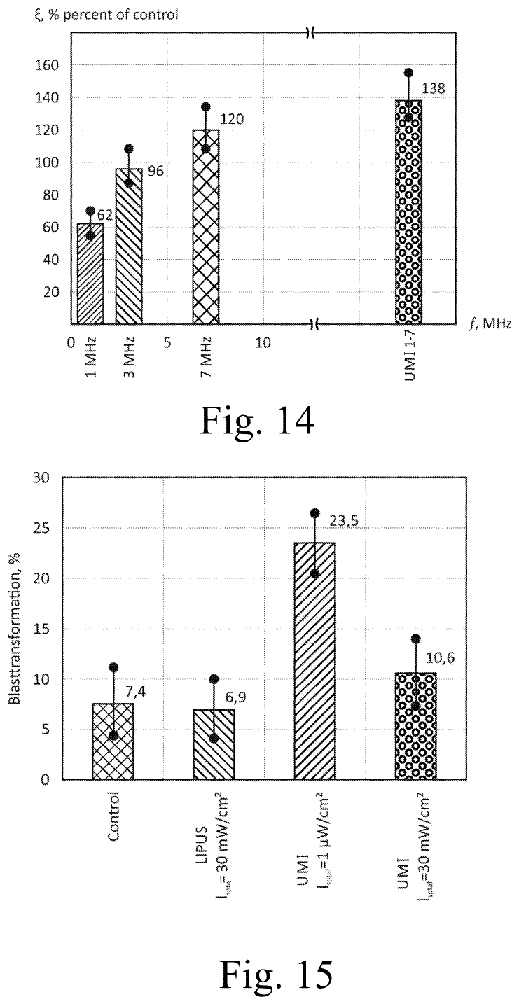

FIGS. 17A to 17C show a variant of the implementation of the UMI Burst method for reducing an old, rough, extensive scar on the face.

The scar of patient Ch., 42 years old man (Kyiv, Ukraine). When he was 3 years old, being hit by the butt of a car door, he was injured. At the age of 42, the length of the scar on the face was 4.1 cm before testing (FIG. 8A).

The SigLent SDG 6052X DDS generator from SigLent Technologies Co was used. The head for emitting UMI Bursts contained a multimode ultra-wideband transducer with a diameter of 10 mm. The UMI Bursts to the scar and surrounding skin were delivered through a layer of ultrasonic gel UBQ 5000 Ultragel.

The scar treating area was irradiated with UMI Bursts with a frequency band of 1.0-7.0 MHz, intensity Isptaf=30 mW/cm² and a repetition rate of 1 kHz.

The exposure was carried out daily for 9 days. The duration of each session was 10 min.

FIG. 17A is a photo a pronounced scar with traces of surgical intervention before UMI Burst procedures.

FIG. B is a photo of the scar after 5 days of daily treatments. The traces of sutured wound are almost invisible. The depth of the scar has decreased significantly.

FIG. 17C is a photo of the scar after 9 days of applying UMI Bursts The scar has drastically decreased and become less noticeable. Its length by about 1 cm was also reduced.

There were obtained the regenerative restoration of the skin, similar to the restoration by the stem cell transplantation.

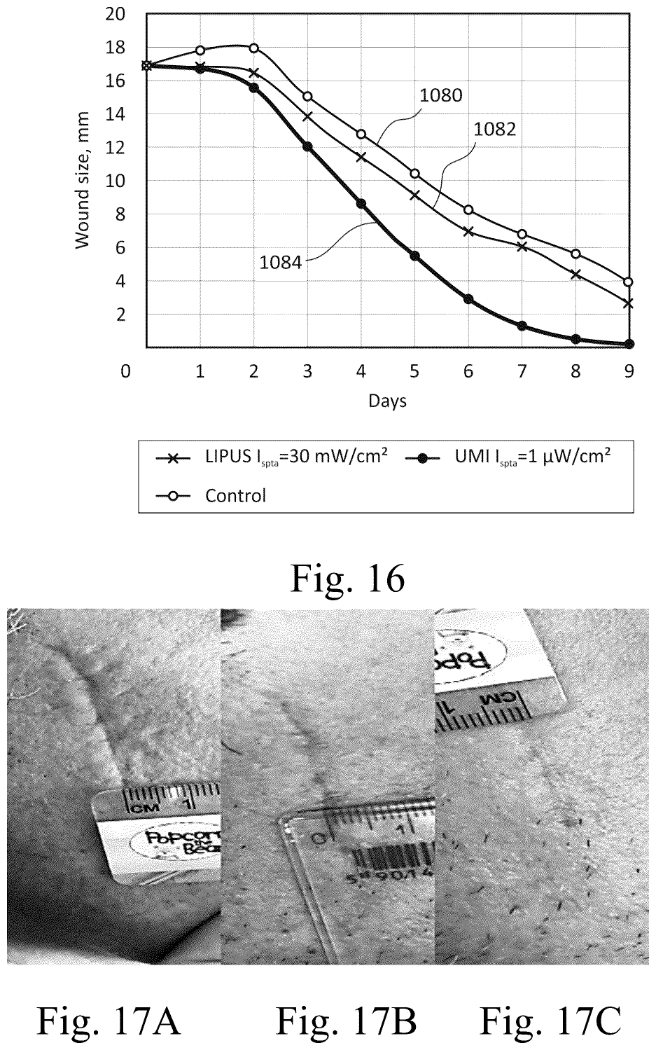

FIGS. 18A and 18B show implementation of the UMI Burst method for drastic reduction of long-term dermal hyperpigmentation (dermal melasma) and reduction of deep forehead wrinkles on the face, respectively.

The UMI Burst signals to the forehead of patient S., 46 years old woman, were applied. To exclude primary biliary cholangitis, hemochromatosis and Addison's disease, before using the UMI Bursts, patient S. underwent the necessary examination in the clinic. According to the conclusion of dermatologists, patient S. had a mixed type of melasma, i.e. both epidermal and dermal. Therefore, the previous long-term treatment was ineffective.

A selected area of the skin with age-related changes with the UMI Bursts with a bandwidth of 1.0-7.0 MHz, with an intensity of Isptaf=30 mW/cm² and a repetition rate of 1 kHz were irradiated. The exposure was carried out daily for 40 days. The duration of each session was 10 minutes. The micromechanical transducer was moved slowly on the affected area.

We used the SigLent SDG 6052X DDS generator from SigLent Technologies Co. The head for emitting the UMI Bursts contained a multimode ultra-wideband transducer with a diameter of 10 mm. The UMI Bursts to the affected area through a layer of ultrasonic gel UBQ 5000 Ultragel were delivered.

From the comparison of FIG. 18A and FIG. 18B, it can be seen that as a result of treatment, the intensity of hyperpigmentation has significantly decreased. The spots are almost imperceptible (visible in the photo only after increasing the contrast of the image FIG. 18B up to 400%.

Intractable deep wrinkles on the forehead also decreased significantly, the skin began to look younger.

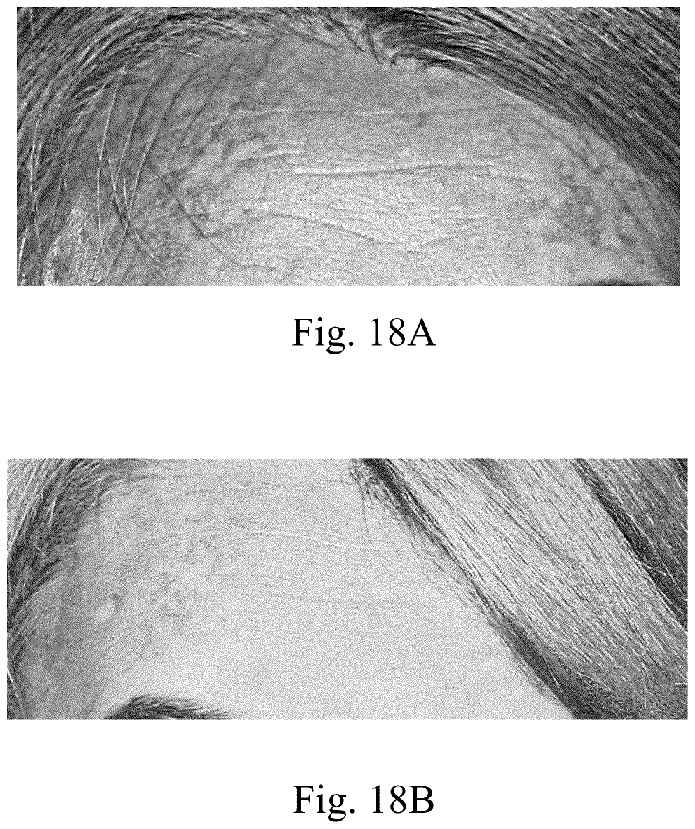

On FIG. 19, the variant of the implementation of the UMI Burst method in cosmetology for correction of nasolabial folds is shown. Quantitative control of the size of creases by using Altera 3D apparatus was made. Testing subject R is a 49 years old woman.

The UMI Burst exposure was used with a frequency band of 1.0-7.0 MHz, with an Isptaf intensity of 30 mW/cm2 and a repetition rate of 1 kHz. The exposure daily for 14 days was performed. The duration of each session was 10 minutes. The micromechanical head was slowly moved on the affected area.

We used the SigLent SDG 6052X DDS generator from SigLent Technologies Co. The head for emitting the UMI Bursts contained a multimode ultra-wideband transducer having diameter of 10 mm. The UMI Bursts were delivered to the treatment area through a layer of ultrasonic gel UBQ 5000 Ultragel.

FIG. 19 shows an example of the correction of the nasolabial folds of the woman R, age 59 years.

Prior to the UMI Burst exposure, the crease volume measured by the Altera apparatus was 10.29 mm³.

After exposure, the volume of the crease decreased to 3.1 mm³, i.e., by 70%. No filling substances under the room were introduced.

The patient was observed by us throughout one year. The effect of improving the appearance was persistent.

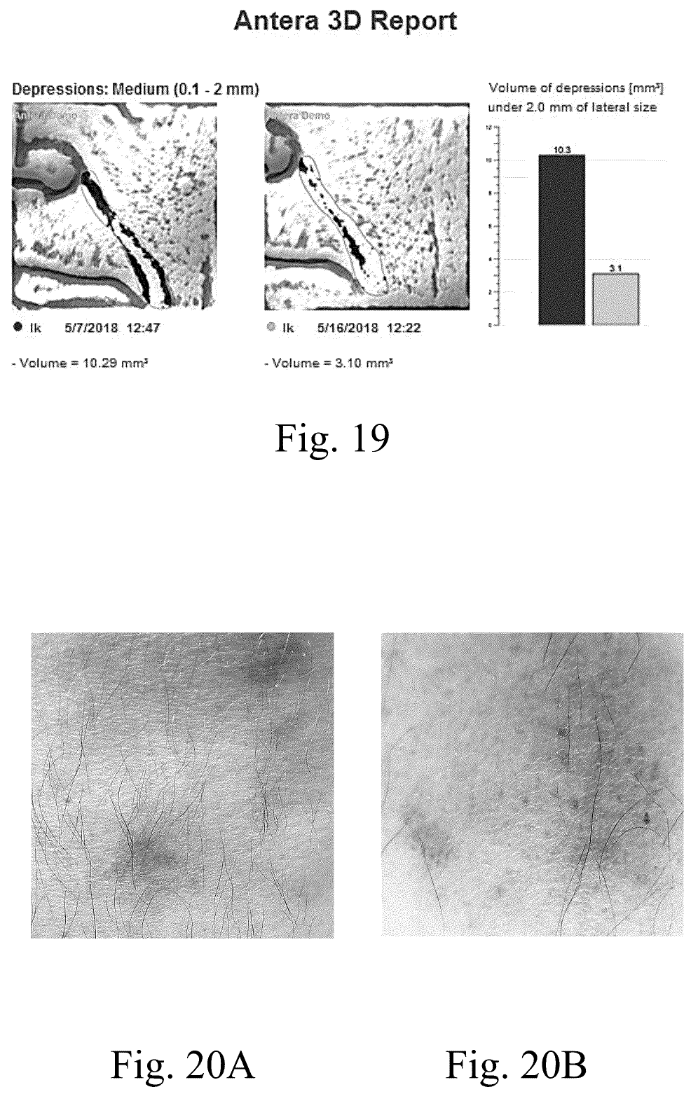

FIGS. 20A and 20B illustrate the implementation of the proposed method for restoring blood microcirculation in the area of the lower limb with varicose veins.

The tests were carried out on the volunteer M, 72 years old man.

The UMI Burst effect was applied with a frequency band of 1.0-7.0 MHz, with an intensity of Isptaf=30 mW/cm² and a repetition rate of 1 kHz. Exposure daily was carried out for 5 days. The duration of each session was for 10 minutes. The micromechanical transducer was moved slowly on the affected area.

We used the SigLent SDG 6052X DDS generator from SigLent Technologies Co. The head for emitting of the UMI bursts contained a multimode ultra-wideband transducer with a diameter of 10 mm. The micromechanical bursts to the treatment area were delivered through a layer of ultrasonic gel UBQ 5000 Ultragel.

The appearance of the shin area with hematomas on the photographs of FIG. 20A and FIG. 20B before and after the UMI Burst treatment is shown. Note that by the time of treatment, hematomas were constantly present in the part of the limb shown in the photographs for more than 4 months, and were accompanied by microcirculatory disorders by periodic severe pain and night cramps of the calf muscles.

After the UMI Burst, exposure already on the fourth day the hematoma almost disappeared. There was a slightly noticeable pigmentation of the skin. Periodic pains and night cramps in the limb also ceased. Observation over 1.5 years showed that the treatment effect is persistent, hyperpigmentation in the observed area no longer occurred.

Thus, in accordance with the proposed method of treatment, with some of the above-described embodiments and applications of the ultra-wideband, micromechanical UMI burst regenerative treatment; it is possible to treat skin diseases, injuries, degenerative and traumatic diseases of the musculoskeletal system and blood vessels circulation disorders. It is also possible to treat eye diseases, lesions of the heart to correct the state of the immune system, stimulate the defenses and general endurance, and on this basis, prevent premature aging. The invention can also be applied in field military and sports medicine, aesthetic medicine and cosmetology.

It is obvious that the present invention is not limited to the above-mentioned embodiments, and variations and modifications may be made without departing from the scope of the present invention. It will be appreciated by persons skilled in the art that the present invention is not limited by the drawings and description hereinabove presented. Rather, the invention is defined solely by the claims that follow.

### REFERENCES CITED IN THE DESCRIPTION ABOVE

95. Jacques E. et al. The Progression if Regenerative Medicine and its Impact on Therapy Translation. Clin Trans Sciences 2020. Clinical and Translational Science 13(3). DOI: 10.1111/cts.12736.
96. Chiara Argentati et al. Insight into Mechanobiology: How Stem Cells Feel Mechanical Forces and Orchestrate Biological Functions. Int. J. Mol. Sci. 2019, 20, 5337; doi; 10.3390/ijms.2021.5337
97. Verbruggen S. Mechanobiology in Health and Disease; Elsevier: Amsterdam, The Netherlands, 2018: ISBN 9780128129524
98. Sun Y. et al. Mechanics regulation fate decisions of human embryonic stem cells. PLOS One 2012, 7: e 37178.
99. Huang Chenyu et al. Mechanotherapy: Reviewing Physical Therapy and Recruiting Mechanobiology for a New Era in Medicine. Trends in Molecular Medicine. Volume 19, Issue 9. p 555-564, 2013.
100. Emad Moeendarbary et al. Cell mechanics: principles, practices and prospects. WIREs Syst Biol Med 2014, 6:371-388.doi: 10.1002/wsbm 1275.
101. Ingber D. E. Tensegrity-based mechano-sensing from macro to micro. Prog. Biophys. Mol. Biol. 2008, 97,163-179
102. Khokhlov G. I. Fundamentals of information theory. Academy Publishing House, 2017.
103. Li, J. et al. Through-wall detection of human being's movement by UWB radar. IEEE Geosci. Remote Sens. Lett. 2012, 9.1079-1083.
104. Oyan M. J. et al. Ultrasound Gates Step Frequency Ground-Penetrating Radar. Geoscience and Remote Sensing. IEEE Trasactions, vol. 50, No. 1 p, 212-222. January 2012.
105. Abdulhameed Habeeb Alghanimi. Medical Application of Ultra-wideband Technology. Published: Jul. 28, 2021. DOI: 10.5772/intechopen. 93577
106. Johns, L. D. et al. Analysis of effective radiating area, power intensity and field characteristics of ultrasound transducers./Arch Phis Med Rehabil 88 (1), 2007, p. 124-129.
107. American National Standard E-1065-99 Ultrasonics. Phisiotherapy systems.
108. Mingfang Shi et al. Low intensity-pulsed ultrasound induced apoptosis of human hepatocellular carcinoma cells in vitro. Ultrasonics, volume 64, January 2016, pages 43-53, https://doi(dot)org/10.1016/jultras.2015.07.011).
109. http://miravex(dot)com/antera-3d

### EXAMPLES

Following is a description of some examples:

1. A method of ultra-wideband micromechanical modulating of cellular activity comprising:
the generation of at least one pair of complementary electrical UWB signals, consisting of a main and correction signals with different distribution of frequency spectra, intended for at least one UWB frequency range of treating signals;
the correction of UWB frequency spectra of the main signals in at least one frequency range by using bandpass filters as well as summation with the spectra of correcting signals, to obtain ultra-wideband electrical signals with constant, rising or arbitrary shape of the frequency spectrum;
converting corrected UWB electrical signals by electromechanical transducer into Ultra-wideband Micromechanical Impact Bursts (UMI Bursts);
delivery of impact UMI Bursts through an acoustically transparent UWB protector and a UWB coupling medium, which conduct UMI Bursts to the surface of object;
non-invasive injection and delivery of impact UMI Bursts to the treatment area and adjacent to the treatment area of the biological tissues;
UMI Burst accumulation of pluripotent and progenitor stem cells in and around treatment area by multi-day repetitions of treating by the UMI Bursts;
transfer of energy of impacted the UMI Bursts to variety of mechanosensitive structures of the biological tissues in the treatment area and adjacent tissues, including cell assemblies, intracellular and extracellular matrices, cells, organelles and cells' nuclei;
stress impact on cells and extracellular matrix to apoptosis stimulation of deviant cells;
stimulation and enhancement of many biochemical reactions of tissues and cells that accompany and accelerate regenerative processes;
stress reprogramming of the part of somatic cells into pluripotent and progenitor stem cells;
nuclear reprogramming of another part of somatic cells into pluripotent and progenitor stem cells,
stimulation, acceleration and enhancement of natural regeneration.
h) ultra-wideband micromechanical impact burst (UMI Burst) stimulation of regenerative processes at various levels of mechanosensitive tissues and cell structures, including:
stress impact on cells and extracellular matrix to apoptosis stimulation of deviant cells;
stimulation and enhancement of many biochemical reactions of tissues and cells that accompany and accelerate regenerative processes;
stress reprogramming of the part of somatic cells into pluripotent and progenitor stem cells;
nuclear reprogramming of another part of somatic cells into pluripotent and progenitor stem cells,
stimulation, acceleration and enhancement of natural regeneration.

2. The method according to example 1, in which the first UMI Burst has a first frequency range with a first shape of frequency spectrum, which exerts stress on tissues and intercellular matrix of the treatment area, and wherein at least the second UMI Burst has at least a second frequency range with a second shape of frequency spectrum, that stimulates the processes of reprogramming, direct reprogramming, differentiation, proliferation and cell replacement in the treatment area of diseased or damaged cells with healthy ones.

3. The method according to example 2, wherein the first of said UMI Bursts is generated at least inside the first frequency range 1.0-10.0 MHz, with a frequency, space and time-averaged intensity in the treatment area of 1.0-300.0 mW/cm².

4. The method according to example 3, wherein the first of said UMI Bursts has a repetition rate of 0.05-100.0 kHz.

5. The method according to example 3, wherein at least the second of said UMI Bursts is generated inside the second frequency range of 10.0-50.0 MHz, with an intensity averaged over frequency, space and time in the treatment area of 0.0001-30.0 mW/cm².

6. The method according to example 5, in which at least the second of said UMI Bursts is repeated at a frequency of 0.05-103 kHz.

7. The method according to example 2, wherein, at least the third of several UMI Burst is formed inside the third frequency range of 50-250 MHz, and have a frequency, space and time averaged intensity of burst on the body surface of 0.0001-100 MW/cm² and into the treatment area of 0.0001-30 mW/cm².

8. The method according to example 7, wherein said at least the third UMI Burst has a repetition rate of 0.05-103 kHz.

9. The method according to example 1, in which the frequency spectra of the main signals of the generators are selected from shock signals, stress signals, as well as from the frequency spectra of rectangular, triangular, trapezoidal signals or their differentials, and/or from the frequency spectra of arbitrary signals, or their combinations.

10. The method according to example 1, in which the frequency spectra of the correction signals of the generators are selected from the differentials of the main signals, and/or from low-cycle sinusoidal signals, damped sinusoidal signals, damped Sinc signals, as well as from Gaussian monocycles, and from other known and arbitrary spectra of low-cycle signals or their combinations.

11. The method according to example 1, in which UMI Burst has a pulses duration of 5.0-100.0 ns.

12. The method according to example 1, wherein the sequence of UMI Bursts is incoherent.

13. The method according to example 1, wherein the sequence of UMI Bursts is coherent.

14. The method according to example 1, in which the transducing of electrical impulses into UMI Bursts perform by more than one UWB transducer, acoustically coupled to the UWB acoustically transparent protector.

15. The method according to example 14, wherein the UMI Burst stimulation of the treatment area and surrounding tissues is performed by a multi-element UWB transducer that generates the UMI Burst field, converging, diverging or dynamically changing in space and time.

16. The method according to example 1, wherein said UMI Bursts are applied to the body surface through acoustically transparent and acoustically coupled to each other intermediate medium, such as UWB protector, and UWB contact layer or an extended UWB medium placed between the protector and object's surface.

17. The method according of examples 1-16, wherein parameters of the spectra of impact UMI Bursts, as well as the treatment procedure, control remotely, including photo and video recording of the procedure, its Internet transmission and documentation.

18. The method according to examples 1-16, wherein said UMI Bursts are introduced into the treatment area through eye conjunctival surface.

19. The method according to examples 1-16, wherein the treatment can be interventional and comprise inserting the impact UMI Bursts into body entity openings, such as nasal cavity, oral cavity, esophagus, rectum, vagina.

20. The method according to examples 1-16, wherein said UMI Bursts are used in regenerative treatment to treat disease is selected from a group, including at least a stroke, myocardial infarction, ischemia, spinal cord injury, Alzheimer's and Parkinson's diseases, diabetes, some types of cancers, atherosclerosis, varicose, burns, post-traumatic and post-burn scars and wounds.

21. The method according to examples 1-16, in which the UMI Bursts are used in regenerative ophthalmology to reduce vision loss, including, at least vision loss due to degradation of the optic nerve including glaucoma.

22. The method according to examples 1-16, wherein said UMI Bursts are used in regenerative cosmetology to treat diseases and pathologically altered skin selected from the group, including—the signs of skin aging, reduction of scars, acne, wrinkles, post-traumatic and post-surgical seals, melasma, swelling, ptosis.

23. The method according to example 1, additionally including the use of the UMI Bursts in combination with UWB radio frequency pulse signals of the gigahertz frequency range.

### Section 2

The present invention, in some embodiments, relates to medicine, particularly to non-invasive regenerative therapy, and more specifically, to devices and methods of their operation for the restoration of diseased or damaged tissues of the human body by ultra-wideband micromechanical frequency burst action on biological tissues and cells in order to enhance and accelerate the processes of their regeneration and repair.

The action is carried out on various levels of organization of tissue and cellular activity, including mechanosensitive structures of tissues and cells, including cellular ensembles, extracellular, intracellular, intranuclear matrices, blood vessels, blood cells, cells of nervous system, cells of the immune system, specialized somatic cells, stem cells, biochemical factors accompanying cellular activity, cell nuclei and DNA.

During exposure, natural reparative processes are induced and accelerated, including programming, reprogramming and transformation of somatic cells into progenitor, multipotent and pluripotent stem cells, accumulation of these cells and accelerated safe replacement of old cells with new ones.

### Regenerative Therapy

Regenerative therapy considers means and methods of treatment, including cellular technologies, that promote regeneration or the formation of new tissues in damaged or pathologically altered tissues and organs. Regenerative restoration of tissue structure occurs at different levels of their organization-molecular, intracellular, cellular, tissue, organ. Recovery is carried out using cellular or intracellular hyperplastic processes.

All biological structures undergo physiological regeneration. Where the cellular form of regeneration dominates, cell renewal takes place. In tissues and organs where the cellular form of regeneration has been lost, intracellular structures are renewed. Along with the renewal of cells and subcellular structures, biochemical regeneration is constantly taking place, i.e., the renewal of the molecular composition of all cells, tissues and organs without exception.

Structural and functional specialization of organs and tissues determines for some the predominantly cellular form, for others—predominantly or exclusively intracellular, for the third—equally both forms of regeneration. Organs and tissues in which the cellular form of regeneration predominates include bones, skin epithelium, mucous membranes, hematopoietic and loose connective tissue, etc. Cellular and intracellular forms of regeneration are observed in glandular organs (liver, pancreas, endocrine system), lungs, smooth muscles, autonomic nervous system. The organs and tissues where the intracellular form of regeneration predominates include the myocardium. In the central nervous system, this may be the only form of regeneration.

Physiological regeneration is the restoration of cell and tissue elements after their natural death. The mechanisms of physiological regeneration in different tissues of the body are somewhat different, therefore, several groups are distinguished on this basis:
group I skin epidermis, intestinal epithelium, blood cells, loose connective tissue. They regularly alternate between death and replacement of dead cells by new dividing cells. These tissues are characterized by the presence of stem cells and their high mitotic activity.
II group of tissues, which combines cellular and intracellular regeneration—this group includes the epithelium of the liver, kidneys, lungs and endocrine glands, smooth muscle tissue. These tissues also contain stem cells, but they normally do not divide as often as in group I.
III group of tissues is characterized only by an intracellular form of physiological regeneration: striated tissue of the cardiac type, nervous tissue, epithelium of the pancreas and salivary glands. In the tissues of group III, stem cells are practically absent; therefore, physiological regeneration proceeds by constant renewal of the organelles of mature cells. The fabrics of this group are sometimes called “eternal fabrics”. All 4 types of biological tissues of the body can regenerate—connective, epithelial, muscle and nervous.

Reparative regeneration consists of two phases—proliferation and differentiation. The first phase is the reproduction of young undifferentiated stem cells. Each tissue is characterized by its own stem cells, which differ in the degree of proliferative activity and specialization. The second phase is differentiation: young stem cells mature, their structural and functional specialization takes place, and they compensate for the loss of highly differentiated cells. The same change of hyperplasia of ultrastructures by their differentiation underlies the mechanism of intracellular regeneration. The source of regeneration can be highly differentiated cells of the organ, which, under the conditions of a pathological process or external influence, can transform into stem cells, acquiring the ability for mitotic division with subsequent differentiation.

Immediately after damage, reactive changes develop in the tissues, proliferation, differentiation and integration of cells are disturbed. If damaged cells do not adapt to new conditions, their decay, death and elimination occur. Forms of manifestation of regenerative histogenesis (for example, cell reproduction or hyperplasia of intracellular structures) are specific for each tissue. In renewing tissues, for which normal histogenesis is characterized by cell proliferation by mitosis, the main role in regeneration processes belongs to the mitotic division, primarily of stem cells, including stem cells attracted to the pathology zone (Home effect). Regeneration histogenesis of growing tissues occurs both in the processes of cell proliferation and intracellular increase in structural components (organelles).

In a damaged organ, the regeneration process includes a complex of interstitial and intercellular interactions of structures. Intercellular and intracellular matrices are formed. There are processes of interaction between the epithelium, connective and nervous tissues. To a large extent, the outcome of the recovery process is determined by inflammatory growths of the connective tissue. All regenerative processes take place in the interaction of the nervous, endocrine, vascular, immune systems with new tissues being formed. A system of blood supply and innervation of tissues is being created.

### Definitions

For the purpose of this invention, the following terms employed herein refer to the following concepts:
“Regenerative therapy”—restoration of diseased or damaged tissues and human organs—using pluripotent, multipotent and progenitor stem cells, activated, transplanted or transformed [1];
“Regenerative cosmetology”—a technology for rejuvenating (revitalizing) aging skin by activation or transplantation of stem cells and induced transformation of somatic cells into stem cells [2];
“Micro-energy regenerative therapy”—the impact in the treatment area of signals with a maximum peak energy less than 0.1 mJ/mm², and a spatial-peak temporal average intensity of less than hundreds of microwatts per square millimeter [3].
“Induced stem cells”—pluripotent multipotent and progenitor stem cells obtained from somatic cells by epigenetic reprogramming;
“Ultra—wideband (UWB) signals”—the fractional bandwidth n and the frequency band ratio bᵣ of signals which correspond to the following values:

$$η = 2 \frac{{f}_{h} - {f}_{l}}{{f}_{h} + {f}_{l}} + 2 \frac{{b}_{r} - 1}{{b}_{r} + 1} \geq 0 , 2 ,$$

where fₕ and fₗ are the upper and lower frequencies of the spectrum of signal at the level −3 dB, bᵣ is the ratio:

$${b}_{r} = \frac{{f}_{h}}{{f}_{l}} = \frac{2 + η}{2 - η} \geq 1 , 22.$$

In accordance with the Standards [4-10] and scientific research [11-12] the fractional bandwidth η and the frequency band ratio bᵣ:

| | |
|---|---|
| Fractional Bandwidth η | Band Ratio bᵣ |

Narrowband

| | |
|---|---|
| 0.00 < η ≤ 0.01; | 1.00 < bᵣ ≤ 1.01, |

Wideband

| | |
|---|---|
| 0.01 < η < 0.2 | 1.01 < bᵣ ≤ 1.22 |

Ultra-wideband

| | |
|---|---|
| 0.2 < η < 2.00 | 1.22 < bᵣ < ∞ |

“Ultra-wideband Micro-mechanical Frequency Burst (UMB)”—moving in medium detached ultra-wideband micro-mechanical frequency disturbance of the medium, which differs in frequency and spatial characteristics from narrowband and wideband ultrasound;
Oscillatory modes of an electromechanical transducer”—a set of natural or forced vibrations of a transducer which different by physical characteristics, for example: resonance modes—radial R, edge E, angular A, volume V, as well as planar ultra-wideband P—mode vibration of the surface layer of the transducer;
“Ispta—narrowband signal intensity”—spatial-peak temporal-average intensity of narrowband signal or the sum of narrowband signals, averaged over the cross section of the ultrasonic beam, impulse rate and over time.

“Isptaf intensity of wideband and ultra-wideband signals, in particular, ultra-wideband micro-mechanical frequency bursts (UMB)”—the spatial-peak temporal-average intensity averaged over cross-section of the burst radiating beam, as well as over time, impulse rate and over burst spectrum.

### REFERENCES CITED IN THE DEFINITIONS ABOVE

Atala (editor) et al. Principles of Regenerative Medicine, Academic Press, 2019, 1416 pages.
Sucharita Boddu et al. Regenerative Medicine in Cosmetic Dermatology. Review. Cutis. 2018 January; 101 (1): 33-36.
13. Yegang Chen et al. Role and Mechanism of Micro-energy Treatment in Regenerative Medicine. Translational Andrology and Urology. Feb. 8, 2020 doi org/10.21037/tau.2020.02.25
14. OSD/DARPA, Ultra-Wideband Radar Review Panel,” Arlington, VA, Defense Advanced Research Project Agency (DARPA), 1990.
15. US Federal Communication Commission (FCC), Part 15, October 2003, http://www(dot)fcc(dot)gov/oct/info/rules.
16. EC 2009 Commission of the European communities Decision 2007/131/EC April 2009.
17. International Electrotechnical Commission (IEC), Basic EMC Publication 6100-2-13: “Environment-High-power Electromagnetic (HPEM) Environment-Radiated Conducted”. American National Standards Institute (ANSI) ANSIC63.14-1998, American National Standards
18. Institute of Electrical and Electronics Engineers (IEEE), IEEE Std 6861997, IEEE Standard Radar Definitions, 16 Sep. 1997.
19. Oyan, M. J.; et al. “Ultrasound Gates Step Frequency Ground-Penetrating Radar”. Geoscience and Remote Sensing, IEEE Transactions, vol. 50, No. 1, pp. 212-22, January 2012
20. F. Sabath et al. Definition and Classification of Ultra-Wideband Signals and Devices. Radio Science Bulletin (2005), No 313 pp. 12-20.

In 1909 year, the term “stem cells” was introduced [11]. With a glance of function within any live organism contemporary biologists discriminate:
Totipotent (omnipotent) stem cells, which can originate a full-value viable organisms by differentiation up to formation of all embryonic and adnexa tissues in the form of three-dimensional mutually-connected structures (such stem cells include a zygote and several its descendants obtained during some first cycles of cell-division);
Pluripotent stem cells, which are direct or remote descendants of the totipotent cells, can originate almost all tissues and organs (with the exception of extra-embryonic tissues, e.g., placenta);
Multipotent (oligopotent) stem cells, which are able to differentiate into some types of cells having near properties (e.g., lymphoid and myeloid cells that take part in hematogenesis); and
Unipotent stem cells (in other word precursor cells, blast cells), which are specialized in repeated production of single-type cells-descendants (myelosatellitocytes that participate in formation of skeletal and muscular tissues).

Multipotent and unipotent stem cells designate quite often by common generic term ‘progenitor cells’.

Pluripotent and progenitor cells are presented within all live postnatal organisms as an internal resource for regenerative processes, but their part in aggregate cell mass is the lesser, the more is age of an organism.

At seventies of XX century cell therapy had arisen. It was considered as a transplantology partition that is based on use of suspensions of allogenic living stem cells, which can be obtained from corps of human embryos having gestational age no more than 12 weeks. It was established that such cells no contain HLA (human leukocyte antigens) and therefore have practically unlimited histocompatibility and are able to take root after injection into recipients' organisms, to proliferate and to fulfil necessary functions.

Initially the cell therapy was used only as alternative of bone marrow transplantation. For example, injection of native liver cells of a human embryo having gestational age seven weeks had allowed at firstly restoration of hemopoiesis for woman suffering from aplastic anemia [12].

Further, injection of fetal stem cells revealed positive results for treatment of primary and secondary myelosuppressive states [13].

J. I. Touraine had revealed possibility of the cell therapy in cases of serious combined immunodeficiencies that are caused by genetic defects. It was appeared that injections of genetically healthy pools of fetal hematopoietic stem cells into child organism (especially in case of early interference right up to antenatal period) allows compensating such immunodeficiencies [14].

However, long-term effects of use even histocompatible allogenic stem cells are not studied in detail until now. Moreover, obtaining of stem cells from abortive human embryos provoked ab origin deontological objections and has prohibited in many countries. Therefore, autologous stem cells use more often at present. Particularly, they can be separated in births from placenta, umbilical cord and amniotic fluid and then frosted for the purpose of long-term storage at liquid nitrogen temperature in stem cells cryobanks as a personal reserve of each concrete human.

It is obvious that this method of the cell therapy is very expensive, laborious and unsuitable for mass application. Moreover, many depositors of said cryobanks no require use of stored own stem cells during all life that makes storage expenses ineffective.

Way out of this situation was found in use of a pool of autologous pluripotent stem cells that can be extracted from some tissue of a postnatal organism, extra-corporally proliferated using suitable feeding formula and then returned into the same organism. Particularly, a dental pulp of milk teeth can serve as source of said stem cells for younger children [15]. It is understandable that other human tissues (e.g., adipose tissue of belly) can use for stem cells extraction depending on a patient's disease, age and level of health.

Naturally, a part of primarily extracted or earlier grown autologous pluripotent stem cells can be placed into a cryobank, if any patient needs multiple cell therapy acts, and then can be used, as necessity arises, for extracorporal incubation of required cell numbers.

At present preferably autologous pluripotent stem cells are successfully used for treatment of more than 100 diseases, for some of which the cell therapy is sole effective method. Moreover, injections of stem cells serve more often as important addition to surgical treatment or to pharmacological therapy. Accordingly, demand on stem cells grows in leaps and bounds. It can seem that this demand can be satisfied by extracorporal reprogramming of mature somatic cells into so called induced pluripotent stem cells (further iPSC).

Thereto J. Gurdon had placed into enucleated amphibian oocytes nuclei of somatic cells that give rise to new organisms [16]. Ipso facto it was revealed that genomes of even completely specialized cells remain genetically totipotent and can maintain development of whole organisms.

Later S. Yamanaka had ascertained that reprogramming can be also extra-corporally exercised for the purpose of iPSCs generation from a pool mice somatic cells using a cocktail of genetic factors Oct4, Sox2, Klf4 and c-Myc [17].

J. J. Gurdon and S. Yamanaka had been awarded for discovery of somatic cells reprogramming by Nobel Prize in 2012 [18].

However, extracorporal reprogramming of somatic cells into stem cells had not gone out scientific laboratories. Really, formation of totipotent stem cells by single-piece transplantation of somatic cells' nuclei into enucleated oocytes is reasonable as method of cloning that realizes until now only in scientific experiments, while reprogramming by transfer of allogenic genetic material into somatic cells (especially transcription factors such as retroviral or lentiviral vectors, which are able to induce transformation of postmitotic mature cells into pluripotent state) is characterized by extremely low productivity of labor, instability of composition and properties of made products and dearness of theirs [19].

This state of things cannot amend:
neither by extracorporal (i.e., only in vitro) non-viral gene transfection under influence of ultrasound having intensity no less than 0.05 W/cm² that leads alteration of their functions and, respectively, expression [19], nor by also extracorporal activation of mesenchymal stem cells proliferation under the influence of ultrasound with very high intensity 13.5 and 22.5 W/cm² [20];
neither by extracorporal (i.e., only in vitro) non-viral gene transfection under influence of ultrasound having intensity no less than 0.05 W/cm² that leads alteration of their functions and, respectively, expression [19], nor by also extracorporal activation of mesenchymal stem cells proliferation under the influence of ultrasound with very high intensity 13.5 and 22.5 W/cm² [20].

However, it is well-known from medical history that a way from laboratory experiments in vitro to clinical intervention into any human body in corpore is very long. Particularly, persons skilled in the medical art know that ultrasound is able to heat biological tissues the faster and stronger, the higher is its intensity, the deeper it penetrates into organism (and, therefore, the more complicated is heat removal) and the longer is exposition [21].

Therefore, creation of simple suitable and effective technology for intracorporal generation of local pools of pluripotent stem cells remains an actual problem.

As it occurs sometimes, an approach to acquisition of a required solution can be found off the well-trodden roads. In our case a hint had been obtained from such new branch of science as ‘mechanobiology’. It is aimed to study control over cells properties and tissues vital functions under influence of mechanical forces. Particularly, the scientific journal ‘Nature’ publishes systematically articles dedicated to this theme [22].

Specialists in mechanobiology had established—

That biological tissues can consider as media, in which interdependent cells can generate and perceive at the same time electrical, chemical and signals in the form of compression—distension (and, in other words, living cells can interchange information at mechanical level and perceive external force signals);

That mechanical impact can invoke rapid mitosis of at least epithelial cells;

Those nuclei of living somatic cells are biological analogs of strain sensors;

That low-powered force signals have an influence on processes within cytoplasm's and cells' nuclei and, respectively, can enhance stem cells proliferation;

That such force signals are able to reprogram mature specialized somatic cells at least into progenitor stem cells.

Particularly, now so-called “LIPUS” (i.e., “Low Intensity Pulsed Ultra Sound”) is using in therapy especially for the purpose of accelerated healing of wounds and bone fractures. Typically, this LIPUS is amplitude-modulated narrowband ultrasound having average intensity up to 200 mW/cm² (but preferably about 30 mW/cm²), fixed frequency (usually 1.5 MHz and rarer up to 10 MHz), and duty cycle 0.2 [23-27].

Intensity of ultrasound 30 mW/cm² is considerably less than value allowed for medical purposes. Therefore, such LIPUS cannot cause injury to an organism even during long exposure, but shows local therapeutic or cosmetic action. The clinical use of LIPUS signals for the treatment of wounds and bone fractures is authorized by Food and Drug Administration (USA).

Experimental studies of the therapeutic effect of narrowband LIPUS carried out on cell cultures or animals, and preclinical studies have shown the potential for widespread use of this therapeutic factor in the clinic. For example, it is shown that LIPUS:
significantly improves the condition of patients with chronic myocardial ischemia;
improves conditions after acute heart attacks;
prevents muscle atrophy induced by diabetes;
reduces cognitive dysfunction of the brain when modeling dementia and Alzheimer's disease;
inhibits the proliferation of breast cancer cells and osteosarcoma;
enhances the proliferation of osteoblasts and fibroblasts;
strengthens cellular and local immunity;
accelerates the regeneration of peripheral nerves;
reduces and prevents cerebral ischemia and vascular damage during an experimental stroke;
forms new blood vessels and stimulates cellular regeneration in the brain;
reduces erectile dysfunction;
accelerates osseointegration of orthopedic implants;
partially restores joints;
induces apoptosis of hepatocellular carcinoma cells.

The list of the above diseases and pathological conditions in which LIPUS is effective, almost completely coincides with diseases in which stem cell therapy is effective [28].

A fundamentally important advantage of low-intensity ultrasound therapy is that the process of cell treatment involves one's own, and not transplanted foreign or chemically reprogrammed stem cells containing foreign reprogramming factors in genes. Therefore, mechanisms of tumor development are excluded due to the negative influence of foreign structures on the fate of cells (and the body). There is also no immune response to the rejection of the “alien” due to the fact that their own stem cells are activated.

Despite the attractiveness of LIPUS as a unique therapeutic factor, and for more than 30 years of research, so far it has been used to a limited extent, for example, to accelerate the healing of bones and soft tissues, as well as to accelerate the implantation of implants. The main reason for the limited use of LIPUS is the low efficiency of diseases treatment.

It became clear that new signals were needed for a more accelerated course of reparative processes at all levels of the cell organization—extra- and intracellular matrix, membranes and internal structures of cells, nuclei and DNA.

William Tyler radically improved the LIPUS method of treating many diseases, mainly neurological. Starting from the year 2010 and up to the present time [30-57], many modes of operation and parameters of LIPUS ultrasound have been proposed, firstly in the frequency range from 0.02 to 1.0 MHz, and later to 100 MHz, and Ispta intensity from 0.0001 to about 900 mW/cm². Different therapeutic narrowband waveforms are applied, such as harmonic signals, and/or any repetitive impulses or their combinations, to obtain the stimulus waveforms containing one or many ultrasonic frequencies. Periodically repeating waveforms as a single or plurality pulses are also possible. Each impulse includes from 1 to 50,000 acoustic cycles, repeating with frequencies from 0.001 to 100 kHz, that is, it is emitted in the form of a comb spectrum.

In other words, W. Tyler proposes a low-intensity ultrasound therapy using one modulated harmonic signal or using multiple harmonic signals or using multiple comb signals, or combinations thereof. The finite set of harmonic W. Tyler signals is generally narrowband, and, therefore, fractional bandwidth η<0.2.

A physical reason for the low clinical effectiveness of LIPUS may be the discrepancy between the parameters of narrow-band ultrasound signals intended to affect cells and cell genomes and those signals that are similar to the intrinsic micromechanical signals of cell nuclei, cells, cell ensembles and tissues. In addition, not only functional and stem cells, but also cells of the immune system, extra-inter- and intracellular scaffolds and communication structures, as well as many other local and blood-borne participants in regenerative processes take part in regenerative processes. Therefore, ultrasonic/mechanical action at a single frequency or even at many frequencies may not provide interaction at many levels of organization of biological tissue activity.

It is known that the amount of information (or energy) that can be transmitted from a source to a receiver is proportional to the frequency bandwidth of the signal and the dynamic range of the signal. When passing from several or from a finite set of narrow-band harmonic signals to a continuous band, that is a very large number of frequencies, it is possible to transmit a much larger amount of information (or energy) with better noise immunity. For this reason, at the end of the 70s of the last century, ultra-wideband technology for communications and radars appeared, which dramatically improved the technical characteristics of the devices [58-59].

In 2011 Mahfouz M. et al. described a surgical navigation ultra-wideband electromagnetic system for orthopedics [60].

To date, the development of ultra-wideband electromagnetic systems for imaging and diagnostics of internal organs is being completed [61].

Ultra-wideband medical devices began to be developed since 1981 by the author of the present invention, A. Marchenko, after the creation by him and his team broadband (later ultra-wideband) ultrasonic multimode transducers with a transducing efficiency comparable to mono frequency ones (Marchenko A. et al, [62-65]).

On their basis, ultra-wideband therapeutic and cosmetology ultrasonic devices were created: RU2066215C1, Marchenko A. et al., 1996 and RU2058167C1, Marchenko A. et al., 1996 [67].

In 2014, based on the aforementioned multimode transducers, Tereschenko N. et al. patented (Patent UA 91162) an ultra-wideband ultrasound therapy system [68]. Known for all of us as ultra-wideband therapeutic devices using a band of ultrasonic frequencies from about 1.0 to about 5.0 MHz in the form of frequency-varying harmonic signals or stochastic continuously changing in frequency signals. The ultrasound intensity from 0.1 to 0.6 W/cm² was chosen.

Limited clinical trials carried out jointly with the author of ultra-wideband devices Marchenko A. at the Kyiv Otorhinolaryngology Institute showed significantly better results in the treatment of chronic tonsillitis compared to classical therapeutic ultrasound devices [69-70].

Developing the technical solutions of A. Marchenko and mentioning his invention according to patent RU 2066215 as an analogue, the authors NARAIKIN O. S. and SAVRASOV G. V. proposed a device and method for ultrasonic therapy of biological tissues [71]. In this device, the impact is carried out by ultrasonic fields having separate frequencies, approximately corresponding to the natural frequencies of mechanical vibrations of the cell membrane (that is, resonant frequencies). The device contains a generator of ultrasonic vibrations, an acoustic unit and an ultrasonic tool, which is a replaceable working body having a working end, as well as a fluid supply system.

In the invention of J. W. Huckle et al. US2003/053849 proposed a therapeutic effect of ultrasonic low-intensity narrow-band amplitude-modulated signals with frequencies of 1.0 and 1.5 MHz. The purpose of the device is the treatment of the connective tissues of the body.

Also known systems, devices and methods of using of therapeutic ultrasound, proposed by Shields Donald J. in US patent application US2007249938 (A1) [73]. The ultrasound therapeutic device according to this invention includes a signal generator, one or more transducers, one or more sensors, and a controller. The generator generates one or more narrowband pulse signals with frequencies of 1-2.5 MHz. Signal intensity mW/cm². In some embodiments of the invention, the signal generator is configured to generate a first ultrasonic signal having at least a first harmonic waveform segment and a second harmonic waveform segment of an ultrasonic waveform different from the first waveform segment.

These known low-intensity ultrasonic devices and methods of treating many diseases by changing cellular activity may be based on the use of one or many individual frequencies, which affect separate resonating structures of tissues and cells.

### REFERENCES CITED ABOVE

21. Maximov A. Der Lymphozyt als gemeinsame Stammzelle der verschiedenen Blutelemente in der embryonalen Entwicklung and im postfetalen Leben der Säugetiere/Folia Haematologica.—1909, S. 125-134.
22. Kelemen E. Second J. Gematol., 1973, v. 10, No. 4, pp. 305-308.
23. Izzi T., Polehi O. et al. Fetal liver transplantation, Alan R. Liss, 1985, pp. 237-249.
24. Touraine J. I. Transplantation Proceedings, 1993, v. 25, No. 1, pp. 1012-1013.
25. U.S. Pat. No. 9,175,264 Songtao Shi et al. Postnatal Stem Cells and Uses Thereof. 2015.
26. Gurdon J. B. The developmental capacity of nuclei taken from intestinal epithelium cells of feeding tadpoles/J Embryol. Exp. Morphol.—1962, V. 10, No 4, pp. 622-640.
27. Takahashi, K.; Yamanaka, S. Induction of Pluripotent Stem Cells from Mouse Embryonic and Adult Fibroblast Cultures by Defined Factors/Cell.—2006, V. 126, No 4, pp. 663-676.
28. Shinya Yamanaka, Sir John Gurdon. Nobel Prize in Medicine 2012.
29. Newman C. M., Lawrie A., Brisken A. F. Cumberband D. C. Ultrasound gene therapy: on the road from concept to realty. Echocardiography, 2001, pp. 329-347.
30. Jia-Ling Rua, Yak-Nam Wang, Lawrence A, Crum. Proceedings of 2010 IEEE International Ultrasonic Symposium.
31. C. R. Hill, J. C. Bamber, G. R. Tear Haar, Physical Principles of Medical Ultrasonics. 2nd Edition, 2004.
32. Website https://www(dot)nature(dot)com) nature cell biology.
33. U.S. Pat. No. 4,530,360. Duarte L. R. Method for healing bone fractures with ultrasound, 1985.
34. U.S. Pat. No. 5,904,659. Duarte L R et al. Ultrasonic treatment of wounds. 1999.
35. U.S. Pat. No. 6,231,528. Kaufman J. J. et al. Ultrasonic and growth factor bone therapy: apparatus and method. 2001.
36. Website www(dot)electrotherapy(dot)org.
37. Booklet: Low Intensity Pulsed Ultrasound, Tim Watson, 2012.
38. Ranjeet Singh Machla. Stem Cells Application in Regenerative Medicine and Disease Therapeutics. International Journal of Cell Biology. Volume 2016, Article ID 6940283, 24 pages.
39. WO 2010/009141A1. Tyler W. Method and Devices for Modulating Cellular Activity Using Ultrasound.
40. US2011/0178441. Tyler W. Methods and Devices for Modulating Cellular Activity Using Ultrasound
41. US2012/0296241. Tyler W. Ultrasound Neuromodulation for Treatment of Autism Spectrum Disorder and Alzheimer's Disease and Other Dementias
42. US2012/0289869. Tyler W. Devices and Methods for Modulating Brain Activity
43. US20120283604. Tyler W. Ultrasound Neuromodulation Treatment of Movement Disorders, Including Motor Tremor, Tourette's Syndrome, and Epilepsy
44. US2012/0245493. Tyler W. Ultrasound Neuromodulation Treatment of Addiction
45. US2012/0232433. Tyler W. Ultrasound Neuromodulation Treatment of Tinnitus
46. US2012/0226091. Tyler W. Ultrasound Neuromodulation Treatment of Pain
47. US2012/0220812. Tyler W. Ultrasound Neuromodulation for Stroke Mitigation and Rehabilitation
48. US2012/0083719. Tyler W. Ultrasound-Intersecting Beams for Deep-Brain Neuromodulation
49. US2013/0281890. Tyler W. Neuromodulation Devices and Methods
50. US2013/0261506. Tyler W. Ultrasound Neuromodulation Treatment of Post-Traumatic Stress Syndrome
51. US20130184728. Tyler W. Ultrasound Neuromodulation for Diagnosis and Other-Modality Preplanning
52. US2013/0144192. Tyler W. Ultrasound Neuromodulation Treatment of Anxiety (Including Panic Attacks) and Obsessive-Compulsive Disorder
53. US2013/0096469. Tyler W. Ultrasound Neuromodulation Treatment of Obesity and Eating Disorders
54. US2013/0079682. Tyler W. Ultrasound-Neuromodulation Techniques for Control of Permeability of The Blood-Brain Barrier
55. US2014/0364774. Tyler W. Ultrasound Neuromodulation for Clinical Effects
56. US2014/0194726. Tyler W. Ultrasound Neuromodulation for Cognitive Enhancement
57. US2014/0107533. Tyler W. Ultrasound Neuromodulation Treatment of Attention Deficit Hyperactivity Disorder
58. US2014/0094720. Tyler W. Methods and Devices for Modulating Cellular Activity Using Ultrasound
59. US2014/0094719. Tyler W. Ultrasound Neuromodulation Treatment of Schizophrenia
60. US2014/0088462. Tyler W. Ultrasound Neuromodulation Treatment of Gastrointestinal Motility Disorders
61. US2015/0025422. Tyler W. Methods and Devices for Modulating Cellular Activity Using Ultrasound
62. US2016/0303402. Tyler W. Methods and Devices for Modulating Cellular Activity Using Ultrasound
63. US2016/0220850. Tyler W. Devices and Methods for Modulating Brain Activity
64. US 2016/0001096. Tyler W. Devices and Methods for Optimized Neuromodulation and Their Application
65. US2017/0246481. Tyler W. Devices and Methods for Optimized Neuromodulation and their Application.
66. US2019/0105517A1. Tyler W. Devices and Methods for Modulation Brain Activity.
67. US 2020/0246641A1. Tyler W. Methods and Devices for Modulation Cellular Activity Using Ultrasound.
68. Terence W. Barett. History of Ultra-WideBand (UWB) Radar & Communications. Pioneers and Innovators.—Progress in Electromagnetics. Symposium 20 Jul. 2000.
69. Chernogor I. F. et al. Ultra-wideband signals: theory, simulation and digital processing/In Proceeding. Ultra-wideband and Ultrashort Impulse Signals.—Sevastopol, 2006.—pp 32-37.
70. Mahfouz M. et al. The Future of Ultra-Wideband Systems in Medicine: Orthopedic Surgical Navigation. In Book: Novel Application of UWB Technologies. 2011. DOI: 10.5772/2050/.
71. Discussion. Ultra-Wideband (UWB). A New Definition of the Future. https://litum(dot)com.2021.
72. Copyright certificate No. 1323141 (3267992) dated 21 Apr. 1981. Marchenko A. T., Bakaj E. A. Ultrasonic Broadband Transducer.
73. Ostrovsky I., Marchenko A. Variable Thickness Piezoelectric Transducers for Medical Applications./35th Annual of the ALUM, Georgia, USA, 1991, Abstract #4012.
74. Ostrovsky I., Marchenko A. Ultrasonic Instrument for Information Action on Biological Objects and Tissues./J. Ultrasound Med.—1992.—V.11, #3.—p. S57.
75. Ostrovsky I. V., Zhabitenko M. K., Marchenko O T. Broadband ultrasound emitters. Ukrainian Journal of Medical engineering and Technology 1994, N21, p. 29-35.
76. RU 2066215 C1. Marchenko A. T., Ostrovsky I. V. Ultrasonic Therapeutic Apparatus. Sep. 10, 1996.
77. RU 2058167 C1. Marchenko A. T., Ostrovsky I. V. Device for Ultrasound Therapy and Cosmetology. Apr. 20, 1996.
78. UA 91162. Tereschenko N. et al. Ultrasonic Automated Therapeutic System. 2014.
79. Zabolotny D. I., Karas A. F., Marchenko A. T. and other Clinical and morphofunctional characteristics of the tonsils in patients with chronic tonsillitis after exposure to low temperatures and broadband ultrasound./YII Congress of otorhinolaryngologists of the Ukrainian SSR 4-6 Oct. 1989, Odessa./Kiev, 1989.—p. 304-305.
80. Zabolotny D. I, Marchenko A. T. Prospects for the use of broadband ultrasound in otolaryngology./YI Congress of otorhinolaryngologists of the RSFSR. Abstracts. Dokl. 19-21 Sep. 1990-Orenburg, 1990.—p. 86-88.
81. WO0204074 (A1). Naraikin 0. S. and Savrasov G. V. Method and devise for therapy of biological tissues using an ultrasound field.
82. US2003/053849. J. W. Huckle et al. Metod and Apparatus for Connective Tissue Treatment.
83. US2007249938 (A1). Shields Donald. Systems, Devices, and Methods Employing Therapeutic Ultrasound of Living Tissues.
84. US2013226000 (A1). A Kim Bae Hyung et al., Apparatus and Method for Generating Ultrasonic Image.
85. Ingber D. E. The architecture of life/Scientific American. 19984 278 (1): 48-57.
86. Levin Stephen (2015). Tensegrity. The new biomechanics. Oxford Textbook of Musculoskeletal Medicine. Oxford University Pres, pp 155-160. ISBN 978-0-19-967410-7.
87. Lomakin A. J. et al. The nucleus acts as a ruler tailoring cell responses to spatial constraints. Science 2020 Oct. 16; 370 (6514): eaba 2894, doi: 10.1126/science.aba2894.
88. Joseph T. Long and Jan Lammerding. Nuclear Deformation Lets Cells Gauge Their Physical Confinement/Development cell 56, Jan. 25, 2021. Elsevier Inc.
89. Steven Boeunaems et al. Phase separation in Biology and Disease; Current Perspectives and Open Questions/J Mol Biol. 2023 Mar. 1; 435 (5), 167971.
90. DE102011115906A1. Kruglikov Ilja. System for Generating Ultrasonic Waves and Procedures for Configuring an Ultrasonic System. 2013.

Biological tissues, cells and intracellular structures that include the generation of electrical and chemical signals, as well as the generation and receiving of mechanical signals. The intensity of such signals is very small, so we call them micromechanical. It was revealed that weak information signals by cells propagate among intense thermal micromechanical noise.

Reliable reception and transmission of signals under such conditions may be possible only when encoding and subsequent decoding by cells of ultra-wideband signals.

Thus, in biological media, there should be multilevel ultra-wideband sources of coded micromechanical information and ultra-wideband decoding receivers, further transmitting the received information to biological process control systems at different levels of cellular and tissue organization.

It has been found that ultra-wideband systems, that is, systems having a very large number of resonant frequencies of mechanical vibrations, may be able to have a conversion efficiency comparable to narrowband resonators [63-65]. This is possible for multimode oscillatory systems, the modes of which overlap, and is implemented in many acoustic musical instruments (violin, guitar, plano).

Reliable separation of wideband signals from noise may not require high quality factor (selectivity) of signal transmitters and receivers or their tuning to a specific frequencies, since the information signal is formed from a huge number of components inside the ultra-wide band. Transmission and reception in ultra-wide frequency bands are characteristic of biological tissues and cells that oscillate in highly damping and noisy media.

All organisms have structures that allow them to generate, receive, recognize and respond to mechanical signals. Interaction biostructures with micromechanical signals occurs on:
tissue level—tissues, blood vessels, blood, extracellular environment, cells of the immune system, nerve fibers, Characteristic dimensions of tissues—fractions—units of millimeters;
cellular level—individual cells, cell membranes, intracellular matrix, organelles, nuclear shell, intranuclear matrix and DNA. Their characteristic dimensions are units—hundreds of micrometers.

Any living system consists of a spatially organized three-dimensional ensemble of cells, extra- and intra-cellular structures similar to each other, the spatial shape of which is maintained due to a three-dimensional framework of stretched threads of biomolecules, that is, “tensegrity” (tensional integrity) [75-76].

The same three-dimensional resonance structures “tensegrity” form extracellular, intracellular and intranuclear matrices. All living organisms use “tensegrity” to stabilize their form, as well as to detect and transmit micromechanical signals to multiple levels of the cellular hierarchy, including DNA. This is part of the structures that make it possible to generate, recognize and respond to mechanical forces.

In our opinion, the generators of control signals may include the totality of all possible elastic (viscoelastic) structures of tissues and cells, which fluctuate in ultra-wide bands at each of the possible levels of cellular organization, due to thermal vibrations of structures and elements. In particular, tissue structures oscillate with frequencies of 1-10 MHz, respectively, the dimensions of the “tensegrity” elements of tissues are units-fractions of millimeters. Cell membranes, their intracellular matrix and other elements of the micromechanical environment fluctuate in the frequency bands of 10-100 MHz and, accordingly, have characteristic dimensions of tenths-hundredths of millimeters. The intranuclear matrix and DNA strands have characteristic dimensions of the order of 1-10 μm and resonant ultra-wide frequency bands in the frequency range of hundreds of MHz. It is clear that the frequency bands of the lowest level (a few megahertz) may also slightly affect the highest level of cellular activity, for example, gene expression and cell transformation, sequentially ascending to DNA along the transduction structures conducting ultra-wideband micromechanical oscillation spectra.

According to this concept, each biological structure, for example, a cell, constantly oscillates on a multitude of adjacent (merging) frequencies, that is, on ultra-wide bands, and transmits this “oscillation symphony” to neighboring cells of the same (in the general case) that oscillate on the same ultra-wide bands.

Because there are many similar cells, the entire ensemble of cells may sound in harmony. When functioning is disturbed at any hierarchical level of vital activity of cellular structures, beats may occur between healthy and diseased cells-signals of falseness, illness, that is, there may be regulatory feedback micro-signals. Rising along the pathways of mechano-transduction, feedback signals may turn on the necessary responses that regulate the vital activity of biological cells and tissues.

Even if we do not know the corrective signals that control the functions of tissues and cells, we may be able to significantly enhance and accelerate the regenerative processes both in normal conditions (reparation, rejuvenation, treatment) and in pathologies-wounds, traumas, inflammations, scars, burns, ischemia areas (regeneration).

To speed up and increase the efficiency of reparative and regenerative processes, we propose to apply ultra-wideband micromechanical signals to the pathological site and surrounding healthy tissues, similar to those that are exchanged between cells and tissues. This means that ultra-wideband signals of very low intensity with a continuous emission spectrum within the frequency range of 1-250 MHz may be needed. Depending on the desired level of the exposure hierarchy, i.e. tissue, cellular, intracellular or intranuclear, it may be necessary to select the correct ultra-wideband range (as shown above), or radiation in all ranges simultaneously. Such signals may provide additional micromechanical energy to all mechanosensitive levels of the tissue and cellular hierarchy, supplementing the energy of thermal vibrations. As a result, the reliability of the transmission of information signals within and between cells may increase. The number of cells simultaneously interacting with each other may also increase, that is, their coordination number and the number of ongoing processes per unit time may increase many times over. Thus, we can expect multiple acceleration and intensification of reparative and regenerative processes. This is how natural reparative processes proceed normally.

During regenerative processes, more complex processes of healing and replacement of lost or pathological tissue areas may occur. Due to micromechanical reprogramming, stem cells may multiply intensively, that is, proliferative processes may be induced. Due to the initiation of reprogramming some somatic cells may be transformed into progenitor and pluripotent stem cells. Deviant or diseased cells may undergo death on command-apoptosis. There is a cleansing of the pathology zone with its further filling with multiplied cells. In healthy tissues surrounding the area of pathology, due to the homing effect and the reaction of the immune system, not only stem cells attracted from the periphery accumulate, but also cells of acquired immunity, including lymphocytes, and innate immunity-macrophages. The joint functioning of the complex of restoring stem and immune cells leads to the filling of the pathological zone with tissue-specific cells, with further germination of microvessels, vessels and innervation of the filled volume, as well as to the restoration of the outer integument of the body.

Relatively recently, it became known [77-78] that the shells of cell nuclei and DNA molecules are not only mechanosensitive elements, but also sense space and environment. The nucleus, in addition to its genetic functions, directly senses the physical environment of the cell and can control the movement of immunocompetent cells to the area of pathology. Under the action of ultra-wideband micromechanical signals, cell contractility may increase and the velocity of migration of lymphocytes and macrophages to the areas of regeneration and repair may increase and, consequently, the therapeutic effect may improve.

In some embodiments of the present invention, ultra-wideband devices for UWB micromechanical action with frequency bursts of micro-energy having continuous constant, increasing or amplitude modulated power frequency density level is exerted on many levels of cellular, extracellular and intracellular organization of tissues and cells to enhance and accelerate the natural processes of repair and regeneration in them.

To implement such an impact, by authors of this invention described herein the device for ultra-wideband (UWB) micromechanical frequency multilevel regenerative burst-therapy, according to an exemplary embodiment of the invention.

The device contains at least one main generator of ultra-wideband (UWB) electrical spectrum, at least one generator of correcting UWB electrical spectrum, bandpass filters unit, UWB spectrum shape corrector, variable gain amplifier, UWB micromechanical transducer of electrical spectrum into micromechanical treating frequency bursts (UMB transducer), UWB protector, executive microcontroller, central processor unit and microprocessor for monitoring the presence of contact of the transducer with the patient's body.

The at least one main generator of UWB electrical spectra is connected to the first input of the UWB spectrum shape corrector through a unit of bandpass filters. The at least one correcting generator of UWB electrical spectrum is connected through a unit of bandpass filters to the second input of the specified UWB spectrum shape corrector, and the output of this corrector is connected to the input of the UWB gain amplifier, and the output of the last is connected to the electrodes of the UMB transducer, acoustically attached to the UWB protector, in turn, acoustically connected through the contact medium to the surface of the patient's body.

All these elements of the device are designed to form electrical spectra useful for the treatment using UWB, their subsequent conversion into UWB micromechanical frequency bursts, the shapes of the spectra of which may be different from those directed downwards, as well as to introduce these frequency bursts through the surface of the patient's body into the treatment area and neighboring healthy tissues in order to influence all cellular structures with micromechanical UWB frequency bursts and cause their multiple own oscillations, thereby forming micromechanical therapeutic “images” identical to natural images that occur during the life of cells and tissues.

Besides the device also contains an executive microcontroller, central processing unit (CPU) and unit a microsignal monitoring of acoustical contact. The microcontroller is connected to the inputs of the main and corrective generators, and this controller is also a programmer and an indicator of the state of the claimed device, which in turn is controlled by the central processing unit (CPU).

According to an exemplary embodiment of the present device the mentioned UWB spectrum generators, together with band-pass filters unit, the UWB spectrum shape corrector and the UMB transducer form treating UWB micromechanical frequency bursts having at least one continuous UWB frequency range inside the band of 1-250 MHz, which has spectrum shapes different from downward, while the mentioned band consist of one or more ultra-wideband frequency ranges, including 1-3, 3-10, 10-30, 30-100, 100-250 MHz.

The proposed device contains two or more UWB pulse generators, which, together with the band-pass filter unit, the UWB spectrum shape corrector and the UMB micromechanical frequency transducer, create therapeutic UWB micromechanical frequency bursts, forming a continuous band and this UWB band (spectrum) may consist of several UWB ranges.

The executive microcontroller of the main, corrective and other generators also performs the functions of an indicator of the current state of this device and is connected to a detector of the presence and degree of acoustic contact between the UMB transducer and the patient's skin. The micro signal contact detector includes: a) a sensor for determining the parameters of the contact, formed by the front and lateral electrodes of the micromechanical volumetric UWB transducer and the sensing part of the transducer volume located between them; b) synchronous amplifier, c) hoarder of control signals, d) setter of values of parameters of signals of presence and degree of contact, e) signal processor of the contact detector.

The executive microcontroller is connected to at least two UWB pulse signal generators main and correcting spectra, and to a narrowband low frequency LF pilot signal generator, to synchronous amplifier and to a contact signal microprocessor.

The device contains a side electrode placed on the back surface of the UMB transducer, which is connected in series with the first input of the synchronous amplifier, the hoarder, the contact signal microprocessor, and with the second input of the executive microcontroller. The third output of the mentioned microcontroller is connected to the second input of the synchronous amplifier. The signal from the third output of the microcontroller is delayed relative to the signal of the first output by more than on 0.2 μs.

According to an embodiment of the present device it consists of a master part—a remote controller and an executive part—a mobile applicator for UWB non-invasive micromechanical frequency burst regenerative therapy with the electronics unit placed inside or outside the mobile applicator. Herewith the master part is CPU—central processing unit, connected to the executive microprocessor via Bluetooth, Wi-Fi or a radio module. CPU is an autonomous unit, performed as a mobile phone, tablet, mobile or desktop computer. The executive part also contains a Bluetooth or Wi-Fi or a radio module and a rechargeable power source with a charging microcontroller.

According to an embodiment of the present device, the various amplitude distribution of the frequency power density and the shape of signals of spectrum of at least one main generator is selected from a variety of main spectra, including for example shock-wave, stress-wave, and from rectangular, triangular, trapezoidal form of spectra, or their differentials, or their combinations. Wherein the correcting spectra of at least one signal is selected from a variety of signals, for example, from the differentials of the main pulse signals, as well as low-cycle sinusoidal signal including, a Gaussian monocycle, a low-cycle sinusoid, a Sinus Cardinalis (Sinc signal), or combinations thereof.

According to an embodiment of the present device the applicator is designed to introduce treating ultra-wideband micromechanical frequency bursts into the treatment area through the surface of the skin. In addition, the applicator may be designed to introduce ultra-wideband micromechanical bursts into conjunctival surface of the eye, as well as natural body entity openings, such as nasal cavity, oral cavity, esophagus, rectum, vagina.

Another object of the invention, in some embodiments, is the method of operation of the device for UWB micromechanical regenerative frequency burst therapy. This method includes a series of successive steps. Firstly, it is generating at least one pair of complementary electrical UWB signals consisting of the main and corrective electrical signals with different amplitude distributions of the power frequency density, designed for at least one ultra-wideband frequency range of therapeutic signals within the band 1-250 MHz. After that is carried out correction of the UWB spectra of the main and corrective electrical signals in at least one frequency range using band-pass filters and spectrum corrector, and summation of the spectra to obtain spectra with a constant, increasing, an arbitrary shape of the spectra and the various amplitude distribution of frequency power density. Then is carried out conversion of UWB electrical spectra into treatment UWB micromechanical frequency bursts and delivery of treating UWB micromechanical frequency bursts through acoustically transparent UWB protector and UWB conductive medium to the body surface. Further performs non-invasive input non-invasive input and delivery of micromechanical UWB frequency bursts to the affected area and tissues adjacent to it. The burst field is optionally configured so that in order to cause a variety of possible own micromechanical oscillations of cells and tissues, thus forming UWB micromechanical therapeutic “images” similar to natural images that arise in the process of life, that is, cause the radiation of own micromechanical therapeutic signals in the entire ultra-wide frequency range, for example 1-250 MHz.

There is also possible to induce your own frequency micromechanical treatment responses in only one ultra-wide range or several ultra-wide ranges, for example: a) in the 1-5 MHz range-stimulation of regenerative processes in biological tissues, acceleration of growth of tissues, nerves and blood vessels, wound healing; b) in the 1-MHz range-excitation of the activity of the extracellular matrix, increased apoptosis, increased local immunity, increased proliferative and migration processes and the activity of stem cells existing in tissues; c) in the 10-30 MHz range-stimulation of protein synthesis, stimulation of stem cell programming, direct reprogramming of progenitor cells, stem cell reproduction, a general increase in local cellular activity and repair processes; and d) in the 30-100 MHz range—an increase in the activity of processes caused by deformations of the nuclear membranes, processes of epigenetic reprogramming, gene expression, transformation of somatic cells into stem cells.

Further it is possible transfer of treating energy of UWB micromechanical frequency bursts to a variety of mechano-sensitive biological structures, including cellular ensembles, extracellular and intercellular matrices, blood vessels, blood cells, nervous system, immune and stem cells, cytokines, chemokines and other biologically active factors, cell nuclei and DNA.

Generally, UWB micromechanical frequency burst-stimulation or modulation of regenerative processes at various levels of the aforementioned mechanosensitive tissue structure, optionally including reprogramming of some somatic cells into multipotent, progenitor and pluripotent stem cells and, consequently, acceleration and enhancement of natural regenerative processes.

In addition, in accordance with the proposed method, there may be performed transfer of treating energy of UWB micromechanical frequency bursts to a variety of mechano-sensitive biological structures, including cellular ensembles, extracellular and intercellular matrices, blood vessels, blood cells, nervous system, immune and stem cells, cytokines, chemokines and other biologically active factors, cell nuclei and DNA. Further there may be performed UWB micromechanical frequency burst-stimulation or modulation of regenerative processes at various levels of the aforementioned mechanosensitive tissue structure, including reprogramming of some somatic cells into multipotent, progenitor and pluripotent stem cells and, consequently, acceleration and enhancement of natural regenerative processes. And finally carried out UWB micromechanical frequency burst stimulation of the processes of accumulation of pluripotent, multipotent, progenitor stem cells and immune cells in and around the treatment area by repeating therapeutic micromechanical frequency bursts act for many, up to 60 days [75-76].

According to one exemplary proposed method, at least one the first therapeutic UWB micromechanical frequency burst, formed by the first main and complementary to it first correcting spectrum, has the first frequency range with the first shape of the spectrum, which may have a stressful effect on the tissues and on apoptosis processes in by changing the state of the extracellular matrix in the affected area and surrounding healthy tissues.

However, at least one more, the second therapeutic UWB micromechanical frequency burst, formed by the second main and complementary second correcting spectra, has at least a second frequency range with a second shape of the spectrum.

The proposed method stimulates in the somatic cells of the treatment area and surrounding healthy tissues the processes of reprogramming, direct reprogramming, differentiation, proliferation, tissue formation and replacement of diseased or damaged cells by healthy ones.

According to an exemplary embodiment of the invention, the first therapeutic UWB micromechanical frequency bursts formed in the first frequency range of 1-3, 3-10 MHz, and have in the treatment area an intensity averaged over frequency, space and time in the range of about 1-500 mW/cm².

The second treating UWB micro-mechanical frequency bursts are formed in frequency ranges of 10-30, 30-100 or 100-250 MHz, and have an intensity of frequency bursts averaged over the frequency of time and space on the body surface of 0.001-100 mW/cm² and in the treatment area of 0.0001-30 mW/cm².

Herewith a plurality of these first and second therapeutic UWB micromechanical frequency bursts form a common band of the therapeutic spectrum and are repeated during treatment in the frequency ranges 1-3, 3-10, 10-30 MHz with a frequency of 0.05-1 kHz, and in the frequency ranges of 30-100, 100-250 MHz with a frequency of 0.05-103 kHz.

According to this embodiment of the proposed invention, the UWB micromechanical frequency bursts, formed by this device may apply in regenerative medicine to treat diseases selected from the group, including: stroke, myocardial infarction, ischemia, spinal cord injury, degradation of the retina and optic nerve, glaucoma, diseases of the musculoskeletal system, diabetes, some types of cancer, atherosclerosis, varicose veins, post-traumatic, post-burn and postoperative wounds, rejuvenation of the skin and other structures of tissues and organs of the body.

Furthermore, the treating ultra-wideband micro-mechanical frequency bursts, formed by the device may be applicable in regenerative cosmetology to treat diseases and pathological skin conditions including signs of skin aging, scars, acne, wrinkles, melasma, swelling, edema.

According to an embodiment of the proposed invention, the shapes of spectra of the main generators are selected from a variety of spectra, including shock wave, stress wave, as well as from the spectra of rectangular, triangular, trapezoidal pulses or combinations thereof. The shapes of the spectra of corrective generators are selected from a variety of main signals spectra and their differentials, as well as from a Gauss monocycle, a low-cycle sinusoid, a cardinal sinusoid (Sinc signal), spectra of arbitrary waveforms, or combinations thereof. The sequence of therapeutic frequency bursts can be coherent, as well as incoherent.

According to another embodiment of the proposed invention the transducing of electrical spectra into micromechanical treating frequency bursts is performed by more than one UMB transducer. An embodiment of the invention is possible, in which the conversion of electrical spectra into micromechanical therapeutic frequency bursts is carried out by several ultra-wideband transducers, which are placed in a single housing, with the ability to move inside the latter in three planes (a system of “floating” transducers).

The treating UWB micro-mechanical frequency bursts from the transducer to the surface of the body are fed to the body surface through acoustically transparent and acoustically coupled elements, such as ultra-wideband protector, acoustically transparent contact layer or an extended contact medium, placed between the protector and the body surface.

According to an embodiment of the invention, treating ultra-wideband micro-mechanical frequency bursts are introduced into the treatment area through the conjunctival surface of the eye, as well as directly into the orifices of the body, such as the nasal cavity, oral cavity, esophagus, rectum, vagina.

The therapeutic impact on the pathological area and surrounding healthy tissues with ultra-wideband micromechanical frequency bursts is, for example, carried out daily for 5-40 minutes for 5-60 days.

The foregoing and other features of the present invention are more fully described below, whereas the following description detailing an illustrative embodiment of the invention, but also indicating some of the different directions in which the principles of the present invention may be employed.

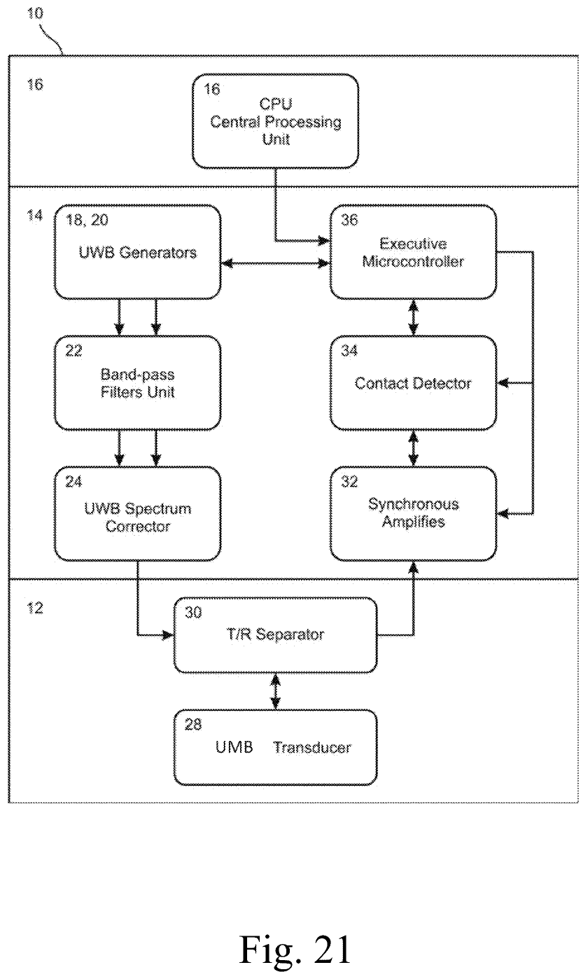

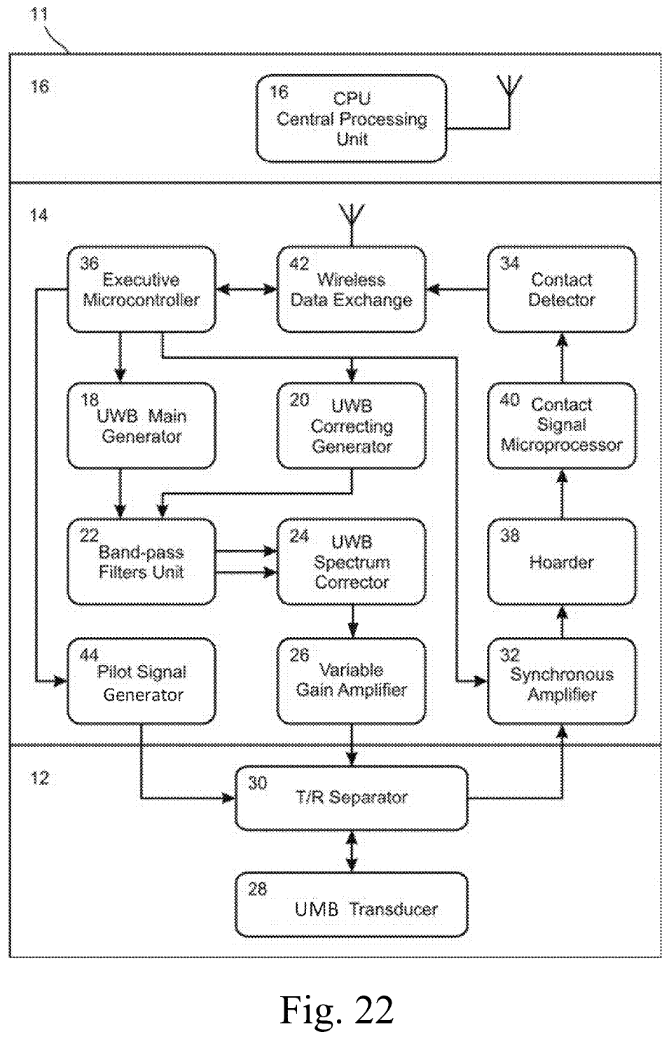

The ultra-wideband micromechanical devices 10 and 11 for regenerative frequency burst therapy consists of three main units: (see FIG. 21 and FIG. 22):
unit of electromechanical transducer 12;
electronics unit 14;
central processor unit (CPU) 16.

In the figure FIG. 21 shows a general structural block diagram of stationary device 10 for ultra-wideband micromechanical regenerative frequency burst therapy.

Electromechanical transducer unit 12 contains ultra-wideband transducer 28 or a set of piezoelectric transducers, which together provide operating frequency bands of micromechanical signals 1-10; 10-30; 30-100; 100-250 MHz or their combination. In the immediate vicinity of the piezoelectric transducers, transmission/receiver (T/R) separator 30 is placed, which separates the signals of generators 18, 20 and signals to synchronous amplifier 32. The overall control of the operation of the device is carried out by central processor unit (CPU) 16, which is connected to executive microcontroller 36.

Main signal generator 18 (see FIGS. 21 and 22), which is located in the electronics unit, generates ultra-wideband spectra in a frequency band specified at 6 dB. The signals from generator 18 are fed to the first input of band pass filter 22, which ensures the passage of the spectrum only in a given frequency band. At least one corrective signal generator 20 optionally generate spectrum to correct of the main generator spectrum. The pulses from the latter are fed to the second input of bandpass filter unit 22 and are limited in the required frequency band.

From the first and second outputs of the bandpass filter unit, the filtered main and corrective ultra-wideband pulses are fed to the first and second inputs of spectrum corrector 24. After the summation of two or more signals with different shape of spectra in the spectrum corrector, the spectra from the UWB spectrum corrector output through T/R separator 30 are fed to UMI burst transducer 28. The latter converts the electrical spectra into UMB frequency bursts.

Micromechanical frequency bursts from UMB transducer 28 through the gel contact layer come to the skin or other accessible areas of the patient's body and then reach the diseased or altered area and surrounding tissues.

In some embodiments of the invention, the micromechanical frequency beam can be configured to focus on a specific treatment area.

During the procedure, it is useful to constantly monitor the presence of acoustic contact between the transducer and the patient's body. This is especially true for UMB burst signals, which are most effective at low and micro intensities and are easily “lost” during procedures.

The signals for contact control optionally have a much lower intensity than the treatment signals.

In some embodiment of the device, the general structural diagram of which is shown in FIG. 21, the contact is determined by the microintensive circuit, in which the following is applied:
synchronous amplification by amplifier 32 of UWB spectra reflected from subcutaneous skin structures (converted into electrical by transducer 28);
processing by contact detector 34 of coded microsignals, which includes decoding and synchronous accumulation;
microprocessor processing and isolation from noise;
determination by contact detector 34 of the presence and quality of the contact;
transfer of information to executive microcontroller 36 and further to central processing unit (CPU) 16.

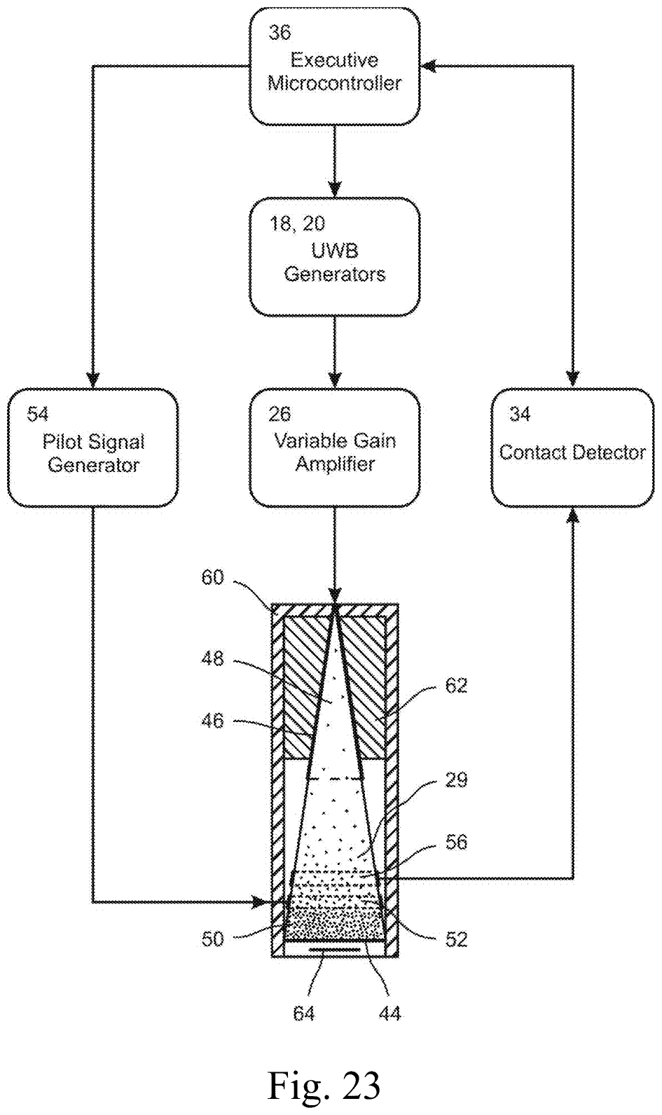

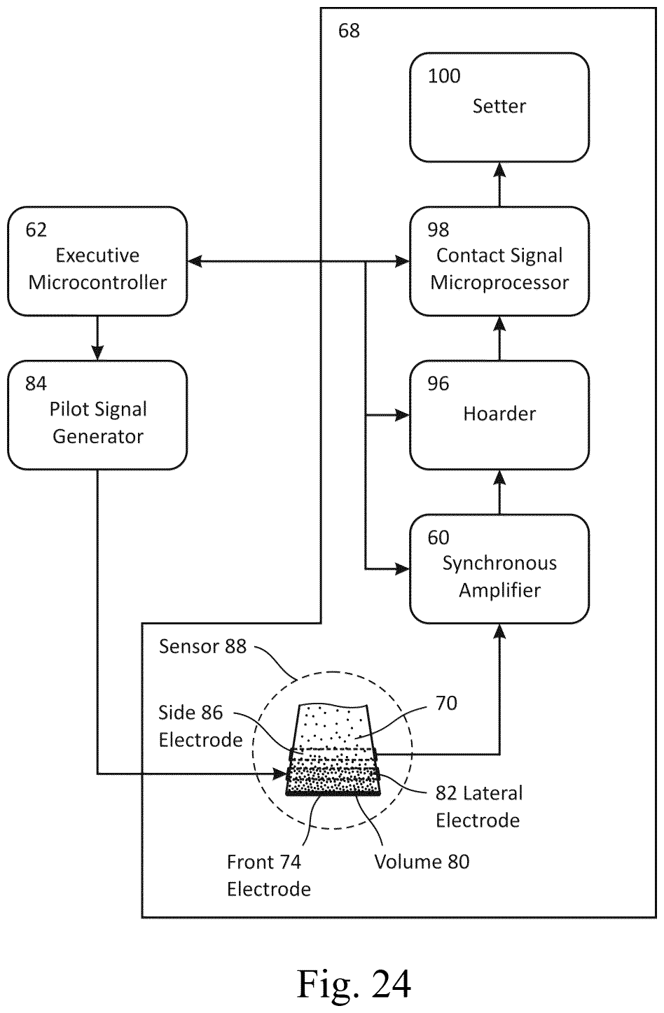

On FIG. 22 shows a variant structural diagram of mobile device 11 for UMB frequency burst regenerative cosmetology. Due to the limited power of the rechargeable power supply in this device, it is advisable to use volumetric multimode UWB transducer 29 (see also FIG. 23 and FIG. 24). The electromechanical conversion factor of such transducers, if they are made of piezoelectric ceramics, is 6 times higher than the best polymer PVDF transducers. In device when using polymer UWB transducers, it is useful to install additional variable gain amplifier unit 26.

In the main embodiment of personal UMB frequency burst device 11, the volumetric transducer is piezoceramic cone 29, on the front surface of which front electrode 44 is applied (see FIG. 3), and back electrode 46 is applied on the pointed back. The back part of transducer's volume 48 is depolarized. In the part of the volume of transducer 50, adjacent to front electrode 44, the polarization is preserved. Near electrode 44 on the side surface of the transducer, annular lateral electrode 52 is placed, which is used for supplying voltage from pilot signal generator 54 and excitation of one of the low-frequency oscillation modes of transducer 29, for example, the edge mode. The amplitude and decrement of the oscillation damping of the selected mode are sensitive to changes in the mechanical load when the acoustic contact changes. At a slightly greater distance from front electrode 44, second ring side electrode 56 is placed, the electrical signals from which are fed to synchronous amplifier 32 of contact detector 34. Lateral electrodes 52 and side electrode 56 perform the functions of T/R separator Volume 50, limited by the above electrodes, is a sensor 58 of contact (see FIG. 24).

Micromechanical transducer 29 is fixed with back part 48 in case 60 (see. FIG. 3) by means of damper 62. The front surface of transducer 29 protected by UWB protector 64, which is transparent to UMB frequency bursts in the ultra-wide frequency band 1-250 MHz.

In the technical implementation of device 11 (see FIG. 22) with volumetric micromechanical transducer 29, included in contact detector 34 functional blocks 32, 38, 40 and setter 68 (contact calibration signal) perform the same functions as in the device 10 in FIG. 21, but are simpler in execution.

For the contact control circuit with volumetric UMB burst transducer 29, contact pilot signal generator 44 was additionally used. In the Device 11 (see FIG. 22), in electronics unit 14, wireless data exchange unit 42 is additionally installed, which receives data from contact detector 34 and from which the data is transmitted to executive microcontroller 36. Remote central processor unit 16 communicates with unit 42 and controls the UMB frequency burst therapeutic device via Wi-Fi or Bluetooth.

In accordance with the main embodiment of the present device, these UWB generators 18 and 20 form continuous UWB spectra and, together with bandpass filter unit 22 and UWB spectrum corrector 24 (see FIG. 2) and UMB transducer 28, create therapeutic micromechanical frequency bursts having continuous UWB frequency bands inside the range of 1-250 MHz.

The required therapeutic total width of the UWB spectrum and the corresponding disease-dependent shapes of spectra indicated in the treatment protocol and recorded in the memory of central processing unit 16.

Each therapeutic UWB frequency band formed from one or more UWB bands, including ranges 1-3, 3-10, 10-30, 30-100, 100-250 MHz.

According to the basic embodiment of the present device 10 (see FIG. 21), the shape of spectra of at least one UWB main generator 18 is selected from a variety of main signals, including for example shock-wave, stress-wave, and from rectangular, triangular, trapezoidal signals spectra, or their differentials, or their combinations. Wherein shapes of spectra at least one correcting generator 20 is selected from a variety of signals, for example, from the differentials of the main pulse spectra, as well as low-cycle sinusoidal signal including, but not limited to, a Gaussian monocycle, a low-cycle sinusoid, a Sinus Cardinalis (Sinc-signal), or combinations thereof.

According to the basic embodiment of the present device 10, UMB transducer 28 is designed to introduce therapeutic ultra-wideband micromechanical frequency bursts into the treatment area through the conjunctival surface of the eye. UMB transducer 28 may be also designed to introduce ultra-wideband micromechanical frequency bursts into body entity openings, such as nasal cavity, oral cavity, esophagus, rectum, vagina.

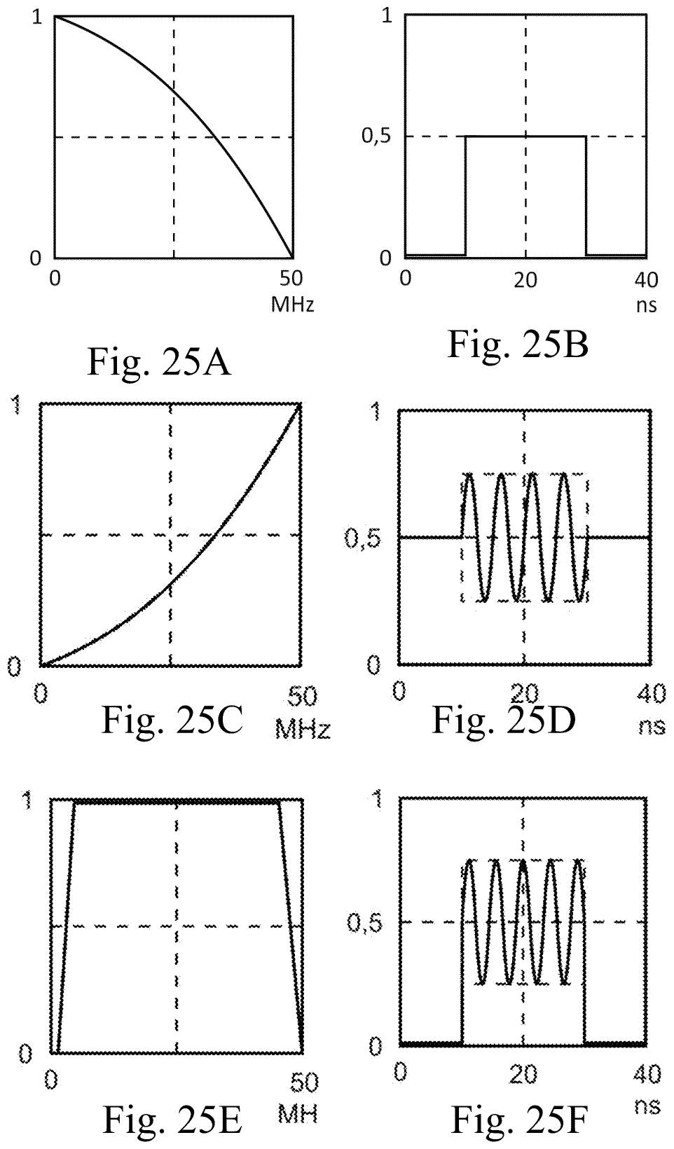

One of the preferred option of spectra and their corresponding time-domain waveforms of the UWB generators 18, 20 of the devices for UWB micromechanical regenerative frequency burst-therapy shown in FIGS. 25A to 25F:

FIG. 25A shows the main lobe of the shape of spectra of a rectangular video pulse.

FIG. 25B shows time-domain waveform a rectangular video pulse with a duration of 20 nanoseconds.

The spectrum in FIG. 25A may be unsuitable for a medical device due to an unacceptably large change in the frequency amplitudes in the operating frequency band of 1-50 MHz.

FIG. 25C shows the increasing part of the spectrum of a four-cycle sinusoid with a frequency of 55 MHz is given. The descending part of the spectrum with frequencies above 50 MHz is limited by the bandpass filter and is not shown in the figure.

FIG. 25D shows the time-domain waveform image of a four-cycle sinusoid.

FIG. 25E shows the result of summing the signals a) and c) by the spectrum corrector. With the correct choice of the number of cycles of the corrective signal and, accordingly, the steepness of the increase in the amplitude of its power frequency density, it may be possible to obtain a fairly flat total frequency characteristic of UWB bursts. By adding several corrective signals to the signal of the main generator, it may be possible to form any given, for example, increasing or arbitrary frequency characteristic in each UWB range, depending on the treatment protocol.

FIG. 25F shows the summed time-domain waveform signals.

The described device is applied as follows, in an exemplary embodiment of the invention.

By means of generators 18, 20, at least one pair of complementary electrical UWB spectra is generated, consisting of the main and corrective electrical signals. This pair of signals is intended for at least one ultra-wideband frequency range of treatment frequency burst

Then, the frequency bands of the main and corrective spectra are limited using bandpass filters unit 22 and the UWB spectra of the main and corrective electrical signals are corrected in at least one frequency range using the UWB corrector 24 of the signal spectrum shapes by summing them to obtain UWB bursts with a given spectrum shape.

The obtained UWB electrical spectra are converted into therapeutic UWB micromechanical frequency bursts. These bursts are delivered through an acoustically transparent UWB transducer 28 and a UWB conductive medium to the surface of the body. Then, non-invasive introduction and delivery of UWB micromechanical frequency bursts to the treatment area and surrounding tissues adjacent to the mentioned area is carried out. In this case, there may be an energy transfer of therapeutic UWB micromechanical frequency bursts to a variety of mechanosensitive tissue structures, including cellular ensembles, intercellular matrix, cells and cell nuclei. This process may be accompanied by UWB micromechanical frequency burst-stimulation of regenerative processes at various levels of resonant mechanosensitive tissue structures, including reprogramming of some somatic cells into multipotent and pluripotent stem cells and, consequently, acceleration and enhancement of natural regenerative processes. UWB micromechanical frequency burst-stimulation of the processes of accumulation of stem multipotent and pluripotent cells inside and around the treatment area may be carried out by repeating therapeutic micromechanical bursts for many days.

The first therapeutic UWB micromechanical frequency burst, formed by the first main and complementary to it first corrective spectra, has the first frequency band, which may have a stressful effect on the tissues and the extracellular matrix of the treatment area. However, at least one more, the second therapeutic UWB micromechanical frequency burst, formed by the second main and complementary second corrective spectrum, has at least a second frequency band. The proposed method, in turn, may stimulate in the somatic cells of the treatment area processes of reprogramming, direct reprogramming, differentiation, proliferation, tissue formation and replacement of diseased or damaged cells with healthy cells.

The first treating UWB micro-mechanical frequency bursts may be formed at least in the first frequency ranges 1-3, 3-10 MHz and have a frequency, space and time averaged intensity of bursts in the region treatment of 1-30 mW/cm².

In the same time, the second treating UWB micro-mechanical frequency bursts may be formed in frequency ranges of 10-30, 30-100, 100-250 MHz, with a frequency, space and time averaged intensity of frequency bursts on the body surface of 0.001-100 mW/cm² and in the treatment area of 0.0001-30 mW/cm².

Herewith the treating UWB micromechanical frequency bursts in the frequency ranges 1-3, 3-10, 10-30 MHz may repeat during the treatment with a frequency of 0.05-1 KHz, and in the frequency ranges 30-100, 100-250 MHz may be repeated at a frequency of 0.05-103 KHz.

According to this invention, the UWB micro-mechanical frequency bursts, formed by proposed device may be used in regenerative medicine to treat diseases selected from the group, including: stroke, myocardial infarction, ischemia, spinal cord injury, degradation of the retina and optic nerve, glaucoma, diseases of the musculoskeletal system, diabetes, some types of cancer, atherosclerosis, varicose veins, post-traumatic, post-burn and postoperative wounds, rejuvenation of the skin and other structures of tissues and organs of the body.

Moreover, the treating ultra-wideband micro-mechanical frequency bursts, formed by the device may be used in regenerative cosmetology to treat diseases and skin conditions including signs of skin aging, scars, acne, wrinkles, melasma, swelling, edema.

According to the proposed invention, shapes of spectra of the generators are selected from a variety of signals (see FIG. 25), including for example shock-wave, stress-wave, and also from: rectangular, triangular, trapezoidal pulses, combinations, and the shapes of the spectra of corrective generators are selected for example from a variety of main spectra and their differentials, as well as from a Gauss monocycle, a low-cycle sinusoid, a cardinal sinusoid (Sinc signal), non-periodic arbitrary waveforms, or combinations thereof.

In addition to the previous one, the sequence of therapeutic frequency bursts can be coherent, as well as incoherent.

The transducing of electrical spectra into micro-mechanical treating frequency bursts can be performed by more than one UWB transducers, acoustically coupled to the ultra-wideband acoustically transparent protector.

The treating UWB micro-mechanical frequency bursts from transducer 29 (see FIGS. 23. 24) are applied to the body surface through acoustically transparent and acoustically coupled to each other elements, such as thin ultra-wideband protector 64, a thin contact layer or an extended medium, for example, water, placed between the protector and the body surface.

The ultra-wideband electrical spectra arriving at transducer 29 (see FIGS. 23 and 24) excite the oscillations of thin piezoelectric layer of this transducer 29 adjacent to front electrode 44 on its radiating front surface. Micromechanical planar P-mode frequency bursts are input a protective ultra-wideband protector 64. Some shape of spectra of treatment frequency bursts are shown in FIG. 25. The micromechanical frequency bursts introduced into/through the patient's skin through an ultrasound gel such as UltraGel manufactured by many companies.

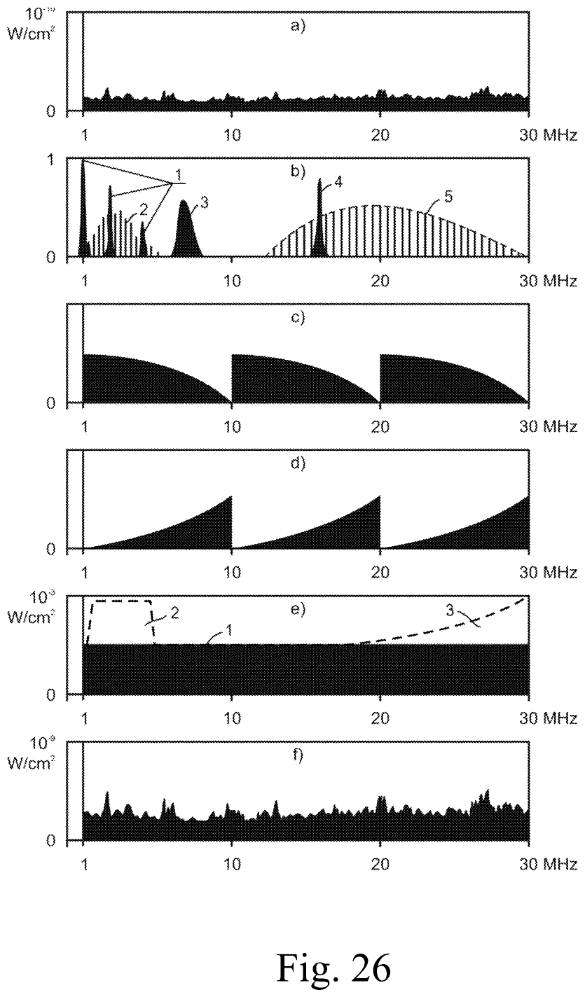

FIG. 26 shows a comparison of the hypothetical spectra of natural oscillations of bioconstructions of biological tissue with the energy spectra of known pulses of ultrasonic medical devices and with the energy spectra of the proposed ultra-wideband micromechanical frequency devices.

Schemes a), b), c), d), e), f) in FIG. 26 depict:
a) two-dimensional spectrum of natural micromechanical oscillations of the biostructure (our hypothesis);
b) spectra of ultrasonic vibrations of known therapeutic devices, where:
1—Classic devices. Ionto H&B and Duarte R. LIPUS device [24];
2—Tyler W. Devices with pulse technology LIPUS [30];
3—Marchenko A. Broadband tunable therapeutic apparatus. [67];
4—Kruglikov I. High-frequency ultrasound apparatus for therapy and cosmetology [80];
5—Tyler W. Ultrasonic pulse high-frequency apparatus [57].
b) spectra of ultrasonic vibrations of known therapeutic devices, where: 1—Classic devices. Ionto H&B and Duarte R. LIPUS device [24]; 2—Tyler W. Devices with pulse technology LIPUS [30]; 3—Marchenko A. Broadband tunable therapeutic apparatus. [67]; 4—Kruglikov I. High-frequency ultrasound apparatus for therapy and cosmetology [80]; 5—Tyler W. Ultrasonic pulse high-frequency apparatus [57].
c) according to the invention: the spectra of three main signal generators with center frequencies of 1, 10 and 20 MHz after bandpass filters;
d) according to the invention: the spectra of three correcting signal generators with center frequencies of 10, 20 and 30 MHz after bandpass filters;
1—idealized energy spectrum, obtained by summing the main and correcting signals and after converting them into micromechanical frequency bursts;
2.—ultra-wideband spectrum in the frequency band of 1-5 MHz to enhance apoptosis processes;
3.—energy spectrum with increasing power frequency density to compensate for the frequency-dependent absorption of micromechanical energy in biostructures, and
e) according to the invention: 1—idealized energy spectrum, obtained by summing the main and correcting signals and after converting them into micromechanical frequency bursts; 2.—ultra-wideband spectrum in the frequency band of 1-5 MHz to enhance apoptosis processes; 3.—energy spectrum with increasing power frequency density to compensate for the frequency-dependent absorption of micromechanical energy in biostructures, and
f) natural two-dimensional oscillations of biostructure enhanced by micromechanical ultra-wideband frequency bursts.

For comparison, (see FIG. 26) we used the well-known LIPUS pulsed ultrasonic signals, namely their most studied version with a filling frequency of long pulses of 1.5 MHz. This LIPUS and all its known variants are narrowband signals.

Broadband ultrasound signals are known in diagnostics, but their intensity is thousands of times greater than UMI Burst and LIPUS. We have not found publications about the therapeutic application of broadband or ultra-wideband ultrasound in the literature available to us, with the exception of patents and articles by Marchenko A. [88, 90,91,93,94] and Tereshchenko N. [92]. But in these sources also did not use and do not describe pulsed broadband therapeutic signals, because these signals were either harmonic, tunable in frequency, i.e., monofrequency, or stochastic, arbitrarily tunable in frequency, i.e., also narrowband.

W. Tylor in his patents [49, 50, 68, 71, 72, 77] suggests using known standard pulses, arbitrary waveform pulses, or combinations thereof for treatment. At the same time, the text of these patents always refers to the treatment with ultrasound of a certain frequency or group of frequencies. Therefore, we perceive the signals proposed by W. Tylor as a sequence of repetitive pulses, in which, due to coherence, the frequencies necessary for treatment or their sets in the form of line spectra or comb spectra are formed. Such spectra are not broadband, but narrowband.

Even if W. Tylor had specially formed broadband pulses with continuous spectra, for example, by their incoherent generation, the energy of the spectra of such pulses would have been concentrated mainly in the low-frequency part of the spectrum.

To transfer energy in a wide range, it may be useful not only to use at least two pulses with mutually complementary spectra, but also to filter them, correct the shape of the power density spectrum of each pulse, and sum up many pulses, which was done by us.

A single pulse of any shape has a power frequency density decreasing with frequency, sometimes, as for a cardinal sinusoid, falling very sharply. The pulse energy is always concentrated in the low-frequency part of the energy spectrum. For the purposes of regenerative therapy, it may be useful to transfer energy to biostructures in the across ultra-wideband frequency spectrum, within the range of 1-250 MHz.

Therefore, the energy spectrum of one basic, main known impulse may usefully be supplemented with the spectra of other impulses, which may be located on different parts of the frequency axis. The spectra of impulses may be united in amplitude and frequency, which means that before combining the pulse spectra, it may be useful to limit their frequency bands (that is, apply bandpass filtering) and select the amplitude characteristics of the frequency power densities of the spectra that complement the main spectrum to the desired one.

Thus, we will form one or more combined ultra-wideband energy spectra with a distribution of frequency power densities that we have specified, which are configured to transfer ultra-wideband frequency energy to mechanosensitive elements of the biostructures. In this way, it is possible to form ultra-wideband continuous spectra with constant or increasing or arbitrary amplitudes of frequency power densities along the frequency axis.

We use the spectra of known impulses as components (letters) of new impact frequency signals. From the spectra (letters) of standard or modified by us impulses, we form new ultra-wideband spectra (words) with a given distribution of frequency power densities.

This is how we create a modulating of cellular activity (healing) ultra-wideband frequency energy image—an energy bursts or splashes which transfers energy to a huge number of mechanosensitive elements of biostructures, supplementing the energy of their own thermal vibrations and improving the interaction between elements of biostructures.

The therapeutic ultra-wideband micromechanical frequency bursts, obtained by us and described above, are further used as follows.

The treating ultra-wideband micro-mechanical frequency bursts are introduced into the treatment area through the conjunctival surface of the eye, as well as directly into the orifices of the body, such as the nasal cavity, oral cavity, esophagus, rectum, vagina.

The impact on the treatment area with ultra-wideband micromechanical frequency bursts is carried out daily for 5-20 minutes for 5-60 days.

The authors of proposed device explain the therapeutic effect of its as follows. Biostructures of body tissues oscillate on a set of interconnected frequencies, and each level of cellular organization corresponds to its own UWB frequency band. According to the tensegrity concept, bio tissues, cell ensembles, intercellular matrix, cells, cytoskeletons, microtubule system, filaments, nuclei and DNA are band oscillatory systems. Impact on the body ultra-wideband micromechanical frequency burst allows it to receive additional energy, which improves and enhances information exchange at various levels of cellular organization, increases the number (volume) of healthy cells involved in the process of influencing the sick and, thus, up to 3 times enhances and accelerates during treatment many of the natural indicators of regeneration and repair.

This may happen through the following processes:
ultra-wideband micromechanical reprogramming of some somatic cells into stem multipotent and pluripotent ones;
UWB micromechanical frequency burst-stimulation of the processes of accumulation of stem multipotent and pluripotent cells in/around the treatment area;
enhancing the proliferation of stem cells existing in the treatment area and surrounding tissues and replacing old and diseased cells with new healthy ones in the tissues;
an increase in the concentration of stem cells in the treatment area and surrounding tissues due to the acceleration of epigenetic of reprogramming of somatic cells;
stimulation of the processes of formation and functioning of biomolecular condensates inside the cells. These membraneless bodies perform many biological functions, including cell fate determination, signal transduction, regulation of gene expression, and protein translation [79].
an increase in the number of stem cells in the treatment area and surrounding tissues due to the UMB frequency burst of stimulation of the homing effect;
increased activity in the treatment area and surrounding tissues of innate immunity cells (macrophages);
an increase in the immunomodulatory potential of tissues due to the production of many active products (cytokines, chemokines, growth factors) by stimulated macrophages, which affect the cellular environment and stimulate the processes of repair of damaged tissues.
suppression of foci of chronic inflammation in the area of pathology, and, consequently, stopping the pathological processes of tissue degeneration;
an increase in the activity of innate immunity, namely, the acceleration of blast transformation of lymphocytes.

In general, there may be an acceleration and intensification of reparative-regenerative processes at various levels of mechano sensitive tissue structures.

The proposed invention can be applied for the treatment of traumatic and degenerative pathological changes in organs and tissues, cardiovascular, endocrine and skin diseases, as well as pathologies of the musculoskeletal system and other diseases. The application of the invention is promising for reducing hypoxic lesions of the heart and brain, restoring the immune system by stimulating the protective forces and general endurance, as well as for preventing premature aging.

The invention can also be applied in military and sports medicine, aesthetic medicine and cosmetology.

It is obvious that the present invention is not limited to the above-mentioned embodiments, and variations and modifications may be made without departing from the scope of the present invention. It will be appreciated by persons skilled in the art that the present invention is not limited by the drawings and description hereinabove presented.

### EXAMPLES

Some examples are described:

1. A device for ultra-wideband (UWB) micromechanical regenerative frequency burst therapy, containing at least one main generator of UWB main electrical spectra, at least one correcting generator of UWB correcting electrical spectra, delayed along the frequency axis relative to the main spectra, and complementary to the main spectra, the band-pass filters unit, that serves to select the frequency bands of the main and corrective spectra, UWB spectrum shape corrector, UWB variable gain amplifier, ultra-wideband transducer of the electric spectrum into the micromechanical frequency bursts (UMB transducer), UWB protector and executive microcontroller, central processor unit and microprocessor for monitoring the presence of contact of the transducer with the patient's body, in which:
a) at least one main generator of UWB main electrical spectra connected to the first input of the ultra-wideband spectrum shape corrector through the bandpass filter's unit;
b) at least one correcting generator of UWB correcting electrical spectra connected through the bandpass filters unit to the second input of said ultra-wideband spectrum shape corrector, wherein the output of said corrector is connected to the input of the UWB variable gain amplifier, and the output of the latter is connected to the electrodes of the UMB transducer, which is acoustically coupled to the UWB protector, in turn acoustically connected through the contact medium with the surface of the patient's body;
c) all of the above elements of the device are designed to form the electrical spectra necessary for the treatment of UWB, their subsequent conversion into UWB micromechanical frequency bursts, the shapes of the spectra of which are different from those directed downwards, as well as to introduce these frequency bursts through the surface of the patient's body into the treatment area and neighboring healthy tissues in order to influence all cellular structures with micromechanical UWB frequency bursts and cause their multiple own oscillations, thereby forming micromechanical therapeutic “images” identical to natural images that occur during the life of cells and tissues;
d) in addition, the specified device for ultra-wideband micromechanical frequency burst regenerative therapy also contains an executive microcontroller connected to the inputs of the main and corrective generators, and this microcontroller is also a programmer and a status indicator of this device, which in turn is controlled by the central processing unit (CPU).

2. The device according to example 1, in which the mentioned UWB spectrum generators together with band-pass filter unit and the UWB spectrum shape corrector and the UMB transducer form therapeutic UWB micromechanical frequency bursts with spectra within the frequency band of 1-250 MHz, which has spectrum shapes different from downward, while the mentioned band consist of one or more ultra-wideband frequency ranges, including 1-3, 3-10, 10-30, 30-100, 100-250 MHz.

3. The device according to example 2, which contains more than two generators of UWB spectra, which, together with band-pass filter unit the UWB spectrum shape corrector and the ultra-wideband micromechanical frequency burst transducer create UWB micromechanical frequency bursts, forming a continuous frequency band, and this UWB band (spectrum) may consist of several UWB ranges.

4. The device according to example 1, in which the said executive microcontroller also performs the functions of an indicator of the current state of this device and is connected to a detector of the presence and degree of acoustic contact between the said UMB transducer and the patient's skin, which in its queue contains:
a sensor for determining the parameters of the contact, formed by the frontal and lateral electrodes of the micromechanical volumetric UMB transducer and the sensing part of the UMB transducer volume located between the electrodes,
synchronous amplifier,
hoarder of control signals,
setter of values of parameters of signals of presence and degree of contact,
signal processor of the contact detector.

5. The device according to example 4, wherein said executive microcontroller is connected to at least two UWB spectrum generators of main and correcting spectra, and to a narrowband low frequency LF pilot signal generator, to synchronous amplifier, to hoarder and contact signal microprocessor.

6. The device according to example 1, which contains a side electrode placed on the back surface of the UMB transducer, which is connected in series with the first input of the synchronous amplifier, the said hoarder, the contact signal microprocessor, and with the second input of the executive microcontroller, and the third output of the mentioned microcontroller is connected to the second input of the synchronous amplifier, and the signal from the third output of the microcontroller is delayed relative to the signal of the first output by more than on 0.2 μs.

7. The device according to examples 1-6, which consists of a master part—a remote central processing unit and an executive part—a mobile applicator for non-invasive regenerative UWB micromechanical frequency burst therapy with the electronics unit placed inside or outside the mobile applicator, moreover, the master part is CPU—central processing unit, connected to the executive microprocessor via Bluetooth, Wi-Fi or a radio module, and is an autonomous unit, performed as a mobile phone, tablet, mobile or desktop computer, and the executive part also contains a Bluetooth or Wi-Fi or a radio module and a rechargeable power source—with a charging microcontroller.

8. The device according to example 1, in which the amplitude distribution of the frequency power density of spectrum of at least one main generator is selected from a variety of main signals, including shock-wave, stress-wave, and from rectangular, triangular, trapezoidal form of bursts or their differentials, or their combinations, and the various amplitude distribution of frequency power density of at least one correcting signal is selected from a variety of signals, namely, from the differentials of the main pulse signals, as well as low-cycle sinusoidal signal including, a Gaussian monocycle, a low-cycle sinusoid, a Sinus Cardinalis (Sinc signal), or combinations thereof.

9. The device according to example 7, in which the applicator is designed to introduce treating ultra-wideband micromechanical frequency bursts into the treatment area through the conjunctival surface of the eye.

10. The device according to example 7, wherein the applicator is designed to introduce ultra-wideband micromechanical frequency bursts into body entity openings, such as nasal cavity, oral cavity, esophagus, rectum, vagina.

11. A method of operation of the device for UWB regenerative micromechanical frequency burst-therapy, comprising:
generating at least one pair of complementary electrical UWB signals consisting of the main and corrective electrical signal with different amplitude distributions of the power frequency density, moreover the corrective spectrum is delayed along the frequency axis relative to the spectrum of the main spectra, both are designed for one ultra-wideband frequency range of treating signals within the band 1-250 MHz;
correction of the UWB spectra of the main and corrective electrical signals in at least one frequency range using band-pass filters and spectrum corrector, moreover, the corrective spectra are delayed along the frequency axis relative to the main ones, and the total spectra differ from the descending ones.
conversion by UMB transducer of UWB electrical spectra into treatment UWB micromechanical frequency bursts;
delivery of treating UWB micromechanical frequency bursts through acoustically transparent UWB protector and UWB conductive medium to the body surface;
cause all possible own micromechanical oscillations of cells and tissues, thus forming UWB micromechanical therapeutic “images” identical to natural images that arise in the process of life, that is, cause the radiation of own micromechanical therapeutic signals in the entire ultra-wide frequency range of 1-250 MHz;
non-invasive input and delivery of micromechanical UWB frequency bursts to the affected area and tissues adjacent to it, and the burst field is configured so that: cause all possible own micromechanical oscillations of cells and tissues, thus forming UWB micromechanical therapeutic “images” identical to natural images that arise in the process of life, that is, cause the radiation of own micromechanical therapeutic signals in the entire ultra-wide frequency range of 1-250 MHz;
in the 1-5 MHz range—stimulation of regenerative processes in biological tissues, acceleration of growth of tissues, nerves and blood vessels, wound healing;
in the 1-10 MHz range—excitation of the activity of the extracellular matrix, increased apoptosis, increased local immunity, increased proliferative and migration processes and the activity of stem cells existing in tissues;
in the 10-30 MHz range—stimulation of protein synthesis, stimulation of stem cell programming, direct reprogramming of progenitor cells, stem cell reproduction, a general increase in local cellular activity and repair processes; in the 30-100 MHz range—an increase in the activity of processes caused by deformations of the nuclear membranes, processes of epigenetic reprogramming, gene expression, transformation of somatic cells into stem cells;
or to induce your own frequency micromechanical treatment responses in only one ultra-wide range or several ultra-wide ranges, for example: in the 1-5 MHz range—stimulation of regenerative processes in biological tissues, acceleration of growth of tissues, nerves and blood vessels, wound healing; in the 1-10 MHz range—excitation of the activity of the extracellular matrix, increased apoptosis, increased local immunity, increased proliferative and migration processes and the activity of stem cells existing in tissues; in the 10-30 MHz range—stimulation of protein synthesis, stimulation of stem cell programming, direct reprogramming of progenitor cells, stem cell reproduction, a general increase in local cellular activity and repair processes; in the 30-100 MHz range—an increase in the activity of processes caused by deformations of the nuclear membranes, processes of epigenetic reprogramming, gene expression, transformation of somatic cells into stem cells;
transfer of treating energy of UWB micromechanical frequency bursts to a variety of mechano-sensitive biological structures, including cellular ensembles, extracellular and intercellular matrices, blood vessels, blood cells, nervous system, immune and stem cells, cytokines, chemokines and other biologically active factors, cell nuclei and DNA;
generally, UWB micromechanical frequency burst-stimulation or modulation of regenerative processes at various levels of the aforementioned mechanosensitive tissue structure, including reprogramming of some somatic cells into multipotent, progenitor and pluripotent stem cells and, consequently, acceleration and enhancement of natural regenerative processes;
UWB micromechanical frequency burst stimulation of the processes of accumulation of pluripotent, multipotent, progenitor stem cells and immune cells in and around the treatment area by repeating therapeutic micromechanical frequency bursts act for many, up to 60 days.

12. The method according to example 11, according to which at least one the first therapeutic UWB micromechanical frequency burst, formed by the first main and complementary to it first correcting spectrum, has the first frequency range with the first form of the spectrum, which has a stressful effect on the tissues and on apoptosis processes in by changing the state of the extracellular matrix in the affected area and surrounding healthy tissues, and at least one the second therapeutic UWB micromechanical frequency burst, formed by the second main and additional second correcting spectra, has at least a second frequency range with the second form of the spectrum, and stimulates reprogramming and direct reprogramming processes in the affected area and surrounding healthy tissues, and differentiation, proliferation and replacement of diseased or damaged cells by healthy ones.

13. The method according to example 11, in which the first therapeutic UWB micromechanical frequency bursts formed in the first frequency range of 1-3, 3-10 MHz, and have in the treatment area an intensity averaged over frequency, space and time in the range of about 1-500 mW/cm².

14. The method according to example 11, in which the second treating UWB micro-mechanical frequency bursts are formed in frequency ranges of 10-30, 30-100, 100-250 MHz, and have an intensity of frequency bursts averaged over the frequency of time and space on the body surface of 0.001-100 mW/cm² and in the treatment area of 0.0001-30 mW/cm².

15. The method according to example 11, in which a plurality of these first and second therapeutic UWB micromechanical frequency bursts form a common band of the therapeutic spectrum and are repeated during treatment in the frequency ranges 1-3, 3-10-30 MHz with a frequency of 0.05-1 kHz, and in the frequency ranges of 30-100, 100-250 MHz with a frequency of 0.05-10³ kHz.

16. The method according to example 11, whereby the UWB micro-mechanical frequency bursts, formed by said device are used in regenerative medicine to treat diseases selected from the group, including: stroke, myocardial infarction, ischemia, spinal cord injury, degradation of the retina and optic nerve, glaucoma, diseases of the musculoskeletal system, diabetes, some types of cancer, atherosclerosis, varicose veins, post-traumatic, post-burn and postoperative wounds, rejuvenation of the skin and other structures of tissues and organs of the body.

17. The method according to example 11, whereby the treating ultra-wideband micro-mechanical frequency bursts, formed by the device are used in regenerative cosmetology to treat diseases and skin conditions including signs of skin aging, scars, acne, wrinkles, melasma, swelling, edema,

18. The method according to example 11, in which the shapes of burst spectra of the main generators are selected from a variety of spectra, including shock wave, stress wave, as well as from the spectra of rectangular, triangular, trapezoidal signals or combinations thereof, and the shapes of the spectra of corrective generators are selected from a variety of main signal spectra and their differentials, as well as from a Gauss monocycle, a low-cycle sinusoid, a cardinal sinusoid (Sinc signal), arbitrary waveforms, or combinations thereof.

19. The method according to example 11, according to which the sequence of treating frequency bursts can be coherent, as well as incoherent.

20. The method according to example 11, whereby the transducing of electrical spectra into micromechanical treating frequency bursts is performed by more than one UMB transducers.

21. The method according to example 11, whereby the conversion of electrical impulses into micromechanical therapeutic frequency bursts is carried out by several ultra-wideband transducers, which are located in a single housing, with the ability to move inside the latter in three planes.

22. The method according to example 11, whereby the treating UWB micro-mechanical frequency bursts are applied to the body surface through acoustically transparent and acoustically coupled to each other elements, such as an ultra-wideband protector, a contact layer or an extended contact medium, placed between the protector and the body surface.

23. The method according to example 11, whereby treating ultra-wideband micro-mechanical frequency bursts are introduced into the treatment area through the conjunctival surface of the eye, as well as directly into the orifices of the body, such as the nasal cavity, oral cavity, esophagus, rectum, vagina.

24. The method according to example 11, whereby the therapeutic impact on the pathological area and surrounding healthy tissues with ultra-wideband micromechanical frequency bursts is carried out daily for 5-40 minutes for 5-60 days.

The invention relates to devices and methods of their operation for the restoration of diseased or damaged tissues of the human body by extracorporeal acceleration of natural reparative processes induced by micromechanical frequency burst at various levels of organization of tissue and cellular life, including the transformation of somatic cells into multipotent and pluripotent stem cells, accumulation of these cells and accelerated safe replacement of old cells with new cells. The proposed device for ultra-wideband (UWB) micromechanical regenerative frequency burst-therapy contains at least one main generator of the ultra-wideband (UWB) electrical signals, at least one generator of the corrective UWB electrical signals, band-pass filter unit, an UWB corrector of signals frequency spectra and amplitudes, an UWB amplifier, an UWB micromechanical transducer, an UWB protector and main controller. All these elements of the device are designed to generate UWB electrical impulse signals, convert them into UWB micromechanical frequency bursts signals with a constant or frequency-increasing amplitude in at least one UWB frequency range and input these bursts through the surface of the patient's body into the impact zone and adjacent healthy tissue. The main controller is connected to the outputs of the main and corrective generators, and it is also a programmer and an indicator of the current state of the entire device. A method and operating parameters of this device for UWB micromechanical regenerative frequency burst therapy are also proposed.

It is expected that during the life of a patent maturing from this application many relevant ultrasound generators will be developed and the scope of the term ultrasound generator, or ultrasound generating element, is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A therapeutic device for applying impact ultra-wideband ultrasound bursts to a target location inside a biological object; said device comprising:
a generating module comprising at least one ultrasound generator configured for generating at least one ultra-wideband electrical signal of ultrasound frequency;
at least one ultra-wideband ultrasound transducer connected to said at least one ultrasound generator and attachable to said biological object in proximity to an object area to be treated; said at least one ultra-wideband ultrasound transducer configured for converting said at least one electric ultra-wideband frequency signal into ultra-wideband ultrasound impact burst and transmitting thereof into said biological object target location;
a control unit configured for controlling said at least one ultrasound generator such that said at least one electric ultra-wideband ultrasound electrical signal of ultrasound frequency is generated;
wherein said at least one transmitted ultra-wideband ultrasound impact burst comprises a low-frequency range having a duration ranging between 0.5 and 1 μs at a frequency ranging between 1 MHz and 3 MHz and a high-frequency range having a duration ranging between 10 and 50 ns at a frequency higher than 3 MHz within a range of a frequency factor greater than 3; transmitting said high-frequency and low-frequency ranges is synchronized such that a period of transmitting said high-frequency range is within a period of transmitting said low-frequency range to said biological object target location.

2. The therapeutic device according to claim 1, wherein said at least one ultra-wideband ultrasound impact burst in said high-frequency range is transmitted in a middle of said duration of transmitting said at least one ultra-wideband ultrasound burst in said low-frequency range to said biological object target location.

3. The therapeutic device according to claim 1, wherein said at least one ultrasound generator is configured for generating a set of said ultra-wideband ultrasound impact bursts.

4. The therapeutic device according to claim 1, wherein said generating module comprises at least one ultrasound generator configured for consecutively generating a plurality of predetermined signals and a buffer configured for accumulating consecutively generated signals and in-phase transmitting said predetermined signals generated by said at least one generator and accumulated in said buffer to said at least one ultrasound transducer.

5. The therapeutic device according to claim 1, wherein said generating module comprises at least two ultrasound generators configured for generating said at least two ultra-wideband ultrasound signals at frequencies within said low-frequency and high-frequency ranges and supplementing each other and a corrector configured for band-pass filtering said at least two ultra-wideband ultrasound signals in said low-frequency and high-frequency ranges, amplitude normalizing said at least two ultra-wideband ultrasound signals and in-phase summing said at least two ultra-wideband ultrasound electric signals such that a summed ultra-wideband ultrasound signal when transmitted to said at least one ultrasound transducer causes generating said at least one ultra-wideband ultrasound impact burst.

6. The therapeutic device according to claim 1 comprising a laser module configured for illuminating biological object area to be treated; said laser module comprises a laser infrared light source configured for generating a laser beam and a light modulator configured for modulating intensity of said laser beam at to generate ultrasound of greater than 20 MHz.

7. The therapeutic device according to claim 6, wherein said laser module comprises an optical element configured for focusing a modulated laser beam on a target location-of said object.

8. A method of applying ultra-wideband ultrasound impact bursts to a biological object area; said method comprising steps:
providing a therapeutic device of claim 1;
connecting at least one ultrasound transducer to said at least one ultrasound generator;
attaching said at least one ultrasound transducer to said biological object-in proximity to a biological object area to be treated;
generating at least one-dual-band electric ultra-wideband ultrasound electric signal;
converting said at least one electric ultra-wideband ultrasound electric signal by said at least one ultrasound transducer into at least one ultra-wideband ultrasound impact burst;
transmitting at least one ultra-wideband ultrasound impact burst into target location of said biological object;
wherein at least one transmitted ultra-wideband ultrasound impact burst comprises a low-frequency range having a duration ranging between 0.5 and 1 μs at a frequency ranging between 1 MHz and 3 MHz and a high-frequency range having a duration ranging between 10 and 50 ns at a frequency higher than 3 MHz within a frequency range of a frequency factor greater than 3;
herewith transmitting said high-frequency and low-frequency ranges are in-phase transmitted to said biological object such that a period of transmitting said high-frequency range is within a period of transmitting said low-frequency range.

9. The method according to claim 8, wherein said step of transmitting at least one ultra-wideband ultrasound impact burst in said high-frequency range is transmitted in a middle of said duration of transmitting said at least one ultra-wideband ultrasound burst in said low-frequency range to said biological object.

10. The method according to claim 8, wherein said step of generating at least one electric ultra-wideband ultrasound electric signal by said at least one ultrasound generator comprises generating a set of said ultra-wideband ultrasound impact bursts.

11. The method according to claim 8, wherein said step of generating at least one electric ultra-wideband ultrasound electric signal comprises consecutively generating a plurality of predetermined signals by said at least one ultrasound generator and accumulating signals consecutively generated by said at least one ultrasound generator in a buffer and synchronically transmitting said predetermined signals to said at least one ultrasound transducer.

12. The method according to claim 8, wherein said step of generating at least one electric ultra-wideband ultrasound electric signal comprises at least two ultra-wideband ultrasound signals at frequencies within said low-frequency and high-frequency ranges and supplementing each other; said at least two ultra-wideband ultrasound signals are band-pass filtered in said low-frequency and high-frequency ranges, amplitude normalized, and synchronously summed such that a summed ultra-wideband ultrasound signal when transmitted to said at least one ultrasound transducer, causes generating said at least one ultra-wideband ultrasound impact burst.

13. The method according to claim 8 comprising a step of illuminating said biological object area to be treated by an infrared laser module; said step of illuminating said biological object to be treated further comprising consecutively performed sub-steps of generating an infrared laser beam by a laser light source and modulating intensity of said laser beam for generating a laser beam and modulating intensity of said laser beam to generate ultrasound with greater than 20 MHz.

14. The method according to claim 13, wherein said step of illuminating said biological object area to be treated comprises focusing a modulated laser beam on a surface of said biological object by a focusing optical element.
